SEQUENCE LISTING (79 sequences)

SEQ ID NO: 1 - protein, 590 aa, Zea mays
MPLRQRPTMGGGGGGVAKMPAGQLWLLLLGVLLLAFGVPAQASRNTHYDFVITETKVTRLCHEKTILAVNGQFPGPTIYARKDDVVIVNVYNQGYKNITLHWHGVDQPRNPWSDGPEYITQCPIQPGANFTYKIIFTEEEGTLWWHAHSEFDRATVHGAIVIHPKRGTVYPYPKPHKEMPIILGEWWNADVEQILLESQRTGGDVNISDANTINGQPGDFAPCSKEDTFKMSVEHGKTYLLRVINAGLTNEMFFAVAGHRLTVVGTDGRYLRPFTVDYILISPGQTMNMLLEANCATDGSANSRYYMAARPFFTNTAVNVDDKNTTAILEYTDAPPSAGPPDSPDLPAMDDIAAATAYTAQLRSLVTKEHPIDVPMEVDEHMLVTISVNTIPCEPNKTCAGPGNNRLAASLNNVSFMNPTIDILDAYYDSISGVYEPDFPNKPPFFFNFTAPNPPQDLWFTKRGTKVKVVEYGTVLEVVFQDTAILGAESHPMHLHGFSFYVVGRGFGNFDKDKDPATYNLVDPPYQNTVSVPTGGWAAMRFRAANPGVWFMHCHFDRHTVWGMDTVFIVKNGKGPDAQMMPRPPNMPKC

SEQ ID NO: 2 - DNA, 3491 bp, Zea mays
accgactggtggcggcatgacgaacgaaacatgcatatgcattcgtcccctcgtcgtcgttggcagctctcgctcctctataaataccagcgccatccgcttcagatgagcatcgatcccagcaacgcacggagcgtacgtacattgcagtagctagctatagctggccggccatcccctctcgctcgctgctaaacacgttccagcttgtttgctcagagaaacagcgcgcgcgcacacacacacacatcatcatcatcgattcatcgtacacaggatcagagagcttaattagttctagctctgctgcatgccccttcgacaacgtccgacgatgggcggcggcggcggcggtgtagctaagatgccggcaggccagctctggttattactgctaggcgtgttgttgttagcatttggagtcccagcccaggcctccaggaatactcactacgacttcgttgtaagttgatcgatctcgacgatatatctcaatatctcatttcgactttatattaccggccaatttaatttcagagacgcaggcagttacctgagaacgttagtgctagcttgcgggcaccttacaaataatcagccgctgtacatgcgtgttctagttttcaaataaatgtctagctactaggttgatgtgattcatgtttgcatggcttcagaacatttgaattcaagtgtctagctaggtctaggtacgtagtctatctgattggttgcccgtatcgctttattctggtcctctgcgtgcattgactttgattaagcgtgtgtttggttacccgcacgtggaaggtgcggattgtacgagccgatgcaaaacaagccatttagaggccaattgactgcatccgctcgtaccgccgaaatgccggtatctggccagccaggctcaggacagcctgtagctacggacacacttaaccaatcagggccagtatgaaccgttggatgctctgcatgagcatgatggagtaagtagaactagctagctgtactgtactgtatcccgacaggcacagccacagatgattgacttcttacttgcttgtacgtctagctataaaccgtattaattagttaggtatatttgctaagcatagaaactacgggaaaccacacttttcccgtttccccatcatcgtcatcacttgagaaaaaaaatctagcaacgatgtgacacgtcttattagatagcgctgcttgttaatctttttccattccagctatctagctatgctccttgacgcgcactgctacgtgctacaatatatacatacatacataatatatgtatgtgtatacatgattttaatatatgtagagaaggaggacgacgacgtctaaatataacgccggtgtggggtgtgtgatatatatatattttgcagataactgagacgaaggtcacccgactatgccatgagaagaccatcctggccgtgaacgggcagttcccggggccgaccatctacgcgcgcaaggacgacgtggtcatcgtcaacgtgtacaaccagggctacaagaacatcaccctccactggcacggcgtggaccagccgcggaacccgtggtccgatggcccggagtacatcacgcagtgccccatccagcccggcgccaacttcacctacaagatcatcttcaccgaggaggaaggcacgctgtggtggcacgcgcacagcgaattcgaccgcgccaccgtgcacggcgccatcgtcatccaccccaagcgcggcaccgtctacccctaccccaagccgcacaaggagatgcccatcatcctcggcgagtggtggaacgcggacgtggagcagatcctcctcgagtcccagcggaccggcggcgacgtcaacatttcggacgccaacaccatcaacggccagcccggcgacttcgccccgtgctccaaggaggacaccttcaagatgtccgtggagcacggcaagacgtacctgctccgggtcatcaacgcggggctcaccaacgagatgttcttcgccgtcgccgggcaccgcctcacggtggtcggcaccgacggccgctacctcaggccgttcaccgtcgactacatcctcatctcccccggacagaccatgaacatgctcctcgaggccaactgcgccaccgacggctcagccaacagccgctactacatggctgcgaggccgttcttcaccaacacggcagtcaatgtcgacgacaaaaacaccacggccattctggagtacacggacgcgccaccctccgcggggccaccggactcccccgacctgccggccatggacgacatcgccgcggcgacggcgtacacggcgcagctccggtccctggtcaccaaggagcatccgatcgacgtgccgatggaggtggacgagcacatgctcgtgacgatctccgtcaacacgatcccctgcgagcccaacaagacgtgcgccggccccggaaacaaccgcctcgccgcgagcctgaacaacgtcagcttcatgaacccgaccatcgacatcctcgacgcctactacgactccatcagcggcgtgtacgagccggacttccccaacaagccgcccttcttcttcaacttcaccgctcccaacccgccacaggacctctggttcacgaagcggggcaccaaggtgaaggtggtggagtacggcaccgtcctggaggtggtgttccaggacacggccatcctcggcgccgagagccaccccatgcacctgcacggcttcagcttctacgtggtgggccgaggcttcggtaacttcgacaaggacaaggaccccgccacgtacaacctggtcgacccgccgtaccagaacaccgtctccgtgcccacgggcggttgggctgcaatgcgcttccgagcggcaaatcctggtgagtactttatttactaaattaattaaaatttgggcaacaatatatatatatatatatatatatatattgttctatggattttattttcttatatatgtgtgctgttttttcttatatttttttaggtgtgtggtttatgcattgccactttgatcgtcacacggtgtggggcatggacactgtgttcattgtgaaaaatggcaagggcccggacgctcagatgatgccacgtccccctaacatgcccaagtgctgagaaaacaagggcacgagctacgactgctcgggttgcatgcaaggcgctcgatcaaaccagctaatcttagttgattggttgatttaattatttgtggtacatattttaagtagaacggttcttcaaataaaacggccagttgagatgtaattagtgtcatttgtgttcttttctctttttattcatttgattgtaagagaaaaacaaattcattatatttattatttgtgtcggtctactgctagttcaatctccaagtgtaattaaacaatgtatgtcaaatcatgtatctagtgaaaattcaatataaatgcgtgcttcatatgtgtatttattt

SEQ ID NO: 3 - DNA, 1773 bp, Zea mays
atgccccttcgacaacgtccgacgatgggcggcggcggcggcggtgtagctaagatgccggcaggccagctctggttattactgctaggcgtgttgttgttagcatttggagtcccagcccaggcctccaggaatactcactacgacttcgttataactgagacgaaggtcacccgactatgccatgagaagaccatcctggccgtgaacgggcagttcccggggccgaccatctacgcgcgcaaggacgacgtggtcatcgtcaacgtgtacaaccagggctacaagaacatcaccctccactggcacggcgtggaccagccgcggaacccgtggtccgatggcccggagtacatcacgcagtgccccatccagcccggcgccaacttcacctacaagatcatcttcaccgaggaggaaggcacgctgtggtggcacgcgcacagcgaattcgaccgcgccaccgtgcacggcgccatcgtcatccaccccaagcgcggcaccgtctacccctaccccaagccgcacaaggagatgcccatcatcctcggcgagtggtggaacgcggacgtggagcagatcctcctcgagtcccagcggaccggcggcgacgtcaacatttcggacgccaacaccatcaacggccagcccggcgacttcgccccgtgctccaaggaggacaccttcaagatgtccgtggagcacggcaagacgtacctgctccgggtcatcaacgcggggctcaccaacgagatgttcttcgccgtcgccgggcaccgcctcacggtggtcggcaccgacggccgctacctcaggccgttcaccgtcgactacatcctcatctcccccggacagaccatgaacatgctcctcgaggccaactgcgccaccgacggctcagccaacagccgctactacatggctgcgaggccgttcttcaccaacacggcagtcaatgtcgacgacaaaaacaccacggccattctggagtacacggacgcgccaccctccgcggggccaccggactcccccgacctgccggccatggacgacatcgccgcggcgacggcgtacacggcgcagctccggtccctggtcaccaaggagcatccgatcgacgtgccgatggaggtggacgagcacatgctcgtgacgatctccgtcaacacgatcccctgcgagcccaacaagacgtgcgccggccccggaaacaaccgcctcgccgcgagcctgaacaacgtcagcttcatgaacccgaccatcgacatcctcgacgcctactacgactccatcagcggcgtgtacgagccggacttccccaacaagccgcccttcttcttcaacttcaccgctcccaacccgccacaggacctctggttcacgaagcggggcaccaaggtgaaggtggtggagtacggcaccgtcctggaggtggtgttccaggacacggccatcctcggcgccgagagccaccccatgcacctgcacggcttcagcttctacgtggtgggccgaggcttcggtaacttcgacaaggacaaggaccccgccacgtacaacctggtcgacccgccgtaccagaacaccgtctccgtgcccacgggcggttgggctgcaatgcgcttccgagcggcaaatcctggtgtgtggtttatgcattgccactttgatcgtcacacggtgtggggcatggacactgtgttcattgtgaaaaatggcaagggcccggacgctcagatgatgccacgtccccctaacatgcccaagtgctga

SEQ ID NO: 4 - protein, 582 aa, Zea mays
MGARRGLRRGQAAAAAFSACPFLALAVVLLALPELAAGDTHYYTFNVQMTNVTRLCVTKSIPTVNGEFPGPKLVVREGDRLVVKVHNHINYNVSFHWHGVRQLRNGWADGPSYITQCPIQGGQSYVYDFTVTGQRGTLWWHAHFSWLRVHLYGPLVILPKRGEGYPFPRPYKEVPILFGEWFNADTEAVINQALQTGAGPNVSDAYTFNGLPGPTYNCSSKDTYKLKVKPGRTYMLRLINSALNDELFFGIANHTLTVVEADASYVKPFTVSTLVISPGQTMNVLLTTAPSPASPAYAMAIAPYTNTQGTFDNTTAAAVLEYAPTTTRNNTLPPLPALPLYNDTGAVSNFSRNFRSLNSARYPARVPVAVDRHLLFTVGLGTDPCPYTNQTCQGPNGTKFAASVNNNSFFRPRTALLEAHYRRRYAGVLLADFPTAPPHPFNYTGTPPNNTFVQHGTRVVPLRFNASVELVLQGTSIQGAESHPLHLHGYNFFVVGQGFGNFDPVNDPPGYNLADPVERNTISVPTAGWVAVRFLADNPGVWLMHCHFDVHLSWGLSMAWLVNDGPLPNEKMLPPPSDLPKC

SEQ ID NO: 5 - DNA, 2771 bp, Zea mays
caccaactaggccaaccaccaccgtgctgtgaccccctaccatgcaggccacgaacccggcggccatcatggcgcctatatagaacccagcactcattccatagcaaagtgcaccacttcacttgcttcaaagcgcaaacacacaagaagggcggagctgttgtcatcctgacaatgggcgcgcgtcgtggtctccggcgaggccaagccgccgccgccgccttctccgcatgtcccttcctcgccctcgccgtcgtcctcctcgccttgccggagctcgcagccggcgacacccactactacacgttcaacgtaaaactactctagcgcacgagcattacaagatccagcgcgcacacacacacatcgtcatcatcatcatcagtgtttctcactattactggccggtatggatttggttcgtgctgcaggtgcaaatgaccaacgtgacacggctgtgcgtgactaagagcatcccgacggtgaacggggagttcccggggccgaagctggtcgtgcgggaaggcgaccgcctcgtggtcaaggttcacaaccacatcaactacaatgtctcgttccactggtaagtgggatcgtatcacagcagcaagcgcaccgcgccgtgttgccatctctctgccagccggccggcgtctcatcacccgcgtcgtcacgcaggcacggcgtccggcagctgcgcaacgggtgggcggacgggccgtcgtacatcacgcagtgcccgatccagggcgggcagagctacgtgtacgacttcaccgtcacggggcagcgcggcacgctgtggtggcacgcgcacttctcctggctgcgcgtgcacctctacggcccgctcgtcatcctccccaagcgcggcgagggctacccgttcccgcgcccctacaaggaggtgcccatcctcttcggtacaacgcctgcccgctgctcgcgctcgtcgtcttccttccatcgatcgcttgatggagttccgtgcgtattgcgtggattctttatttgcaggcgaatggttcaacgcggacacggaggccgtcatcaaccaggccctgcaaacaggcgccggcccaaacgtctccgatgcctacaccttcaatgggcttccaggcccgacatataactgctcgtctaaaggtacgctttctttgcaagggctagattatttaggcccatcgcttgtactttttatttagtcatgttattagaaagataaaacgagatgaagataacaaaagcctggatttgtgatttctttttggatgaaacaaaaaaccagacacgtacaagctgaaggtgaagcccgggaggacgtacatgctccggctcatcaactccgccctcaacgacgagctcttcttcggcatcgccaaccacacgctcaccgtcgtcgaggcggacgccagctacgtcaagccattcaccgtcagcacgctcgtcatttcaccggggcagaccatgaacgtgctcctcacgacggcccccagccccgcctccccggcctacgccatggcgatcgcgccctacaccaacacgcagggcacgttcgacaacaccaccgccgcggccgtcctcgagtacgccccgacgacgaccaggaacaacaccctgcctcccctaccggccctgccgctgtacaacgacaccggcgcggtgtccaacttctcgcgcaatttccgcagcctgaacagcgcgcgctacccggcgcgcgtgccggtggcggtggaccggcacctgctgttcaccgtggggctcggcacggacccgtgcccgtacaccaaccagacgtgccagggccccaacggcaccaagttcgcggcgtccgtcaacaacaactccttcttccgcccccggaccgcgctcctcgaggcgcactaccggcgccgctacgccggcgtgctcctggccgacttccccacggccccgccgcacccgttcaactacacgggcaccccgcccaacaacacgttcgtgcagcacggcacgcgggtggtgccgctccgcttcaacgcctccgtggagctggtgctgcagggcaccagcatccagggcgccgagagccacccgctgcacctgcacggctacaacttcttcgtggtcggccaagggttcggcaacttcgacccggtgaacgacccgcccgggtacaacctcgccgaccccgtagagcgcaacaccatcagcgtgcccaccgccggctgggtcgccgtccggttcctcgccgacaacccgggtaatcaatcaactgatccatgcacatttatcatgctctgcctgtgttgacttggttgcatatatctggcaggcgtgtggctgatgcattgccacttcgacgtgcacttgagctggggcctgtccatggcgtggcttgtcaacgacggcccgctgccgaacgagaagatgttgcccccgccatccgacctcccaaaatgctgatgacgactggtcgtttatcacccgatcgaggggtagatgggcatttaggaaggttctcctgcttcctgcacgtctgcctacttcctttccttacgatgtttggaactatttggtttggactatttaattaccgtgtgccgatttttggcgagtgcttggatttcgcgatcctcgctgaatccccttttgaaacatgttaatctgtatctatgtaacgacaacgtttgttctgcggttacttgttctttttttaccccctttctgaacattcagcacgcattggtgtattcacatggtcaaatacaatgtaacaatgatgtctgtat

SEQ ID NO: 6 - DNA, 1749 bp, Zea mays
atgggcgcgcgtcgtggtctccggcgaggccaagccgccgccgccgccttctccgcatgtcccttcctcgccctcgccgtcgtcctcctcgccttgccggagctcgcagccggcgacacccactactacacgttcaacgtgcaaatgaccaacgtgacacggctgtgcgtgactaagagcatcccgacggtgaacggggagttcccggggccgaagctggtcgtgcgggaaggcgaccgcctcgtggtcaaggttcacaaccacatcaactacaatgtctcgttccactggcacggcgtccggcagctgcgcaacgggtgggcggacgggccgtcgtacatcacgcagtgcccgatccagggcgggcagagctacgtgtacgacttcaccgtcacggggcagcgcggcacgctgtggtggcacgcgcacttctcctggctgcgcgtgcacctctacggcccgctcgtcatcctccccaagcgcggcgagggctacccgttcccgcgcccctacaaggaggtgcccatcctcttcggcgaatggttcaacgcggacacggaggccgtcatcaaccaggccctgcaaacaggcgccggcccaaacgtctccgatgcctacaccttcaatgggcttccaggcccgacatataactgctcgtctaaagacacgtacaagctgaaggtgaagcccgggaggacgtacatgctccggctcatcaactccgccctcaacgacgagctcttcttcggcatcgccaaccacacgctcaccgtcgtcgaggcggacgccagctacgtcaagccattcaccgtcagcacgctcgtcatttcaccggggcagaccatgaacgtgctcctcacgacggcccccagccccgcctccccggcctacgccatggcgatcgcgccctacaccaacacgcagggcacgttcgacaacaccaccgccgcggccgtcctcgagtacgccccgacgacgaccaggaacaacaccctgcctcccctaccggccctgccgctgtacaacgacaccggcgcggtgtccaacttctcgcgcaatttccgcagcctgaacagcgcgcgctacccggcgcgcgtgccggtggcggtggaccggcacctgctgttcaccgtggggctcggcacggacccgtgcccgtacaccaaccagacgtgccagggccccaacggcaccaagttcgcggcgtccgtcaacaacaactccttcttccgcccccggaccgcgctcctcgaggcgcactaccggcgccgctacgccggcgtgctcctggccgacttccccacggccccgccgcacccgttcaactacacgggcaccccgcccaacaacacgttcgtgcagcacggcacgcgggtggtgccgctccgcttcaacgcctccgtggagctggtgctgcagggcaccagcatccagggcgccgagagccacccgctgcacctgcacggctacaacttcttcgtggtcggccaagggttcggcaacttcgacccggtgaacgacccgcccgggtacaacctcgccgaccccgtagagcgcaacaccatcagcgtgcccaccgccggctgggtcgccgtccggttcctcgccgacaacccgggcgtgtggctgatgcattgccacttcgacgtgcacttgagctggggcctgtccatggcgtggcttgtcaacgacggcccgctgccgaacgagaagatgttgcccccgccatccgacctcccaaaatgctga

SEQ ID NO: 7 - DNA, 524 bp, Artificial Sequence
  RNA inhibitor
aagaaaaatggccatcccctagctaggtgaagaagaatgaaaacctctaatttatctagaggttattcatcttttaggggatggcctaaatacaaaatgaaaactctctaattaagtggttttgtgttcatgtaaggaaagcgttttaagatatggagcaatgaagactgcagaaggctgattcagactgcgagttttgtttatctccctctagaaactcctctgcatctagccccttccaagcttcggttcccctcggaatcagcagattatgtatctttaattttgtaatactctctctcttctctatgctttgtttttcttcattatgtttgggttgtacccactcccgcgcgttgtgtgttctttgtgtgaggaataaaaaaatattcggatttgagaactaaaactagagtagttttattgatattcttgtttttcatttagtatctaataagtttggagaatagtcagaccagtgcatgtaaatttgcttccgattctctttatagtgaattcctctt

SEQ ID NO: 8 - RNA, 524 nt, Artificial Sequence
  RNA inhibitor
aagaaaaauggccauccccuagcuaggugaagaagaaugaaaaccucuaauuuaucuagagguuauucaucuuuuaggggauggccuaaauacaaaaugaaaacucucuaauuaagugguuuuguguucauguaaggaaagcguuuuaagauauggagcaaugaagacugcagaaggcugauucagacugcgaguuuuguuuaucucccucuagaaacuccucugcaucuagccccuuccaagcuucgguuccccucggaaucagcagauuauguaucuuuaauuuuguaauacucucucucuucucuaugcuuuguuuuucuucauuauguuuggguuguacccacucccgcgcguuguguguucuuugugugaggaauaaaaaaauauucggauuugagaacuaaaacuagaguaguuuuauugauauucuuguuuuucauuuaguaucuaauaaguuuggagaauagucagaccagugcauguaaauuugcuuccgauucucuuuauagugaauuccucuu

SEQ ID NO: 9 - RNA, 21 nt, Zea mays
uggaaggggcaugcagaggag

SEQ ID NO: 10 - DNA, 21 bp, Zea mays
tggaaggggcatgcagaggag

SEQ ID NO: 11 - DNA, 12103 bp, Artificial Sequence
  Plasmid/vector sequence (pCUB)
ctcattaggcaccccaggctttacactttatgcttccggctcgtatgttgtgtggaattgtgagcggataacaatttcacacaggaaacagctatgacatgattacgaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctcggtacccggggatcctctagagtcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagagggcacgaccgggtcacgctgcactgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctggtgggctatgcccgcgtcagcaccgacgaccaggacttgaccaaccaacgggccgaactgcacgcggcctatgaggtaaagagaaaatgagcaaaagccaaacacgctaagtgccggccgtccgagcgcacgcagcagcaaggctgcaacgttggccagcctggcagacacgccagccatgaagcgggtcaactttcagttgccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggctgatgaatcccctaatgattttggtaaaaatcattaagttaaggtggatacacatcttgtcatatgatcaaatggtttcgcgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctccacaaagagcgactcgctgtataccgttggcatgctagctttatctgttcgggcaatacgatgcccattgtacttgttgactggtctgatattcgtgagcaaaaacgacttatggtattgcgagcttcagtcgcactacacggtcgttctgttactctttatgagaaagcgttcccgctttcagagcaatgttcaaagaaagctcatgaccaatttctagccgaccttgcgagcattctaccgagtaacaccacaccgctcattgtcagtgatgctggctttaaagtgccatggtataaatccgttgagaagctgggttggtactggttaagtcgagtaagaggaaaagtacaatatgcagacctaggagcggaaaactggaaacctatcagcaacttacatgatatgtcatctagtcactcaaagactttaggctataagaggctgactaaaagcaatccaatctcatgccaaattctattgtataaatctcgctctaaaggccgaaaaaatcagcgctcgacacggactcattgtcaccacccgtcacctaaaatctactcagcgtcggcaaaggagccatgggttctagcaactaacttacctgttgaaattcgaacacccaaacaacttgttaatatctattcgaagcgaatgcagattgaagaaaccttccgagacttgaaaagtcctgcctacggactaggcctacgccatagccgaacgagcagctcagagcgttttgatatcatgctgctaatcgccctgatgcttcaactaacatgttggcttgcgggcgttcatgctcagaaacaaggttgggacaagcacttccaggctaacacagtcagaaatcgaaacgtactctcaacagttcgcttaggcatggaagttttgcggcattctggctacacaataacaagggaagacttactcgtggctgcaaccctactagctcaaaatttattcacacatggttacgctttggggaaattatgaggggatctctcagcgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagactcgagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctca

SEQ ID NO: 12 - DNA, 1803 bp, Artificial Sequence
  PCR amplification sequence
gactctagaggatccatgccccttcgacaacgtccgacgatgggcggcggcggcggcggtgtagctaagatgccggcaggccagctctggttattactgctaggcgtgttgttgttagcatttggagtcccagcccaggcctccaggaatactcactacgacttcgttataactgagacgaaggtcacccgactatgccatgagaagaccatcctggccgtgaacgggcagttcccggggccgaccatctacgcgcgcaaggacgacgtggtcatcgtcaacgtgtacaaccagggctacaagaacatcaccctccactggcacggcgtggaccagccgcggaacccgtggtccgatggcccggagtacatcacgcagtgccccatccagcccggcgccaacttcacctacaagatcatcttcaccgaggaggaaggcacgctgtggtggcacgcgcacagcgaattcgaccgcgccaccgtgcacggcgccatcgtcatccaccccaagcgcggcaccgtctacccctaccccaagccgcacaaggagatgcccatcatcctcggcgagtggtggaacgcggacgtggagcagatcctcctcgagtcccagcggaccggcggcgacgtcaacatttcggacgccaacaccatcaacggccagcccggcgacttcgccccgtgctccaaggaggacaccttcaagatgtccgtggagcacggcaagacgtacctgctccgggtcatcaacgcggggctcaccaacgagatgttcttcgccgtcgccgggcaccgcctcacggtggtcggcaccgacggccgctacctcaggccgttcaccgtcgactacatcctcatctcccccggacagaccatgaacatgctcctcgaggccaactgcgccaccgacggctcagccaacagccgctactacatggctgcgaggccgttcttcaccaacacggcagtcaatgtcgacgacaaaaacaccacggccattctggagtacacggacgcgccaccctccgcggggccaccggactcccccgacctgccggccatggacgacatcgccgcggcgacggcgtacacggcgcagctccggtccctggtcaccaaggagcatccgatcgacgtgccgatggaggtggacgagcacatgctcgtgacgatctccgtcaacacgatcccctgcgagcccaacaagacgtgcgccggccccggaaacaaccgcctcgccgcgagcctgaacaacgtcagcttcatgaacccgaccatcgacatcctcgacgcctactacgactccatcagcggcgtgtacgagccggacttccccaacaagccgcccttcttcttcaacttcaccgctcccaacccgccacaggacctctggttcacgaagcggggcaccaaggtgaaggtggtggagtacggcaccgtcctggaggtggtgttccaggacacggccatcctcggcgccgagagccaccccatgcacctgcacggcttcagcttctacgtggtgggccgaggcttcggtaacttcgacaaggacaaggaccccgccacgtacaacctggtcgacccgccgtaccagaacaccgtctccgtgcccacgggcggttgggctgcaatgcgcttccgagcggcaaatcctggtgtgtggtttatgcattgccactttgatcgtcacacggtgtggggcatggacactgtgttcattgtgaaaaatggcaagggcccggacgctcagatgatgccacgtccccctaacatgcccaagtgctgaggatccccgggtacc

SEQ ID NO: 13 - DNA, 78 bp, Artificial Sequence
  DNA molecule I
gtggaaggggcatgcagaggagcacgaacgaggtgtggttggcccctcgttagctctcctgtgcctgcctcttccatt

SEQ ID NO: 14 - RNA, 78 nt, Artificial Sequence
  Precursor RNA of miRNA
guggaaggggcaugcagaggagcacgaacgaggugugguuggccccucguuagcucuccugugccugccucuuccauu

SEQ ID NO: 15 - RNA, 21 nt, Artificial Sequence
  miRNA
uggaaggggcaugcagaggag

SEQ ID NO: 16 - DNA, 2249 bp, Zea mays
caccaactaggccaaccaccaccgtgctgtgaccccctaccatgcaggccacgaacccggcggccatcatggcgcctatatagaacccagcactcattccatagcaaagtgcaccacttcacttgcttcaaagcgcaaacacacaagaagggcggagctgttgtcatcctgacaatgggcgcgcgtcgtggtctccggcgaggccaagccgccgccgccgccttctccgcatgtcccttcctcgccctcgccgtcgtcctcctcgccttgccggagctcgcagccggcgacacccactactacacgttcaacgtgcaaatgaccaacgtgacacggctgtgcgtgactaagagcatcccgacggtgaacggggagttcccggggccgaagctggtcgtgcgggaaggcgaccgcctcgtggtcaaggttcacaaccacatcaactacaatgtctcgttccactggcacggcgtccggcagctgcgcaacgggtgggcggacgggccgtcgtacatcacgcagtgcccgatccagggcgggcagagctacgtgtacgacttcaccgtcacggggcagcgcggcacgctgtggtggcacgcgcacttctcctggctgcgcgtgcacctctacggcccgctcgtcatcctccccaagcgcggcgagggctacccgttcccgcgcccctacaaggaggtgcccatcctcttcggcgaatggttcaacgcggacacggaggccgtcatcaaccaggccctgcaaacaggcgccggcccaaacgtctccgatgcctacaccttcaatgggcttccaggcccgacatataactgctcgtctaaagacacgtacaagctgaaggtgaagcccgggaggacgtacatgctccggctcatcaactccgccctcaacgacgagctcttcttcggcatcgccaaccacacgctcaccgtcgtcgaggcggacgccagctacgtcaagccattcaccgtcagcacgctcgtcatttcaccggggcagaccatgaacgtgctcctcacgacggcccccagccccgcctccccggcctacgccatggcgatcgcgccctacaccaacacgcagggcacgttcgacaacaccaccgccgcggccgtcctcgagtacgccccgacgacgaccaggaacaacaccctgcctcccctaccggccctgccgctgtacaacgacaccggcgcggtgtccaacttctcgcgcaatttccgcagcctgaacagcgcgcgctacccggcgcgcgtgccggtggcggtggaccggcacctgctgttcaccgtggggctcggcacggacccgtgcccgtacaccaaccagacgtgccagggccccaacggcaccaagttcgcggcgtccgtcaacaacaactccttcttccgcccccggaccgcgctcctcgaggcgcactaccggcgccgctacgccggcgtgctcctggccgacttccccacggccccgccgcacccgttcaactacacgggcaccccgcccaacaacacgttcgtgcagcacggcacgcgggtggtgccgctccgcttcaacgcctccgtggagctggtgctgcagggcaccagcatccagggcgccgagagccacccgctgcacctgcacggctacaacttcttcgtggtcggccaagggttcggcaacttcgacccggtgaacgacccgcccgggtacaacctcgccgaccccgtagagcgcaacaccatcagcgtgcccaccgccggctgggtcgccgtccggttcctcgccgacaacccgggcgtgtggctgatgcattgccacttcgacgtgcacttgagctggggcctgtccatggcgtggcttgtcaacgacggcccgctgccgaacgagaagatgttgcccccgccatccgacctcccaaaatgctgatgacgactggtcgtttatcacccgatcgaggggtagatgggcatttaggaaggttctcctgcttcctgcacgtctgcctacttcctttccttacgatgtttggaactatttggtttggactatttaattaccgtgtgccgatttttggcgagtgcttggatttcgcgatcctcgctgaatccccttttgaaacatgttaatctgtatctatgtaacgacaacgtttgttctgcggttacttgttctttttttaccccctttctgaacattcagcacgcattggtgtattcacatggtcaaatacaatgtaacaatgatgtctgtat

SEQ ID NO: 17 - DNA, 2420 bp, Zea mays
accgactggtggcggcatgacgaacgaaacatgcatatgcattcgtcccctcgtcgtcgttggcagctctcgctcctctataaataccagcgccatccgcttcagatgagcatcgatcccagcaacgcacggagcgtacgtacattgcagtagctagctatagctggccggccatcccctctcgctcgctgctaaacacgttccagcttgtttgctcagagaaacagcgcgcgcgcacacacacacacatcatcatcatcgattcatcgtacacaggatcagagagcttaattagttctagctctgctgcatgccccttcgacaacgtccgacgatgggcggcggcggcggcggtgtagctaagatgccggcaggccagctctggttattactgctaggcgtgttgttgttagcatttggagtcccagcccaggcctccaggaatactcactacgacttcgttataactgagacgaaggtcacccgactatgccatgagaagaccatcctggccgtgaacgggcagttcccggggccgaccatctacgcgcgcaaggacgacgtggtcatcgtcaacgtgtacaaccagggctacaagaacatcaccctccactggcacggcgtggaccagccgcggaacccgtggtccgatggcccggagtacatcacgcagtgccccatccagcccggcgccaacttcacctacaagatcatcttcaccgaggaggaaggcacgctgtggtggcacgcgcacagcgaattcgaccgcgccaccgtgcacggcgccatcgtcatccaccccaagcgcggcaccgtctacccctaccccaagccgcacaaggagatgcccatcatcctcggcgagtggtggaacgcggacgtggagcagatcctcctcgagtcccagcggaccggcggcgacgtcaacatttcggacgccaacaccatcaacggccagcccggcgacttcgccccgtgctccaaggaggacaccttcaagatgtccgtggagcacggcaagacgtacctgctccgggtcatcaacgcggggctcaccaacgagatgttcttcgccgtcgccgggcaccgcctcacggtggtcggcaccgacggccgctacctcaggccgttcaccgtcgactacatcctcatctcccccggacagaccatgaacatgctcctcgaggccaactgcgccaccgacggctcagccaacagccgctactacatggctgcgaggccgttcttcaccaacacggcagtcaatgtcgacgacaaaaacaccacggccattctggagtacacggacgcgccaccctccgcggggccaccggactcccccgacctgccggccatggacgacatcgccgcggcgacggcgtacacggcgcagctccggtccctggtcaccaaggagcatccgatcgacgtgccgatggaggtggacgagcacatgctcgtgacgatctccgtcaacacgatcccctgcgagcccaacaagacgtgcgccggccccggaaacaaccgcctcgccgcgagcctgaacaacgtcagcttcatgaacccgaccatcgacatcctcgacgcctactacgactccatcagcggcgtgtacgagccggacttccccaacaagccgcccttcttcttcaacttcaccgctcccaacccgccacaggacctctggttcacgaagcggggcaccaaggtgaaggtggtggagtacggcaccgtcctggaggtggtgttccaggacacggccatcctcggcgccgagagccaccccatgcacctgcacggcttcagcttctacgtggtgggccgaggcttcggtaacttcgacaaggacaaggaccccgccacgtacaacctggtcgacccgccgtaccagaacaccgtctccgtgcccacgggcggttgggctgcaatgcgcttccgagcggcaaatcctggtgtgtggtttatgcattgccactttgatcgtcacacggtgtggggcatggacactgtgttcattgtgaaaaatggcaagggcccggacgctcagatgatgccacgtccccctaacatgcccaagtgctgagaaaacaagggcacgagctacgactgctcgggttgcatgcaaggcgctcgatcaaaccagctaatcttagttgattggttgatttaattatttgtggtacatattttaagtagaacggttcttcaaataaaacggccagttgagatgtaattagtgtcatttgtgttcttttctctttttattcatttgattgtaagagaaaaacaaattcattatatttattatttgtgtcggtctactgctagttcaatctccaagtgtaattaaacaatgtatgtcaaatcatgtatctagtgaaaattcaatataaatgcgtgcttcatatgtgtatttattt

SEQ ID NO: 18 - DNA, 205 bp, Zea mays
cgctcgatcaaaccagctaatcttagttgattggttgatttaattatttgtggtacatattttaagtagaacggttcttcaaataaaacggccagttgagatgtaattagtgtcatttgtgttcttttctctttttattcatttgattgtaagagaaaaacaaattcattatatttattatttgtgtcggtctactgctagttca

SEQ ID NO: 19 - DNA, 171 bp, Zea mays
tttatcacccgatcgaggggtagatgggcatttaggaaggttctcctgcttcctgcacgtctgcctacttcctttccttacgatgtttggaactatttggtttggactatttaattaccgtgtgccgatttttggcgagtgcttggatttcgcgatcctcgctgaatcccc

SEQ ID NO: 20 - RNA, 21 nt, Zea mays
cucugcugcaugccccuucga

SEQ ID NO: 21 - RNA, 21 nt, Zea mays
cuucuccgcaugucccuuccu

SEQ ID NO: 22 - DNA, 34 bp, Artificial Sequence
  Primer
gactctagaggatccatgccccttcgacaacgtc

SEQ ID NO: 23 - DNA, 36 bp, Artificial Sequence
  Primer
ggtacccggggatcctcagcacttgggcatgttagg

SEQ ID NO: 24 - DNA, 22 bp, Artificial Sequence
  Primer
gcgtgttgttgttagcatttgg

SEQ ID NO: 25 - DNA, 22 bp, Artificial Sequence
  Primer
gggtgatgttcttgtagccctg

SEQ ID NO: 26 - DNA, 21 bp, Artificial Sequence
  Primer
gctgccgatgtgcctgcgtcg

SEQ ID NO: 27 - DNA, 27 bp, Artificial Sequence
  Primer
ctgaaagacagaacataatgagcacag

SEQ ID NO: 28 - DNA, 50 bp, Artificial Sequence
  Primer
gtcgtatccagtgcagggtccgaggtattcgcactggatacgacctcctc

SEQ ID NO: 29 - DNA, 16 bp, Artificial Sequence
  Primer
gtggaaggggcatgca

SEQ ID NO: 30 - DNA, 16 bp, Artificial Sequence
  Primer
gtgcagggtccgaggt

SEQ ID NO: 31 - DNA, 78 bp, Artificial Sequence
  CRISPR sequence
gggcgaaacaacacagcgagttaaaataaggcttagtccgtactcaacttgaaaaggtggcaccgattcggtgttttt

SEQ ID NO: 32 - DNA, 123 bp, Artificial Sequence
  RNA inhibitor
gtggaaggggcatgcagaggaggagcacgagcgaggtgtggctggaagaagcagccggggcctgtgtgctatatacccctcgcaagctctcctcactcctctcctgtgcctgcctcttccatt

SEQ ID NO: 33 - DNA, 23 bp, Zea mays
tcagggtcagtttgctctgctgg

SEQ ID NO: 34 - DNA, 20 bp, Artificial Sequence
  CRISPR construct
tcagggtcagtttgctctgc

SEQ ID NO: 35 - DNA, 96 bp, Artificial Sequence
  CRISPR construct
tcagggtcagtttgctctgcgttttagagctagaaatagcaagttaaaataaggctagtccgttatcaacttgaaaaagtggcaccgagtcggtgc

SEQ ID NO: 36 - DNA, 23 bp, Zea mays
tgcgcagttgccctgtgatgagg

SEQ ID NO: 37 - DNA, 20 bp, Artificial Sequence
  CRISPR construct
tgcgcagttgccctgtgatg

SEQ ID NO: 38 - DNA, 96 bp, Artificial Sequence
  CRISPR construct
tgcgcagttgccctgtgatggttttagagctagaaatagcaagttaaaataaggctagtccgttatcaacttgaaaaagtggcaccgagtcggtgc

SEQ ID NO: 39 - DNA, 78 bp, Artificial Sequence; identical to SEQ ID NO: 13
  RNA inhibitor

SEQ ID NO: 40 - DNA, 23 bp, Zea mays
tctctagttctgggagttcctgg

SEQ ID NO: 41 - DNA, 20 bp, Artificial Sequence
  CRISPR construct
tctctagttctgggagttcc

SEQ ID NO: 42 - DNA, 96 bp, Artificial Sequence
  CRISPR construct
tctctagttctgggagttccgttttagagctagaaatagcaagttaaaataaggctagtccgttatcaacttgaaaaagtggcaccgagtcggtgc

SEQ ID NO: 43 - DNA, 23 bp, Zea mays
aaaactcagaagcgcaaccgagg

SEQ ID NO: 44 - DNA, 20 bp, Artificial Sequence
  CRISPR construct
aaaactcagaagcgcaaccg

SEQ ID NO: 45 - DNA, 96 bp, Artificial Sequence
  CRISPR construct
aaaactcagaagcgcaaccggttttagagctagaaatagcaagttaaaataaggctagtccgttatcaacttgaaaaagtggcaccgagtcggtgc

SEQ ID NO: 46 - DNA, 1972 bp, Zea mays
gtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgc

SEQ ID NO: 47 - DNA, 4272 bp, Artificial Sequence
  CRISPR construct
atggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaa

SEQ ID NO: 48 - DNA, 76 bp, Artificial Sequence
  CRISPR construct
gttttagagctagaaatagcaagttaaaataaggctagtccgttatcaacttgaaaaagtggcaccgagtcggtgc

SEQ ID NO: 49 - DNA, 272 bp, Artificial Sequence
  RNA inhibitor
gggattagggataggaatcagggtcagtttgctctgctggctagtagcagcagcggtggaaggggcatgcagaggaggagcacgagcgaggtgtggctggaagaagcagccggggcctgtgtgctatatacccctcgcaagctctcctcactcctctcctgtgcctgcctcttccattccttctgctacgccattatatgtttgcaagcgtcagaagtgcatgcttctgcgcagttgccctgtgatgaggcaaccaacagccaaacatcgct

SEQ ID NO: 50 - DNA, 174 bp, Artificial Sequence
  RNA inhibitor
tcttctcctctctagttctgggagttcctggctgtagcagcagcggtggaaggggcatgcagaggagcacgaacgaggtgtggttggcccctcgttagctctcctgtgcctgcctcttccattccttctgctacgctatgtctgcaagtaaaaaactcagaagcgcaaccgagg

SEQ ID NO: 51 - DNA, 23 bp, Zea mays
atcatcgattcatcgtacacagg

SEQ ID NO: 52 - DNA, 20 bp, Artificial Sequence
  CRISPR construct
atcatcgattcatcgtacac

SEQ ID NO: 53 - DNA, 96 bp, Artificial Sequence
  CRISPR construct
atcatcgattcatcgtacacgttttagagctagaaatagcaagttaaaataaggctagtccgttatcaacttgaaaaagtggcaccgagtcggtgc

SEQ ID NO: 54 - DNA, 23 bp, Zea mays
ccgacgatgggcggcggcggcgg

SEQ ID NO: 55 - DNA, 20 bp, Artificial Sequence
  CRISPR construct
acgatgggcggcggcggcgg

SEQ ID NO: 56 - DNA, 96 bp, Artificial Sequence
  CRISPR construct
acgatgggcggcggcggcgggttttagagctagaaatagcaagttaaaataaggctagtccgttatcaacttgaaaaagtggcaccgagtcggtgc

SEQ ID NO: 57 - DNA, 23 bp, Zea mays
gcgcgtcgtggtctccggcgagg

SEQ ID NO: 58 - DNA, 20 bp, Artificial Sequence
  CRISPR construct
gcgcgtcgtggtctccggcg

SEQ ID NO: 59 - DNA, 96 bp, Artificial Sequence
  CRISPR construct
gcgcgtcgtggtctccggcggttttagagctagaaatagcaagttaaaataaggctagtccgttatcaacttgaaaaagtggcaccgagtcggtgc

SEQ ID NO: 60 - DNA, 23 bp, Zea mays
ccgcctcgccttgccggagctcg

SEQ ID NO: 61 - DNA, 20 bp, Artificial Sequence
  CRISPR construct
cctcgccttgccggagctcg

SEQ ID NO: 62 - DNA, 96 bp, Artificial Sequence
  CRISPR construct
cctcgccttgccggagctcggttttagagctagaaatagcaagttaaaataaggctagtccgttatcaacttgaaaaagtggcaccgagtcggtgc

SEQ ID NO: 63 - DNA, 3684 bp, Zea mays
atgtctaagttggaaaaattcaccaactgttactctttgtctaagactttgagattcaaggccatcccagttggtaagacccaagaaaacatcgacaacaagagactattagttgaagatgaaaagagagctgaagactacaagggtgtcaagaaattgttggacagatactacttgtcttttatcaacgacgttttgcattccatcaagctaaagaacttgaataactacatctctttgttcagaaagaagactagaactgaaaaggaaaataaggaattggaaaacttggaaatcaacttgagaaaggaaattgctaaggctttcaagggtaatgaaggttacaagtctttattcaagaaagacatcattgaaaccattttgccagaatttttggatgataaggatgaaattgctttggttaactctttcaacggtttcaccactgctttcactggtttcttcgacaacagagaaaacatgttctccgaggaagctaaatccacttctattgctttcagatgtatcaacgaaaacttgacccgttacatctctaacatggacatttttgaaaaggtcgacgccatctttgacaagcacgaagtccaagaaatcaaggaaaagatcttaaactccgactacgatgtcgaagatttcttcgaaggtgaattcttcaactttgttttaacccaagaaggtatcgatgtctacaacgccattatcggtggttttgtcactgaatctggtgaaaagatcaagggtttgaacgaatacattaacttgtacaaccaaaagaccaaacaaaaattgccaaagttcaagccattgtacaagcaagttttgtctgacagagaatctttgtctttttacggtgaagggtacacctctgacgaagaagtcttggaagtcttcagaaacactttgaacaagaactctgaaatcttctcctccatcaagaagttagaaaagttgttcaagaacttcgatgaatactcttctgctggtatcttcgttaagaacggtccagccatctctaccatttctaaggatatctttggtgaatggaacgtcattagagacaaatggaacgctgaatacgatgacatccatttgaagaaaaaggctgttgtcaccgaaaagtacgaagacgacagaagaaaatccttcaagaagatcggttccttctccttggaacaattacaagaatacgccgatgccgatttgtccgttgtcgaaaaattgaaggaaattattattcaaaaggttgatgaaatttacaaagtttacggttcctctgaaaagttattcgatgctgatttcgtcttggaaaagtctttgaagaagaacgacgctgttgtcgctatcatgaaggacttgttggactctgtcaaatctttcgaaaactatatcaaggccttcttcggtgaaggtaaggaaactaacagagatgaatccttctacggtgactttgtcttggcttacgatattttgttgaaggttgaccacatctacgatgccatcagaaactacgttactcaaaagccatactctaaggacaaattcaagttgtacttccaaaacccacaattcatgggtggttgggataaggacaaggaaactgactacagagctaccattttgagatacggttccaagtactacttggccatcatggacaagaagtacgccaagtgtttgcaaaagattgacaaggacgatgtcaacggtaactacgaaaagattaactacaagttgttgccaggtccaaacaagatgttgccaaaggttttcttctccaaaaagtggatggcttactacaacccatctgaagacatccaaaagatctacaagaacggtactttcaaaaagggtgacatgttcaacttaaacgactgtcacaagttgatcgacttcttcaaggactccatctctagatacccaaaatggtccaacgcttacgatttcaacttctctgaaactgaaaaatacaaggatattgctggtttctaccgtgaagtcgaggaacaaggttataaggtttctttcgaatccgcttctaagaaagaagttgacaaattagtcgaagaaggtaagttgtacatgttccaaatctacaacaaagatttctccgacaagtctcacggtactccaaacttgcacaccatgtacttcaagttgctattcgatgaaaacaaccacggtcaaatcagattgtctggtggtgctgaattgttcatgagacgtgcttctctaaagaaggaagaattagtcgtccacccagctaactctccaattgccaacaagaacccagacaaccctaagaagaccaccactttgtcctacgacgtttacaaggacaagagattctccgaagaccaatacgaattgcacattccaattgctatcaacaagtgtccaaagaacatcttcaagatcaacactgaagtcagagttttgttaaagcacgatgacaacccttacgttattggtatcgaccgtggtgaaagaaatttgttgtacattgttgttgttgacggtaagggtaacatcgttgaacaatactccttgaacgaaatcatcaacaacttcaacggtattagaatcaagactgattaccactctttgttggataagaaggaaaaggaacgttttgaagctcgtcaaaactggacctctattgaaaacatcaaagaattgaaggctggttacatcagtcaagttgtccacaagatctgtgaattggtcgagaagtacgatgccgttattgccttggaagatttgaactctggttttaagaactctcgtgtcaaggttgaaaagcaagtctaccaaaagttcgaaaagatgttaatcgacaaattgaactacatggttgacaagaaatccaacccatgtgctaccggtggtgctttgaaaggttaccaaatcaccaacaaattcgaatctttcaaatctatgtccactcaaaacgggttcatcttctacattccagcttggttgacctccaagatcgacccatctaccggtttcgttaacttgttgaagaccaagtacacttccattgctgattccaagaagttcatctcttctttcgacagaatcatgtacgttccagaagaagacttgttcgaattcgccttggactataagaacttctccagaaccgatgctgactacattaagaaatggaaattgtactcctacggtaacagaatcagaattttcagaaacccaaagaaaaacaacgttttcgattgggaagaagtttgtttgacttctgcctacaaggaattattcaacaaatacggtatcaactaccaacaaggtgatatcagagctttgttgtgtgaacaatctgacaaggctttctactcttccttcatggctttgatgtccttgatgttgcaaatgagaaactccatcactggtagaactgatgtcgacttcctcatttctccagttaagaattctgacggtattttctacgactctagaaattacgaagctcaagaaaacgctattttgccaaagaacgctgatgctaacggtgcttacaatattgctagaaaggttttgtgggctatcggtcaattcaagaaggctgaagacgaaaagctagacaaggtcaagattgctatttctaacaaggaatggttggaatacgctcaaacctccgtcaagcac

SEQ ID NO: 64 - protein, 1228 aa, Zea mays
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 65 - DNA, 63 bp, Artificial Sequence
  RNA inhibitor
cacaggatcagagagcttaattagttctagctctggacgatgccactgcgccaacgtccgacg

SEQ ID NO: 66 - DNA, 66 bp, Artificial Sequence
  RNA inhibitor
gcgaggccaagccgccgccgccgccttutccgcttgtccgtttctcgccctcgccgtcgtcctcct

SEQ ID NO: 67 - protein, 5 aa, Artificial Sequence
  Protein Tag. Poly-Arg
RRRRR

SEQ ID NO: 68 - protein, 6 aa, Artificial Sequence
  Protein Tag. Poly-His
HHHHHH

SEQ ID NO: 69 - protein, 8 aa, Artificial Sequence
  Protein Tag. FLAG
DYKDDDDK

SEQ ID NO: 70 - protein, 8 aa, Artificial Sequence
  Protein Tag. Strep-tag II
WSHPQFEK

SEQ ID NO: 71 - protein, 10 aa, Artificial Sequence
  Protein Tag. c-myc
EQKLISEEDL

SEQ ID NO: 72 - RNA, 96 nt, Artificial Sequence
  RNA sequence of SEQ ID NO 35
ucagggucaguuugcucugcguuuuagagcuagaaauagcaaguuaaaauaaggcuaguccguuaucaacuugaaaaaguggcaccgagucggugc

SEQ ID NO: 73 - RNA, 96 nt, Artificial Sequence
  RNA sequence for SEQ ID NO 38
ugcgcaguugcccugugaugguuuuagagcuagaaauagcaaguuaaaauaaggcuaguccguuaucaacuugaaaaaguggcaccgagucggugc

SEQ ID NO: 74 - RNA, 96 nt, Artificial Sequence
  RNA sequence of SEQ ID NO 42
ucucuaguucugggaguuccguuuuagagcuagaaauagcaaguuaaaauaaggcuaguccguuaucaacuugaaaaaguggcaccgagucggugc

SEQ ID NO: 75 - RNA, 96 nt, Artificial Sequence
  RNA sequence of SEQ ID NO 45
aaaacucagaagcgcaaccgguuuuagagcuagaaauagcaaguuaaaauaaggcuaguccguuaucaacuugaaaaaguggcaccgagucggugc

SEQ ID NO: 76 - RNA, 96 nt, Artificial Sequence
  RNA sequence for SEQ ID NO 53
aucaucgauucaucguacacguuuuagagcuagaaauagcaaguuaaaauaaggcuaguccguuaucaacuugaaaaaguggcaccgagucggugc

SEQ ID NO: 77 - RNA, 96 nt, Artificial Sequence
  RNA sequence for SEQ ID NO 56
acgaugggcggcggcggcggguuuuagagcuagaaauagcaaguuaaaauaaggcuaguccguuaucaacuugaaaaaguggcaccgagucggugc

SEQ ID NO: 78 - RNA, 96 nt, Artificial Sequence
  RNA sequence for SEQ ID NO 59
gcgcgucguggucuccggcgguuuuagagcuagaaauagcaaguuaaaauaaggcuaguccguuaucaacuugaaaaaguggcaccgagucggugc

SEQ ID NO: 79 - RNA, 96 nt, Artificial Sequence
  RNA sequence for SEQ ID NO 62
ccucgccuugccggagcucgguuuuagagcuagaaauagcaaguuaaaauaaggcuaguccguuaucaacuugaaaaaguggcaccgagucggugc

(19) United States
(12) Patent Application Publication
(10) Pub. No.: US 2020/0283786 A1
(43) Pub. Date: Sep. 10, 2020

(54) LODGING RESISTANCE IN PLANTS

(71) Applicant: Institute of Crop Science, Chinese Academy of Agricultural Sciences, Beijing (CN)

(72) Inventors: Wen-Xue LI, Beijing (CN); Qing SUN, Beijing (CN)

(21) Appl. No.: 16/758,559

(22) PCT Filed: Oct. 12, 2018
(86) PCT No.: PCT/CN2018/110033

§ 371 Date: Apr. 23, 2020

(30) Foreign Application Priority Data
Oct. 24, 2017 (CN) 201711001759.1
Oct. 24, 2017 (CN) 201711001789.2

(51) Int. Cl. C12N 15/82

(52) U.S. Cl. CPC C12N 15/8261; C12Y 110/03002; C12N 15/8255; C12N 15/8218

(57) ABSTRACT

The invention relates to methods of altering resistance to lodging in maize, transgenic plants with altered resistance to lodging and methods for making such plants. In particular, the invention comprises altering the expression or levels of at least one laccase gene and/or altering the expression or activity of miR528.

### FIELD OF THE INVENTION

The invention relates to methods of altering resistance to lodging in maize, transgenic plants with altered resistance to lodging and methods for making such plants.

### BACKGROUND TO THE INVENTION

Corn (or maize) is one of the most important food crops in the world, and is now consumed by about one third of the world's population as the main food. Corn has a protein content higher than that in rice, a fat content higher than that in flour, rice and millet, and a caloric content higher than that in flour, rice and sorghum. As such in many developed regions, corn is an indispensable food. With the development of the corn processing industry, the quality of corn for consumption has been continuously improving, and new corn foods such as corn flakes, cornmeal, corn grit, special corn flour, and instant corn, etc have been produced, which can be further processed into noodles, bread, biscuits, and so on. Corn can also be processed into corn protein, corn oil, monosodium glutamate, soy sauce, and white wine, etc. Corn is also the king of feed. It is reported that the feed value of 100 kg of corn is equivalent to 135 kg of oats, 120 kg of sorghum or 150 kg of Indica rice. The by-product corn stover or indeed the whole plant (ear/cob plus stover) can also be made into silage. About 65-70% of corn in the world and up to 80% in the developed countries is used as feed, so corn is an important base for the development of the livestock industry.

To obtain high yields, farmers often apply excessive quantities of chemical nitrogen (N) fertilizer. For example, in China synthetic N fertilizer application increased from 7.07 million tons in 1977 to 26.21 million tons in 2005 (Ju et al., 2009). These large inputs of external N combined with decreasing N-use efficiency results in environmental problems (Ju et al., 2009; Guo et al., 2010; Liu et al., 2013) and also in yield losses due to lodging under “N-luxury” conditions (Zhang et al., 2016; Shah et al., 2017; Khan et al., 2018). Excessive N fertilizer application exacerbates poor lodging resistance by increasing the height of the crop center of gravity and by decreasing the stem diameter and the cell wall thickness of basal internodes (Wang et al., 2012; Zhang et al., 2014). However, the molecular mechanisms regulating lodging remain to be explored.

Cereal plant lodging includes root lodging and stem lodging (Zhang et al., 2016). Stem strength plays an important role in stem lodging resistance (Zuber et al., 1999). As the second most abundant biological polymer after cellulose, lignin is important for stem stiffness and strength, and resistance against pests and pathogens (Boerjan et al., 2003; Bhuiyan et al., 2009; Zhang et al., 2014; Barros et al., 2015). Lignin is a phenylpropanoid-derived polymer produced by oxidative polymerization of the following three monolignol precursors in the plant cell wall: p-coumaryl alcohol (H unit), coniferyl alcohol (G unit), and sinapyl alcohol (S unit) (Vanholme et al., 2008). In addition to peroxidase, laccases are necessary for lignin polymerization through the oxidative polymerization of monolignols (Berthet et al., 2011; Zhao et al., 2013; Bryan et al., 2016).

A number of studies have reported that lignin biosynthesis can be altered by modifying the expression of a transcription factor that activates downstream target genes. These transcription factors include NACs, WRKYs, and MYBs (Mitsuda et al., 2007; Zhou et al., 2009; Wang et al., 2010). MicroRNAs (miRNAs) and other small RNAs are known to be important for gene regulation through post-transcriptional gene silencing, translational inhibition, or heterochromatin modification (Vaucheret et al., 2006). Recent studies found that miR397 and miR857 play important roles in lignin biosynthesis and secondary growth of vascular tissues in poplar and Arabidopsis, respectively (Lu et al., 2013; Wang et al., 2014; Zhao et al., 2015).

miR528 is a monocot-specific miRNA. In rice, miR528 targets an L-ascorbate oxidase (AO), a plastocyanin-like protein, a RING-H2 finger E3 ubiquitin ligase VirE2-interacting protein 2, and an F-box domain and leucine-rich repeat-containing protein DWARF3 (Wu et al., 2017). When rice is infected by viruses, miR528 preferentially associates with AGO18, resulting in enhanced AO activity, higher basal reactive oxygen species accumulation, and enhanced antiviral defence (Wu et al., 2017). Constitutive expression of rice miR528 enhances tolerance to salinity stress and N starvation in creeping bentgrass by repressing AAO (ascorbate acid oxidase) and CBP1 (copper ion binding protein 1) transcripts (Yuan et al., 2015). The functional significance of miR528 in maize, however, remains unclear because the predicted potential targets of ZmmiR528 are different from those in rice.

There is therefore a need to regulate lodging in valuable crops, such as corn. The present invention addresses this need.

### SUMMARY OF THE INVENTION

In the present application, we describe methods for improving lodging resistance in plants. Lodging usually happens fairly late in the growing season when the plants are large enough to be blown over (and when significant resources have been expended in growing the crop) and can render a crop completely unharvestable by mechanical means. Preventing lodging is therefore of huge economic importance. Moreover, unless the conditions for lodging occur in a growing season, it is difficult for breeders, farmers, crop testers or the like to determine whether a variety is resistant to lodging. As such, being able to determine whether an individual plant or variety will be resistant to lodging, should the conditions for lodging arise, is also very valuable.

We have found that lignin composition and content in maize are significantly affected by N supply and that ZmLACCASE3 (ZmLAC3) and ZmLACCASE5 (ZmLAC5) are the authentic targets of ZmmiR528. We show that that laccases, particularly LAC3 (ZmMZS) and LAC5 are involved in the regulation of lignin polymerization, resulting in changes in lignin content and thus causing the mechanical strength of the plants to change. We show that overexpression of these laccases promotes the increase of the lignin content in plants, thus improving lodging resistance. Conversely, inhibiting expression of laccases can reduce lignin content in plants, which increases the usefulness of the plant as a raw material for producing bioenergy and as forage for livestock. We also demonstrate that ZmmiR528, by negatively regulating the abundance of ZmLAC3 and ZmLAC5 mRNA, affects maize lignin biosynthesis and lodging resistance.

In one aspect of the invention, there is provided a method of altering resistance to lodging in a plant, the method comprising altering the expression or levels of at least one laccase gene and/or altering the expression or activity of miR528.

In a preferred embodiment, the method increases resistance to lodging in a plant by increasing the expression of at least one laccase gene and/or decreasing the expression or activity of miR528. More preferably, the laccase gene is selected from laccase 3 and laccase 5.

In one embodiment, the method comprises introducing and expressing a nucleic acid construct comprising at least one nucleic acid wherein the nucleic acid encodes a laccase 3 polypeptide as defined in SEQ ID NO: 1 or a functional variant or homologue thereof and/or a laccase 5 polypeptide as defined in SEQ ID NO: 4 or a functional variant or homologue thereof operably linked to a regulatory sequence.

In a preferred embodiment, the nucleic acid encoding laccase 3 comprises a sequence as defined in SEQ ID NO: 2 or 3 or a functional variant or homologue thereof and wherein the nucleic acid encoding laccase 5 comprises a sequence as defined in SEQ ID NO: 5 or 6 or a functional variant or homologue thereof.

In another embodiment, the method comprises introducing and expressing a nucleic acid construct comprising a nucleic acid sequence as defined in SEQ ID NO: 7 or 16 or a functional variant thereof operably linked to a regulatory sequence.

In a preferred embodiment, the regulatory sequence is a constitutive or strong promoter.

In one embodiment, the activity of the miR528 is decreased using at least one miR528 inhibitor. Preferably, the miR528 inhibitor is an RNA molecule comprising an RNA sequence as defined in SEQ ID NO: 8 or a functional variant thereof.

In an alternative embodiment, the method comprises introducing at least one mutation into at least one laccase gene, wherein the laccase polypeptide comprises at least one mutation in the miR528 binding site. Preferably, the laccase gene is selected from laccase 3 and laccase 5. More preferably, the laccase 3 gene encodes a polypeptide as defined SEQ ID NO: 1 and the laccase 5 gene encodes a polypeptide as defined in SEQ ID NO: 4. Even more preferably, the nucleic acid encoding laccase 3 comprises a sequence as defined in SEQ ID NO: 2 or 3 or a functional variant or homologue thereof and wherein the nucleic acid encoding laccase 5 comprises a sequence as defined in SEQ ID NO: 5 or 6 or a functional variant or homologue thereof.

In one embodiment, at least one mutation is introduced into at a least one position selected from positions 1 to 12 of SEQ ID NO: 3 or at least one position selected from positions 191 to 211 of SEQ ID NO: 2 or at least one position selected from positions 48 to 68 of SEQ ID NO: 6. More preferably, the mutation is introduced using targeted genome modification, preferably ZFNs, TALENs or CRISPR/Cas9. Alternatively, the mutation is introduced using mutagenesis, preferably TILLING or T-DNA insertion.

In an alternative embodiment, the method decreases resistance to lodging in a plant by decreasing the expression of at least one laccase gene and/or increasing the expression or activity of miR528.

In one embodiment, the laccase gene is selected from laccase 3 and laccase 5. More preferably, the laccase 3 gene encodes a polypeptide as defined SEQ ID NO: 1 and the laccase 5 gene encodes a polypeptide as defined in SEQ ID NO: 4. Even more preferably, the nucleic acid encoding laccase 3 comprises a sequence as defined in SEQ ID NO: 2 or 3 or a functional variant or homologue thereof and wherein the nucleic acid encoding laccase 5 comprises a sequence as defined in SEQ ID NO: 5 or 6 or a functional variant or homologue thereof.

In one embodiment, the method comprises introducing at least one mutation into at least one laccase gene and/or promoter, wherein the mutation decreases the expression of the laccase nucleic acid compared to a wild-type or control polypeptide. In a further embodiment, the method comprises introudicng at least one mutation into a miR528 and/or b gene or the miR528 promoter, preferably such that expression of miR528 (the precursor sequences or the mature sequence is reduced or abolished).

Preferably, the mutation is introduced using targeted genome modification, preferably ZFNs, TALENs or CRISPR/Cas9. Alternatively, the mutation is introduced using mutagenesis, preferably TILLING or T-DNA insertion.

In an alternative embodiment, the method comprises using RNA interference to reduce or abolish the expression of at least one laccase nucleic acid, preferably laccase 3 and/or 5 nucleic acid.

In one embodiment, the method comprises introducing and expressing a nucleic acid construct comprising at least one nucleic acid wherein the nucleic acid encodes a miR528 as defined in SEQ ID NO: 10 or a functional variant thereof operably linked to a regulatory sequence. Alternatively, the method comprises introducing an miR528 comprising SEQ ID NO: 9 (or 15) or a functional variant thereof.

Preferably, the plant is characterised by an increased lignin content compared to a control or wild-type plant.

In one embodiment, the expression or levels of at least one laccase gene and/or expression or activity of miR528 is altered compared to a wild-type or control plant. In another embodiment, root lodging resistance is altered compared to a control or wild-type plant.

In another aspect of the invention, there is provided a method of increasing at least one of yield, seed quality and stem strength, the method comprising increasing the expression of at least one laccase gene and/or decreasing the expression or activity of miR528.

In a further aspect of the invention, there is provided a method of altering lignin content in a plant, the method comprising altering the expression or levels of at least one laccase gene and/or altering the expression or activity of miR528.

In another aspect of the invention, there is provided a genetically altered plant, part thereof or plant cell, wherein said plant is characterised by altered expression or levels of at least one laccase gene and/or altered expression or activity of miR528. In an alternative embodiment, the plant is characterised by altered lignin content.

In one embodiment, the plant is characterised by an increased expression of at least one laccase gene and/or decreased expression or activity of miR528 compared to a wild-type or control plant.

Preferably, the plant expresses a nucleic acid construct comprising at least one nucleic acid wherein the nucleic acid encodes a laccase 3 polypeptide as defined in SEQ ID NO: 1 or a functional variant or homologue thereof and/or a nucleic acid encoding a laccase 5 polypeptide as defined in SEQ ID NO: 4 or a functional variant or homologue thereof operably linked to a regulatory sequence.

More preferably, the nucleic acid encoding laccase 3 comprises a sequence as defined in SEQ ID NO: 2 or 3 or a functional variant or homologue thereof and wherein the nucleic acid encoding laccase 5 comprises a sequence as defined in SEQ ID NO: 5 or 6 or a functional variant or homologue thereof.

In another embodiment, the plant expresses at least one miR528 inhibitor. In one embodiment, the plant expresses a nucleic acid construct comprising a nucleic acid sequence as defined in SEQ ID NO: 7 or 16 or a functional variant thereof operably linked to a regulatory sequence. In another embodiment, the plant expresses at least at least one miR528 inhibitor, wherein the miR528 inhibitor is an RNA molecule comprising an RNA sequence as defined in SEQ ID NO: 8 or a functional variant thereof.

In another embodiment, the plant comprises at least one mutation in at least one nucleic acid encoding a laccase nucleic acid, preferably wherein the laccase nucleic acid is selected from laccase 3 and 5, and wherein the mutation is in a miR528 binding site. Preferably, the mutation is introduced using targeted genome modification, preferably ZFNs, TALENs or CRISPR/Cas9. Alternatively, the mutation is introduced using mutagenesis, preferably TILLING or T-DNA insertion.

In one embodiment, the mutation introduced at a least one position selected from positions 1 to 12 of SEQ ID NO: 3 or at least one position selected from positions 48 to 68 of SEQ ID NO: 6.

In one embodiment, the plant is characterised by decreased expression of at least one laccase gene and/or increased expression or activity of miR528 compared to a wild-type or control plant.

In one embodiment, the plant expresses a nucleic acid construct comprising a nucleic acid sequence encoding at least one miR528. Preferably, the miR528 is as defined in SEQ ID NO: 10 or a functional variant thereof operably linked to a regulatory sequence or wherein the plant expresses a miR528 comprising SEQ ID NO: 9 or a functional variant thereof.

Preferably, the plant comprises at least one mutation in at least one nucleic acid encoding a laccase nucleic acid, preferably wherein the laccase nucleic acid is selected from laccase 3 and 5, and wherein the mutation decreases the expression of the laccase nucleic acid compared to a wild-type or control polypeptide. More preferably, the nucleic acid encodes a laccase 3 polypeptide as defined in SEQ ID NO: 1 or a functional variant or homologue thereof and/or a nucleic acid encoding a laccase 5 polypeptide as defined in SEQ ID NO: 4 or a functional variant or homologue thereof.

In a further embodiment, the plant comprises at least one mutation in a miR528 and/or b gene or the miR528 promoter, preferably such that expression of miR528 (the precursor sequences or the mature sequence is reduced or abolished).

In one embodiment, the mutation is introduced using targeted genome modification, preferably ZFNs, TALENs or CRISPR/Cas9 or wherein the mutation is introduced using mutagenesis, preferably TILLING or T-DNA insertion.

In another embodiment, the plant expresses an RNAi molecule that decreases the expression of at least one laccase 3 and/or 5 nucleic acid compared to a wild-type or control plant.

In a preferred embodiment, the plant is maize.

In another aspect of the invention, there is provided a method of producing a plant with altered resistance to lodging in a plant, the method comprising altering the expression or levels of at least one laccase gene and/or altering the expression or activity of miR528. In one embodiment the plant has an altered lignin content compared to a wild-type or control plant. More preferably, the plant has increased resistance to lodging in a plant, and the method comprises increasing the expression of at least one laccase gene and/or decreasing the expression or activity of miR528.

In one embodiment, the method comprises introducing and expressing a nucleic acid construct comprising at least one nucleic acid wherein the nucleic acid encodes a laccase 3 polypeptide as defined in SEQ ID NO: 1 or a functional variant or homologue thereof and/or a laccase 5 polypeptide as defined in SEQ ID NO: 4 or a functional variant or homologue thereof operably linked to a regulatory sequence. Preferably, the nucleic acid encoding laccase 3 comprises a sequence as defined in SEQ ID NO: 2 or 3 or a functional variant or homologue thereof and wherein the nucleic acid encoding laccase 5 comprises a sequence as defined in SEQ ID NO: 5 or 6 or a functional variant or homologue thereof.

In one embodiment, the method comprises introducing and expressing a nucleic acid construct comprising a nucleic acid sequence as defined in SEQ ID NO: 7 or 16 or a functional variant thereof operably linked to a regulatory sequence. Preferably, the regulatory sequence is a constitutive or strong promoter.

In an alternative embodiment, the activity of the miR528 is decreased using at least one miR528 inhibitor. Preferably, the miR528 inhibitor is an RNA molecule comprising an RNA sequence as defined in SEQ ID NO: 8 or a functional variant thereof.

In another alternative embodiment, the method comprises introducing at least one mutation into at least one laccase gene, wherein the laccase polypeptide comprises at least one mutation in the miR528 binding site, wherein preferably the laccase gene is selected from laccase 3 and laccase 5 and wherein the laccase 3 gene encodes a polypeptide as defined SEQ ID NO: 1 and wherein the laccase 5 gene encodes a polypeptide as defined in SEQ ID NO: 4. In a preferred embodiment, at least one mutation is introduced at at least one position selected from positions 1 to 12 of SEQ ID NO: 3 or at least one position selected from positions 48 to 68 of SEQ ID NO: 6.

In one embodiment, the mutation is introduced using introduced using targeted genome modification, preferably ZFNs, TALENs or CRISPR/Cas9 or wherein the mutation is introduced using mutagenesis, preferably TILLING or T-DNA insertion.

In another embodiment, the plant has decreased resistance to lodging and wherein the method comprises decreasing the expression of at least one laccase gene and/or increasing the expression or activity of miR528. Preferably, the laccase gene is selected from laccase 3 and/or laccase 5 and wherein the laccase 3 gene encodes a polypeptide as defined SEQ ID NO: 1 and wherein the laccase 5 gene encodes a polypeptide as defined in SEQ ID NO: 4.

In one embodiment, the method comprises introducing at least one mutation into at least one laccase gene and/or promoter, wherein the mutation decreases the expression of the laccase nucleic acid compared to a wild-type or control polypeptide.

Preferably, the mutation is introduced using targeted genome modification, preferably ZFNs, TALENs or CRISPR/Cas9 or wherein the method is introduced using mutagenesis, preferably TILLING or T-DNA insertion. In an alternative embodiment, the method comprises using RNA interference to reduce or abolish the expression of at least one laccase nucleic acid, preferably laccase 3 and/or 5 nucleic acid.

In another embodiment, the method comprises introducing and expressing a nucleic acid construct comprising at least one nucleic acid wherein the nucleic acid encodes a miR528 as defined in SEQ ID NO: 10 or a functional variant thereof operably linked to a regulatory sequence. Alternatively, the method comprises introducing an miR528 comprising SEQ ID NO: 9 or 15 or a functional variant thereof.

In one embodiment, the method further comprises measuring an alteration in lodging, preferably compared to a control or wild-type plant. More preferably, the method further comprises regenerating a plant and screening for an alteration in lodging.

In a preferred embodiment, the plant is maize.

In another aspect of the invention, there is provided a plant obtained or obtainable by any of the methods described above.

In a further aspect there is provided a nucleic acid construct comprising a nucleic acid sequence encoding a miR528 inhibitor as defined in SEQ ID NO: 7 or 16 or a functional variant thereof. Also provided is a vector comprising the nucleic acid construct described herein.

In another aspect, there is provided a miR528 inhibitor comprising an RNA molecule with an RNA sequence as defined in SEQ ID NO: 8 or a functional variant thereof.

In another aspect of the invention, there is provided, a host cell comprising the nucleic acid construct as described herein, the vector as described herein or the miR528 inhibitor as described herein. Preferably, the host cell is a bacterial or plant cell.

In another aspect, there is provided a transgenic plant expressing the nucleic acid construct, vector or miR528 inhibitor as described herein. Most preferably the plant is maize.

In another aspect there is provided the use of the nucleic acid construct, vector or miR528 inhibitor as described herein to increase lodging resistance in a plant compared to a control or wild-type plant. There is also provided the use of the nucleic acid construct, vector or miR528 inhibitor as described herein to regulate lignin synthesis, regulate lignin content and/or promote lignin synthesis in a plant compared to a control or wild-type plant. Preferably, said nucleic acid construct, vector or miR528 inhibitor increases lignin synthesis and/or increases lignin content in a plant compared to a control or wild-type plant. More preferably, lignin synthesis and/or lignin content is increased in plant stems and/or roots. Most preferably the plant is maize.

In a further aspect of the invention, there is provided a nucleic acid construct comprising a nucleic acid sequence encoding at least one DNA-binding domain that can bind to at least one miR528 gene. Alternatively, there is provided a nucleic acid construct that comprises a nucleic acid sequence encoding at least one DNA-binding domain that can bind to a LAC3 or LAC5 gene and inhibit or prevent the binding of miR528 at the miR528 binding site. More preferably, laccase activity is unaffected.

In one embodiment, the nucleic acid sequence encodes at least one protospacer element, wherein the sequence of the protospacer element is selected from SEQ ID NO: 34, 37, 41, 44, or a variant thereof. In another embodiment, the nucleic acid sequence encodes at least one protospacer element, wherein the sequence of the protospacer element is selected from SEQ ID NO: 52, 55, 58 or 61 or a variant thereof.

In a further preferred embodiment, the construct further comprises a nucleic acid sequence encoding a CRISPR RNA (crRNA) sequence, wherein said crRNA sequence comprises the protospacer element sequence and additional nucleotides.

More preferably, the construct further comprises a nucleic acid sequence encoding a transactivating RNA (tracrRNA), wherein preferably the tracrRNA is defined in SEQ ID NO: 31 or a functional variant thereof.

In a further embodiment, the construct encodes at least one single-guide RNA (sgRNA), wherein said sgRNA comprises the tracrRNA sequence and the crRNA sequence. In one embodiment, the sgRNA comprises or consists of a sequence selected from SEQ ID NO: 35, 38, 42, 45, or a functional variant thereof. In a further embodiment, the sgRNA comprises or consists of a sequence selected from SEQ ID NO: 53, 56, 59 or 62 or a functional variant thereof.

In one embodiment, the protospacer element or sgRNA is operably linked to a regulatory sequence, where preferably the regulatory sequence is a promoter, more preferably a constitutive promoter.

In a further embodiment, the nucleic acid construct further comprises a nucleic acid sequence encoding a CRISPR enzyme. Preferably, the CRISPR enzyme is a Cas or Cpf1 protein. More preferably, the Cas protein is Cas9 or a functional variant thereof.

In an alternative embodiment, the nucleic acid construct encodes a TAL effector. Preferably, the nucleic acid construct further comprises a sequence encoding an endonuclease or DNA-cleavage domain thereof. More preferably, the endonuclease is Fokl.

In another aspect of the invention there is provided a single guide (sg) RNA molecule wherein said sgRNA comprises a crRNA sequence and a tracrRNA sequence. In one embodiment, the crRNA sequence can bind to at least one sequence selected from SEQ ID NO: 33, 36, 40, 43 or a variant thereof. In another embodiment, the crRNA sequence can bind to at least one sequence selected from SEQ ID NO: 51, 54, 57 or 60 or a variant thereof.

In another aspect, there is provided an isolated plant cell transfected with at least one nucleic acid construct described herein or at least one sgRNA described herein. In one embodiment, the isolated plant cell is transfected with at least one nucleic acid construct comprising a protospacer element or sgRNA as described herein and a second nucleic acid construct, wherein said second nucleic acid construct comprises a nucleic acid sequence encoding a Cas protein, preferably a Cas9 protein or a functional variant thereof. In this embodiment, the second nucleic acid construct is transfected before, after or concurrently with the sgRNA construct.

In another aspect of the invention there is provided a genetically modified plant, wherein said plant comprises the transfected cell described herein. In one embodiment, the nucleic acid encoding the sgRNA and/or the nucleic acid encoding a Cas protein is integrated in a stable form.

In another aspect of the invention there is provided a method of increasing resistance to lodging in a plant, the method comprising introducing and expressing in a plant the nucleic acid construct described herein, or the sgRNA described herein, wherein preferably said increase is relative to a control or wild-type plant.

In a further aspect, there is provided a plant obtained or obtainable by any of the methods described herein.

In another aspect, there is provided the use of a nucleic acid construct described herein or the sgRNA described herein to increase resistance to lodging in a plant. Preferably, the nucleic acid construct or sgRNA decreases the expression and/or activity of miRNA528 in a plant.

In a further aspect of the invention, there is provided a method for obtaining the genetically modified plant described herein, the method comprising:
a) selecting a part of the plant;
b) transfecting at least one cell of the part of the plant of paragraph (a) with the nucleic acid construct described herein or the sgRNA molecule described herein;
c) regenerating at least one plant derived from the transfected cell or cells;
d) selecting one or more plants obtained according to paragraph (c) that show reduced expression of at least one miRNA528 nucleic acid in said plant.

In another aspect, there is provided a method for identifying and/or selecting a plant that will have increased lodging resistance, preferably compared to a wild-type or control plant, the method comprising detecting in the plant or plant germplasm at least one polymorphism or mutation in a laccase 3 gene and/or promoter, laccase 5 gene and/or promoter or miR528a and/or b gene and/or promoter wherein said polymorphism or mutation results in an increased expression of laccase 3 and/or 5 and/or reduced expression/activity of miR528 compared to a plant without said mutation; and selecting said plant or progeny thereof.

In another aspect of the invention, there is provided a protein, which is

(a) a protein comprising an amino acid sequence as shown in SEQ ID NO:1; or
(b) a plant lignin synthesis-related protein having an amino acid sequence derived from SEQ ID NO: 1 by the substitution and/or deletion and/or addition of one or more amino acid residues in, from or to the amino acid sequence as shown in SEQ ID NO: 1.

There is also provided a coding gene of the protein described above, wherein preferably the coding gene is a DNA molecule selected from any one of:

(1) a DNA molecule having a coding region as shown in SEQ ID NO: 3;
(2) a DNA molecule that hybridizes with the DNA sequence of (1) under stringent conditions and encodes a plant lignin synthesis-related protein; and
(3) a DNA molecule that is at least 90% homogeneous to the DNA sequence of (1) and encodes a plant lignin synthesis-related protein.

There is further provided a recombinant expression vector, an expression cassette, a transgenic cell line or a recombinant strain, comprising the gene described above.

In another aspect of the invention, there is provided the use of the protein described herein in at least one of

(d1) the regulation of lignin synthesis in plants;
(d2) the regulation of lignin synthesis in plant stems;
(d3) the regulation of the lignin content in plants;
(d4) the regulation of the lignin content in plant stems;
(d5) the promotion of lignin synthesis in plants;
(d6) the promotion of lignin synthesis in plant stems;
(d7) the promotion of the increase in lignin content in plants; and
(d8) the promotion of the increase in lignin content in plant stems.

There is also provided a method for producing a transgenic plant, comprising the steps of: introducing the gene described above into a starting plant, to obtain a transgenic plant, wherein compared with the starting plant, the transgenic plant has a phenotype of (f1) increased lignin content; or (f2) increased lignin content in the stems.

There is also provided a method for producing a transgenic plant, comprising the steps of: inhibiting the expression of the gene described above in a starting plant to obtain a transgenic plant, wherein compared with the starting plant, the transgenic plant has at least a phenotype of (g1) reduced lignin content; or (g2) reduced lignin content in the stems.

There is also provided a method of plant breeding, by increasing the content and/or activity of the protein described herein in a target plant, so as to increase the lignin content in the target plant or increase the lignin content in the stems of the target plant.

There is also provided a method of plant breeding, by reducing the content and/or activity of the protein described herein in a target plant, so as to reduce the lignin content in the target plant or reduce the lignin content in the stems of the target plant.

There is also provided the use of the protein, gene, recombinant expression vector or method described above in plant breeding.

In another aspect of the invention, there is provided a method for breeding a transgenic plant with different lignin content. In one embodiment, there is provided a method for provided a method of breeding a plant with reduced lignin content, comprising the following steps: introducing a specific DNA molecule I into a starting plant to obtain a transgenic plant with a lower lignin content than the starting plant, wherein the specific DNA molecule I is a DNA molecule A or a DNA molecule B, wherein the DNA molecule A encodes a miRNA having a sequence as shown in SEQ ID NO: 15 or 9, and the DNA molecule B encodes a precursor RNA of the miRNA having a sequence as shown in SEQ ID NO: 15 or 9.

There is also provided the method described above wherein the “precursor RNA of the miRNA having a sequence as shown in SEQ ID NO: 15 or 9” is a RNA having a sequence as shown in SEQ ID NO: 14.

In one aspect, the specific DNA molecule I may be specifically a DNA molecule having a sequence as shown in SEQ ID NO: 13.

In another embodiment, the specific DNA molecule I may be specifically introduced into the starting plant by a recombinant plasmid I. The recombinant plasmid I may be specifically a recombinant plasmid obtained by inserting the specific DNA molecule I into the BamHI cleavage site of the pCUB vector.

In another embodiment, the transgenic plant obtained from the method described above has reduced lignin content and reduced puncture strength of the stem, and can be used as a raw material for producing bioenergy.

There is also provided a method for breeding a transgenic plant with increased lignin content, comprising the following steps: introducing a specific DNA molecule II into a starting plant to obtain a transgenic plant with a higher lignin content than the starting plant, wherein the specific DNA molecule II is a DNA molecule inhibiting the expression of a miRNA having a sequence as shown in SEQ ID NO: 15 or 9. In one embodiment, the specific DNA molecule II may be specifically a DNA molecule having a sequence as shown in SEQ ID NO: 7.

In another embodiment, the specific DNA molecule II may be specifically introduced into the starting plant by a recombinant plasmid II. The recombinant plasmid II may be specifically a recombinant plasmid obtained by inserting the specific DNA molecule II into the BamHI cleavage site of the pCUB vector.

In another embodiment, the transgenic plant obtained from the method described above has increased lignin content, increased stem puncture strength and improved lodging resistance.

In one embodiment, any of the starting plants mentioned plants above may be a monocotyledonous plant. The monocotyledonous plant may be a gramineous plant, and in particular corn, such as Maize Variety 31.

In another aspect of the invention, there is provided a miRNA having a sequence as shown in SEQ ID NO: 15 or 9. There is also provided a precursor RNA of the miRNA having a sequence as shown in SEQ ID NO: 15 or 9. Preferably, the precursor RNA is specifically a RNA having a sequence shown in SEQ ID NO: 14.

There is also provided an RNA having a sequence as shown in SEQ ID NO: 14.

There is also provided a gene encoding a miRNA having a sequence as shown in SEQ ID NO: 15 or 9 or an RNA having a sequence as shown in SEQ ID NO: 14. Preferably, the gene is specifically a DNA molecule having a sequence as shown in SEQ ID NO: 13.

There is further provided a recombinant vector comprising the gene as described above.

There is also provided the use of the miRNA, RNA or gene described above in the breeding of plants with reduced lignin content.

In another aspect of the invention, there is provided use of a substance having inhibition on the expression of the miRNA or the RNA described above in the breeding of plants with increased lignin content.

There is also provided a recombinant vector comprising the gene of the present invention. The recombinant vector is specifically a recombinant plasmid obtained by inserting the gene into the BamHI cleavage site of the pCUB vector. There is also provided a recombinant plasmid comprising a DNA molecule having a sequence as shown in SEQ ID NO: 5.

In another aspect, there is provided use of a compound having inhibition on the expression of the miRNA having a sequence as shown in SEQ ID NO: 15 or 9 or the RNA having a sequence as shown in SEQ ID NO: 14 in the breeding of plants with increased lignin content. The compound having inhibition on the expression of the miRNA having a sequence as shown in SEQ ID NO: 15 or 9 or the RNA having a sequence as shown in SEQ ID NO: 14 is specifically an interference vector. The interference vector is a recombinant plasmid containing a DNA molecule having a sequence as shown in SEQ ID NO: 7. The interference vector is specifically a recombinant plasmid obtained by inserting the DNA molecule having a sequence as shown in SEQ ID NO: 7 into the BamHI cleavage site of the pCUB vector.

There is also provided a recombinant plasmid (interference vector), which is a recombinant plasmid containing a DNA molecule having a sequence as shown in SEQ ID NO: 7. The interference vector is specifically a recombinant plasmid obtained by inserting the DNA molecule having a sequence as shown in SEQ ID NO: 7 into the BamHI cleavage site of the pCUB vector.

Any of the above plants may be a monocotyledonous plant. The monocotyledonous plant may be a gramineous plant, and in particular corn, such as Maize Variety 31.

### DESCRIPTION OF THE FIGURES

The invention is further described in the following non-limiting figures:

FIG. 1 shows the relative expression level of a ZmMZS gene in Example 1.

FIG. 2 shows microphotographs after histological staining in Example 1.

FIG. 3 shows the lignin content determined in Example 1.

FIG. 4 shows microphotographs after histological staining of the root in Example 2.

FIG. 5 shows microphotographs after histological staining of the first internode in Example 2.

FIG. 6 shows the lignin content determined in Example 2.

FIG. 7 shows the miRNA expression level identified.

FIG. 8 shows microphotographs after histological staining of the middle root portion.

FIG. 9 shows microphotographs after histological staining of the first internode.

FIG. 10 shows the lignin content determined.

FIG. 11 shows the puncture strength of the stem determined.

FIG. 12 shows a photograph of plants in the tasseling stage in the pot experiment.

FIG. 13 shows the result of Northern Blot analysis.

FIG. 14 shows regulation of ZmmiR528 and ZmLACs expression by N supply. Regulation of (A) ZmmiR528 and (B and C) its targets by N supply. Plants were grown hydroponically in a nutrient solution containing 0.02, 2, or 4 mM Ca(NO₃)₂ for the indicated time before RNA was isolated from leaves and roots. The expression levels of ZmmiR528 and its targets were normalized to that of ZmUBQ1. NL, NS, and ND indicate N luxury, N sufficiency, and N deficiency, respectively. Values are means±SE of four biological replicates. Means with the same letter are not significantly different at p<0.01 according to the least significant difference (LSD) test.

FIG. 15 shows ZmLAC3 and ZmLAC5 are regulated by ZmmiR528. (A) Co-expression of the constructs containing ZmMIR528b and ZmLAC3 or ZmLAC5 in N. benthamiana leaves. Expression levels determined by real-time RT-PCR were normalized to the expression levels of tobacco 18S rRNA. Values are the means±SE of three biological replicates. Means with the same letter are not significantly different at p<0.01 according to the LSD test. (B) ZmLAC3 and ZmLAC5 mRNA cleavage sites determined by 5′ RACE. Numbers indicate the frequency of cleavage at each site.

FIG. 16 shows expression patterns of ZmmiR528 and ZmLAC5 determined by in situ hybridization analysis. Accumulation of (A) ZmmiR528 and (B) ZmLAC5 transcripts in the roots, stems, and shoots of hydroponically grown maize. Corresponding sense probes were used as negative controls. Representative plants were photographed. Scale bars in (A) and (B) represent 100 μm and 50 μm, respectively.

FIG. 17 shows maize lodging resistance is affected by ZmmiR528 abundance. (A) Soil-grown ZmmiR528-overexpressing transgenic maize was more sensitive to lodging under N-luxury conditions than wild-type or transgenic ZmmiR528 knock-down lines. Representative plants were photographed. WT, wild-type. (B) The effects of ZmmiR528 abundance on the rind penetrometer resistance of stems of soil-grown maize. Ten plants of each genotype were measured. (C and D) The effects of ZmmiR528 abundance on AcBr lignin (C), cellulose, and hemicellulose (D) contents in the stems of soil-grown maize. DW represents dry weight. Values are the means±SE of four biological replicates. Means with the same letter are not significantly different at p<0.01 according to the LSD test. (E) ZmmiR528 levels in WT, TM, and OE transgenic maize as indicated by small RNA northern blots. Five micrograms of small RNA from each sample was loaded per lane. U6 was shown as loading controls. Numbers below each lane indicate the relative expression ratio. (F) The effects of ZmmiR528 abundance on AcBr lignin content, Values are the means±standard error of four biological replicates. DW represents dry weight. Means with the same letter are not significantly different at P<0.01 according to the LSD test. (G) Detection of corresponding ZmLAC3 and ZmLAC5 gene transcripts in WT, TM, and OE transgenic maize as indicated by real-time RT-PCR. Quantifications were normalized to the expression of ZmUBQ1. Values are means±standard error of three biological replicates. Means with the same letter are not significantly different at P<0.01 according to the LSD test.

FIG. 18 shows ZmLAC3 overexpression increases lignin content in soil-grown maize. (A) Phloroglucinol staining of the stems of ZmLAC3-overexpressing transgenic maize. Representative plants were photographed. Scale bars represent 75 μm. (B-E) AcBr lignin content (B), rind penetrometer resistance (C), cellulose content (D), and hemicellulose content (E) of ZmLAC3-overexpressing transgenic maize. DW represents dry weight. Values are the means±SE of four biological replicates. Means with the same letter are not significantly different at p<0.01 according to the LSD test.

FIG. 19 shows (A) the mRNA levels of ZmLAC3 in ZmLAC3OE transgenic maize. Real-time RT-PCR quantifications were normalized to the expression of ZmUBQ1. Values are means±standard error of four biological replicates. Means with the same letter are not significantly different at P<0.01 according to the LSD test. Also shows (B) the ZmPALs transcripts in ZmLAC3OE transgenic maize. The expression levels were normalized to that of ZmUBQ1. Values are means±standard error of three biological replicates. Means with the same letter are not significantly different at P<0.01 according to the LSD test.

FIG. 20 shows the effects of N supply on ZmPAL transcript levels in WT, TM, and OE transgenic maize. The effects of ZmmiR528 abundance and N supply on ZmPAL transcript levels. The expression levels were normalized to those of ZmUBQ1. Values are means±SE of three biological replicates. Means with the same letter are not significantly different at p<0.01 according to the LSD test. NL, NS, and ND indicate N luxury, N sufficiency, and N deficiency, respectively.

FIG. 21 shows a proposed model for the role of ZmmiR528 in maize lodging resistance under N-luxury conditions. The increased levels of miR528, and decreased abundance of ZmLACs and ZmPALs could explain the reduced lodging resistance of maize under N-luxury conditions. Arrows indicate positive regulation and blunt-ended bars indicate inhibition.

FIG. 22 shows a schematic diagram of sgRNAs and hSpCas9 used in Example 7.

FIG. 23 shows the effect on lodging resistance of crossing a miR527 knockdown transgenic plant with a lodging-prone plant.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry and recombinant DNA technology, bioinformatics which are within the skill of the art. Such techniques are explained fully in the literature.

As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences.

The terms “polypeptide” and “protein” are used interchangeably herein and refer to amino acids in a polymeric form of any length, linked together by peptide bonds.

The term “miR528” refers to a micro(mi) RNA molecule. The RNA sequence of mature miR528 is shown in SEQ ID NO: 9 and 15. The DNA sequence encoding the mature miR528 is shown in SEQ ID NO: 10. In one example, miR528 is maize miR528, also referred to herein as “ZmmiR528”. In maize there are two members of miR528—miR528a and miR528b, each encoded by a different locus. Each locus produces a miR528 with a different precursor sequence (shown as SEQ ID NO: 32 and 39 corresponding to miR528a and b respectively) although the mature sequence produced is identical. The term “precursor” refers to a precursor RNA or pre-miRNA which is processed within host cells to generate a short, partially double stranded RNA in which one strand is the mature miRNA.

The aspects of the invention involve recombination DNA technology and exclude embodiments that are solely based on generating plants by traditional breeding methods.

In one aspect of the invention there is provided a method of altering resistance to lodging in a plant, the method comprising altering the expression or levels of at least one laccase gene and/or altering the expression or activity of miR528.

In one embodiment “altering” may mean increasing resistance to lodging. In an alternative embodiment, “altering” may mean decreasing resistance to lodging. In one embodiment, the increase or decrease may be up to or be at least 1%, 2%, 3%, 4%, 5%, 6%, 7%, 8%, 9%, 10%, 20%, 30%, 40%, 50%, 60%, 70%, 80%, 90 or 95% or more compared to a control plant. In one example the level of increase may be between 12 and 20%.

In one embodiment, resistance to lodging is altered under high nitrogen or nitrogen-rich conditions. In one example, high N may be considered above 300 kg urea/ha. In an alternative embodiment, resistance to lodging is altered under normal (e.g. 240-300 kg urea/ha) or low nitrogen conditions (180 kg urea/ha or lower, preferably between 180 and 120 kg urea/ha).

As used herein, the term “resistance to lodging” or “lodging resistance” can also be referred to as “harvestability” and may refer to the bending or breakage of the plant stem, or the tilting over of the plant. Alternatively, an increase in resistance to lodging can be considered equivalent to a decrease in lodging and a decrease in resistance to lodging can be considered as equivalent to an increase in lodging.

In one embodiment, lodging is increased or decreased in the stem of a plant and/or the roots of a plant.

In one example, lodging severity can be scored visually for a plot where stalk lodging is visible by the breakage of the stalk at or below the ear, and where root lodging is visible by maize stems tilting at an angle that exceeds 30° C.

In another example, lodging resistance can be determined from a measure of stalk strength. One measure of stalk strength is rind penetration or penetrometer resistance or RPR (which is a measure of the force needed to pierce a stalk rind with a spike or needle). A number of studies have demonstrated that rind penetration resistance negatively correlates with stalk lodging in the field. In one embodiment, RPR may be increased or decreased by up to or at least 1%, 2%, 3%, 4%, 5%, 6%, 7%, 8%, 9%, 10%, 20%, 30%, 40%, 50%, 60%, 70%, 80%, 90 or 95% or more compared to a control plant. In one example the level of increase may be between 12 and 20%.

In a further example, lodging resistance can be determined by an increase in lignin content in the plant, preferably in the stem and/or root of the plant. As the second most abundant biological polymer after cellulose, lignin is important for stem stiffness and strength, and resistance against pests and pathogens (Boerjan et al., 2003; Bhuiyan et al., 2009; Zhang et al., 2014; Barros et al., 2015). Lignin is a phenylpropanoid-derived polymer produced by oxidative polymerization of the following three monolignol precursors in the plant cell wall: p-coumaryl alcohol (H unit), coniferyl alcohol (G unit), and sinapyl alcohol (S unit) (Vanholme et al., 2008). In one example, to determine total lignin polymer content, plant material is subject to hydrolysis by acetyl bromide (AcBr) and lignin content analysed. This is known as the AcBr method and is described by Fukushima and Hatfield (2004). In one embodiment, lignin content may be increased or decreased by up to or at least 1%, 2%, 3%, 4%, 5%, 6%, 7%, 8%, 9%, 10%, 20%, 30%, 40%, 50%, 60%, 70%, 80%, 90 or 95% or more compared to a control plant. In one example the level of increase may be between 12 and 20%.

In a further example, lodging resistance can be determined from an increase in cellulose and/or hemicellulose content in the plant, preferably the stem and/or root of the plant. In one example, cellulose and/or hemicellulose content can be determined using modified NREL procedures (as described in Sluiter et al., 2008). In one embodiment, cellulose and/or hemicellulose content may be increased or decreased by up to or at least 1%, 2%, 3%, 4%, 5%, 6%, 7%, 8%, 9%, 10%, 20%, 30%, 40%, 50%, 60%, 70%, 80%, 90 or 95% or more compared to a control plant. In one example the level of increase may be between 12 and 20%.

Accordingly, in one example, lodging resistance can be determined from a measurement of any one or a combination of the following: a visual score of lodging severity, rind penetrometer resistance, lignin content and/or cellulose/hemicellulose content. Other parameters to measure lodging resistance would be known to the skilled person.

In one embodiment, the method increases resistance to lodging in a plant by increasing the level or expression of at least one laccase gene.

In another aspect of the invention, there is provided a method of increasing yield, and/or stem strength, particularly under lodging conditions or when the plant has been exposed to conditions that will result in lodging in a wild-type or control plant, the method comprising increasing the expression of at least one laccase gene and/or decreasing the expression or activity of miR528. In one example, lodging conditions are any environmental conditions, such as high winds, rain, overpopulation, storm damage etc. that would cause lodging in the wild-type or control plant.

The term “yield” in general means a measurable produce of economic value, typically related to a specified crop, to an area, and to a period of time. Individual plant parts directly contribute to yield based on their number, size and/or weight. The actual yield is the yield per square meter for a crop per year, which is determined by dividing total production per year (includes both harvested and appraised production) by planted square metres.

Yield is increased relative to a control or wild-type plant. For example, the yield is increased by 2%, 3%, 4%, 5%, 6%, 7%, 8%, 9%, 10%, 11%, 12%, 13%, 14%, 15%, 16%, 17%, 18%, 19% or 20%, 25%, 30%, 35%, 40%, 45% or 50% compared to a control or wild-type plant.

In a further aspect of the invention, there is provided a method of altering lignin content in a plant, the method comprising altering the expression or levels of at least one laccase gene and/or altering the expression or activity of miR528. In a preferred embodiment, the method comprises increasing the lignin content in a plant, preferably in the stem or root of a plant, the method comprising increasing the expression of at least one laccase gene and/or decreasing the expression or activity of miR528. In one embodiment, lignin content may be increased or decreased by up to or at least 1%, 2%, 3%, 4%, 5%, 6%, 7%, 8%, 9%, 10%, 20%, 30%, 40%, 50%, 60%, 70%, 80%, 90 or 95% or more compared to a control plant.

As used herein “increasing the expression” means an increase in the nucleotide levels and “increasing the levels” as used herein means an increase in the protein levels of at least one laccase. In one embodiment, the expression or levels or activity of at least one laccase are increased by up to or more than 5%, 10%, 20%, 30%, 40%, 50%, 60%, 70%, 80% or 90% when compared to the level in a wild-type or control plant. Methods for determining laccase nucleotide expression or protein levels would be well known to the skilled person. In particular increases can be measured by any standard technique known to the skilled person. For example, an increase in the expression and/or protein levels of a laccase may comprise a measure of protein and/or nucleic acid levels and can be measured by any technique known to the skilled person, such as, but not limited to, any form of gel electrophoresis or chromatography (e.g. HPLC).

Laccases are copper-containing oxidase enzymes. As used herein, laccases may also be referred to as plant lignin synthesis-related proteins. In a preferred embodiment, the laccase is selected from laccse 3 (or LAC3) and laccse 5 (or LAC5). In one embodiment, the plant is maize and the laccase may be referred to as ZmLACCASE 3 (ZmLAC3) or ZmLACCASE 5 (ZmLAC5). Alternatively ZmLAC3 may be referred to herein as ZmMNS or ZmMZS.

In one aspect of the invention there is provided a laccase protein, wherein the protein is
(a) a protein comprising an amino acid sequence as shown in SEQ ID NO:1 or 4; or
(b) a plant lignin synthesis-related protein having an amino acid sequence derived from SEQ ID NO: 1 by the substitution and/or deletion and/or addition of one or more amino acid residues in, from or to the amino acid sequence as shown in SEQ ID NO: 1.

For ease of the purification and detection of the protein in (b), the N or C terminus of the protein comprising the amino acid sequence as shown in SEQ ID NO: 1 can be attached with a tag as shown in Table 1.

TABLE 1
Tag sequences:

| Tag | Residue | Sequence |
|---|---|---|
| Poly-Arg | 5-6 | RRRRR (SEQ ID |
| | (generally 5) | NO: 67) |
| Poly-His | 2-10 | HHHHHH (SEQ ID |
| | (generally 6) | NO: 68) |
| FLAG | 8 | DYKDDDDK (SEQ |
| | | ID NO: 69) |
| Strep-tag II | 8 | WSHPQFEK |
| | | (SEQ ID NO: 70) |
| c-myc | 10 | EQKLISEEDL |
| | | (SEQ ID NO: 71) |

The protein in (b) may be artificially synthesized, or be obtained by synthesizing a coding gene thereof followed by biological expression. The coding gene of the protein in (b) may be obtained by deleting the codon(s) of one or more amino acid residues and/or undergoing missense mutation of one or more base pairs in a DNA sequence as shown in SEQ ID NO: 3, and/or attaching a tag as shown in Table 1 above to the 5′ and/or 3′ terminus of a coding sequence thereof.

In another aspect of the invention, there is also provided an isolated laccase nucleic acid or DNA molecule. In one embodiment, the DNA molecule is selected from any one of
(1) a DNA molecule having a coding region as shown in SEQ ID NO: 3 or 6;
(2) a DNA molecule having the genomic sequence shown in SEQ ID NO: 2 or 5;
(3) a DNA molecule that hybridizes with the DNA sequence of (1) or (2) under stringent conditions and encodes a plant lignin synthesis-related or laccase protein; and
(4) a DNA molecule that is at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% homogeneous (or “identical”) to the DNA sequence of (1), (2) or (3) and encodes a plant lignin synthesis-related protein or laccase.

Preferably, the stringent conditions may comprise hybridization in 0.1×SSPE (or 0.1×SSC) and 0.1% SDS solution in a DNA or RNA hybridization experiment at 65° C. and washing.

In another aspect of the invention, there is provided a recombinant expression vector (also referred to herein as a “nucleic acid construct”), an expression cassette, a transgenic cell line or recombinant strain comprising a protein, nucleic acid or DNA molecule described herein, preferably ZmMZS.

The recombinant expression vector comprising a laccase nucleic acid as described herein, preferably LAC3 (preferably ZmMZS) or LAC5 may be constructed by using an existing expression vector. The expression vector comprises binary Agrobacterium tumefaciens vector and vectors for microprojectile bombardment. When a recombinant expression vector is constructed with the laccase gene, any of an enhanced, constitutive, tissue-specific, or inducible promoter may be linked before the transcription initiation nucleotide, which may be used alone or in combination with other plant promoters. Moreover, when a recombinant expression vector is constructed with a laccase gene, an enhancer may be included, including a translational enhancer or a transcriptional enhancer. These enhancer regions may be the ATG initiation codon or an initiation codon of an adjacent region, which however needs to be co-framed with the coding sequence, to ensure the proper translation of the whole sequence. The translation control signal and the initiation codon are widely available, and may be natural, or synthesized. The translation initiation region may be from a transcription initiation region or a structural gene. To facilitate the identification and screening of transgenic plants or transgenic microorganisms, the expression vectors used may be processed, for example, by adding a gene expressing enzymes or luminescent compounds that produce colour changes in plants or microorganisms, resistant antibiotic markers, or chemical resistant marker genes. Considering the safety of the transgene, the plants or microorganisms may be directly transformed by phenotypic selection without adding a selective marker gene.

Specifically, the recombinant expression vector may be a recombinant plasmid pCUB-ZmMZS obtained by inserting a DNA molecule as shown in any of SEQ ID NO: 2, 3 5 or 6 into the BamHI cleavage site of the pCUB vector.

In one embodiment, the method comprises introducing and expressing a nucleic acid construct comprising at least one nucleic acid wherein the nucleic acid encodes a laccase 3 and/or laccase 5 polypeptide as described above, operably linked to a regulatory sequence. Use of the nucleic acid construct described above leads to the expression, preferably overexpression of laccase 3 and/or laccase 5 in the plant where the nucleic acid is introduced.

Preferably, the laccase 3 polypeptide is as defined in SEQ ID NO: 1 or a functional variant or homologue thereof. Accordingly, in one embodiment, the laccase 3 nucleic acid encodes a polypeptide as defined in SEQ ID NO: 1 or a variant thereof. More preferably the laccase 3 nucleic acid comprises or consists of SEQ ID NO: 2 or 3 or a functional variant thereof.

Preferably, the laccase 5 polypeptide is as defined in SEQ ID NO: 4 or a functional variant or homologue thereof. Accordingly, in one embodiment, the laccase 5 nucleic acid encodes a polypeptide as defined in SEQ ID NO: 4 or a variant thereof. More preferably the laccase 5 nucleic acid comprises or consists of SEQ ID NO: 5 or 6 or a functional variant or homologue thereof.

The term “variant” or “functional variant” as used herein with reference to any of SEQ ID NOs: 1 to 47 refers to a variant gene sequence or part of the gene sequence which retains the biological function of the full non-variant sequence. A functional variant also comprises a variant of the gene of interest, which has sequence alterations that do not affect function, for example in non-conserved residues. Also encompassed is a variant that is substantially identical, i.e. has only some sequence variations, for example in non-conserved residues, compared to the wild type sequences as shown herein and is biologically active. Alterations in a nucleic acid sequence that result in the production of a different amino acid at a given site that does not affect the functional properties of the encoded polypeptide are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

As used in any aspect of the invention described herein a “variant” or a “functional variant” has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid or amino acid sequence.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognised that residue positions that are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Non-limiting examples of algorithms that are suitable for determining percent sequence identity and sequence similarity are the BLAST and BLAST 2.0 algorithms.

In a further embodiment, a variant as used herein can comprise a nucleic acid sequence encoding a laccase polypeptide as defined herein that is capable of hybridising under stringent conditions as defined herein to any one of SEQ ID NO: 2, 3, 5 or 6.

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than about 24 hours, usually about 4 to 12 hours. Stringent conditions may also be achieved with the addition of destabilizing agents such as formamide. In one example, the stringent conditions may comprise hybridisation in 0.1×SPPE (or 0.1×SSC) and 0.1% SDS solution in a DNA or RNA hybridisation experiment at 65° C. and washing.

According to all aspects of the invention, including the method above and including the plants, methods and uses as described below, the term “regulatory sequence” is used interchangeably herein with “promoter” and all terms are to be taken in a broad context to refer to regulatory nucleic acid sequences capable of effecting expression of the sequences to which they are ligated. The term “regulatory sequence” also encompasses a synthetic fusion molecule or derivative that confers, activates or enhances expression of a nucleic acid molecule in a cell, tissue or organ.

In one embodiment, the promoter may be a constitutive or a strong promoter.

A “constitutive promoter” refers to a promoter that is transcriptionally active during most, but not necessarily all, phases of growth and development and under most environmental conditions, in at least one cell, tissue or organ. Examples of constitutive promoters include the cauliflower mosaic virus promoter (CaMV35S or 19S), rice actin promoter, maize ubiquitin promoter, rubisco small subunit, maize or alfalfa H3 histone, OCS, SAD1 or 2, GOS2 or any promoter that gives enhanced expression.

A “strong promoter” refers to a promoter that leads to increased or overexpression of the gene. Examples of strong promoters include, but are not limited to, CaMV-35S, CaMV-35Somega, Arabidopsis ubiquitin UBQ1, rice ubiquitin, actin, or Maize alcohol dehydrogenase 1 promoter (Adh-1).

The term “operably linked” as used herein refers to a functional linkage between the promoter sequence and the gene of interest, such that the promoter sequence is able to initiate transcription of the gene of interest.

In one embodiment, the progeny plant is stably transformed with the nucleic acid construct described herein and comprises the exogenous polynucleotide which is heritably maintained in the plant cell. The method may include steps to verify that the construct is stably integrated. The method may also comprise the additional step of collecting seeds from the selected progeny plant.

In another embodiment, the method increases resistance to lodging in a plant by decreasing the expression or activity of miR528.

miR528 is a monocot-specific miRNA. In rice, miR528 targets an L-ascorbate oxidase (AO), a plastocyanin-like protein, a RING-H2 finger E3 ubiquitin ligase VirE2-interacting protein 2, and an F-box domain and leucine-rich repeat-containing protein DWARF3 (Wu et al., 2017). When rice is infected by viruses, miR528 preferentially associates with AGO18, resulting in enhanced AO activity, higher basal reactive oxygen species accumulation, and enhanced antiviral defence (Wu et al., 2017). Constitutive expression of rice miR528 enhances tolerance to salinity stress and N starvation in creeping bentgrass by repressing AAO (ascorbate acid oxidase) and CBP1 (copper ion binding protein 1) transcripts (Yuan et al., 2015). However, the functional significance of miR528 in maize remained unclear because the predicted potential targets of ZmmiR528 are different from those in rice. In addition, in maize there are two members of miR528—miR528a and miR528b each encoded by a different locus. Each locus produces a miR528 with a different precursor sequence (shown as SEQ ID NO: 32 and 39 herein) although the mature sequence produced are identical (the RNA and DNA sequence of mature miR528 is shown in SEQ ID NO: 9 and 10 respectively). Here we show that lignin composition and content in maize are significantly affected by N supply and that ZmLACCASE3 (ZmLAC3) and ZmLACCASE5 (ZmLAC5) are the authentic targets of ZmmiR528. We also demonstrate that ZmmiR528, by negatively regulating the abundance of ZmLAC3 and ZmLAC5 mRNA, affects maize lignin biosynthesis and lodging resistance.

By “decreasing expression” of miR528 is meant a decrease in the nucleotide (DNA or RNA) levels of miR528 compared to a control plant. In one example, the activity of miR528 can be assessed by measuring laccase 3 and/5 protein or RNA levels. In one embodiment, the expression or activity of at least one miR528 are decreased by up to or more than 5%, 10%, 20%, 30%, 40%, 50%, 60%, 70%, 80% or 90% when compared to the level in a wild-type or control plant. By “abolished” is meant that no miR528 is expressed or can be detectably expressed. Methods for determining miR528 nucleotide expression would be well known to the skilled person. In particular decreases can be measured by any standard technique known to the skilled person. For example, a decrease in the expression levels may comprise a measure of nucleic acid levels and can be measured by any technique known to the skilled person, such as, but not limited to, any form of gel electrophoresis or chromatography (e.g. HPLC).

In one embodiment, the method may comprise introducing at least one mutation into at least one miR528 gene (such as at least one precursor sequence or the mature miR528 sequence as described herein, for example in SEQ ID NOs 32, 39, 49 or 50) and/or promoter sequence such that miR528 is either not expressed (i.e. expression is abolished) or expression is reduced, as defined herein. Alternatively, at least one mutation may be introduced into the miR528 such that the altered gene does not express a functional product—i.e. it is incapable of binding to LAC3 and/or LAC5. In this manner, the activity of miR528 can be considered to be reduced or abolished. Preferably expression of miR528 is abolished. In this manner the mutation is a knockout mutation.

In an alternative embodiment, decreasing the activity of miR528 may involve mutating the site at which miR528 binds to its targets—LAC3 and LAC5 leaving miR528 unable to bind and degrade LAC3 and/or LAC5 mRNA. This in turn will lead to an increase in the protein levels of LAC3 and LAC5.

Accordingly, in one embodiment, the method comprises introducing at least one mutation into the miR528 binding site of at least one laccase gene. Most preferably, the method comprises introducing at least one mutation into the miR528 binding site of LAC3 and/or LAC5. In one embodiment a mutation is introduced into both the LAC3 and LAC5 miR528 binding site.

The miR528 binding site in LAC3 is as follows:

(SEQ ID NO: 20)
CUCUGCUGCAUGCCCCUUCGA

The miR528 binding site in LAC5 is as follows:

(SEQ ID NO: 21)
CUUCUCCGCAUGUCCCUUCCU

Preferably, the mutation is any mutation that prevents the binding of miR528 to LAC3 and/or LAC5. In one example, the mutation may be selected from a deletion, insertion and substitution of one or more nucleotides in SEQ ID NO: 20 and/or 21 described above. In one example, 1, 2, 3, 4, 5, 6, 7, 8, 9, 10, 11, 12, 13, 14, 15, 16, 17, 18, 19 or 20 nucleotides are deleted or inserted. In one embodiment the mutation is a silent mutation. More preferably the mutation also does not affect the laccase (e.g. oxidase) activity of the protein.

In one embodiment, the mutation is introduced using mutagenesis or targeted genome editing. That is, in one embodiment, the invention relates to a method and plant that has been generated by genetic engineering methods as described above, and does not encompass naturally occurring varieties.

Targeted genome modification or targeted genome editing is a genome engineering technique that uses targeted DNA double-strand breaks (DSBs) to stimulate genome editing through homologous recombination (HR)-mediated recombination events. To achieve effective genome editing via introduction of site-specific DNA DSBs, four major classes of customizable DNA binding proteins can be used: meganucleases derived from microbial mobile genetic elements, ZF nucleases based on eukaryotic transcription factors, transcription activator-like effectors (TALEs) from Xanthomonas bacteria, and the RNA-guided DNA endonuclease Cas9 from the type II bacterial adaptive immune system CRISPR (clustered regularly interspaced short palindromic repeats). Meganuclease, ZF, and TALE proteins all recognize specific DNA sequences through protein-DNA interactions. Although meganucleases integrate nuclease and DNA-binding domains, ZF and TALE proteins consist of individual modules targeting 3 or 1 nucleotides (nt) of DNA, respectively. ZFs and TALEs can be assembled in desired combinations and attached to the nuclease domain of Fokl to direct nucleolytic activity toward specific genomic loci.

Upon delivery into host cells via the bacterial type III secretion system, TAL effectors enter the nucleus, bind to effector-specific sequences in host gene promoters and activate transcription. Their targeting specificity is determined by a central domain of tandem, 33-35 amino acid repeats. This is followed by a single truncated repeat of 20 amino acids. The majority of naturally occurring TAL effectors examined have between 12 and 27 full repeats.

These repeats only differ from each other by two adjacent amino acids, their repeat-variable di-residue (RVD). The RVD that determines which single nucleotide the TAL effector will recognize: one RVD corresponds to one nucleotide, with the four most common RVDs each preferentially associating with one of the four bases. Naturally occurring recognition sites are uniformly preceded by a T that is required for TAL effector activity. TAL effectors can be fused to the catalytic domain of the Fokl nuclease to create a TAL effector nuclease (TALEN) which makes targeted DNA double-strand breaks (DSBs) in vivo for genome editing. The use of this technology in genome editing is well described in the art, for example in U.S. Pat. Nos. 8,440,431, 8,440,432 and 8,450,471. Cermak T et al. describes a set of customized plasmids that can be used with the Golden Gate cloning method to assemble multiple DNA fragments. As described therein, the Golden Gate method uses Type IIS restriction endonucleases, which cleave outside their recognition sites to create unique 4 bp overhangs. Cloning is expedited by digesting and ligating in the same reaction mixture because correct assembly eliminates the enzyme recognition site. Assembly of a custom TALEN or TAL effector construct and involves two steps: (i) assembly of repeat modules into intermediary arrays of 1-10 repeats and (ii) joining of the intermediary arrays into a backbone to make the final construct. Accordingly, using techniques known in the art it is possible to design a TAL effector that targets the miR528 gene or promoter or the miR528 binding sequence in LAC3 and/or LAC5 as described herein.

Another genome editing method that can be used according to the various aspects of the invention is CRISPR. The use of this technology in genome editing is well described in the art, for example in U.S. Pat. No. 8,697,359 and references cited herein. In short, CRISPR is a microbial nuclease system involved in defence against invading phages and plasmids. CRISPR loci in microbial hosts contain a combination of CRISPR-associated (Cas) genes as well as non-coding RNA elements capable of programming the specificity of the CRISPR-mediated nucleic acid cleavage (sgRNA). Three types (I-III) of CRISPR systems have been identified across a wide range of bacterial hosts. One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences, which direct Cas nucleases to the target site (protospacer). The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates leavage of target DNA to create a double-stranded break within the protospacer.

One major advantage of the CRISPR-Cas9 system, as compared to conventional gene targeting and other programmable endonucleases is the ease of multiplexing, where multiple genes can be mutated simultaneously simply by using multiple sgRNAs each targeting a different gene. In addition, where two sgRNAs are used flanking a genomic region, the intervening section can be deleted or inverted (Wiles et al., 2015).

Cas9 is thus the hallmark protein of the type II CRISPR-Cas system, and is a large monomeric DNA nuclease guided to a DNA target sequence adjacent to the PAM (protospacer adjacent motif) sequence motif by a complex of two noncoding RNAs: CRISPR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can introduce site-specific double strand breaks (DSBs) into genomic DNA of live cells from various organisms. For applications in eukaryotic organisms, codon optimized versions of Cas9, which is originally from the bacterium Streptococcus pyogenes, have been used.

The single guide RNA (sgRNA) is the second component of the CRISPR/Cas system that forms a complex with the Cas9 nuclease. sgRNA is a synthetic RNA chimera created by fusing crRNA with tracrRNA. The sgRNA guide sequence located at its 5′ end confers DNA target specificity. Therefore, by modifying the guide sequence, it is possible to create sgRNAs with different target specificities. The canonical length of the guide sequence is 20 bp. In plants, sgRNAs have been expressed using plant RNA polymerase III promoters, such as U6 and U3. Accordingly, using techniques known in the art it is possible to design sgRNA molecules that target miR528a and/or b or the miR528 binding site in the LAC3 and/or LAC5 gene. Examples of such suitable CRISPR constructs that can be used according to the methods described herein are described below.

In one embodiment, the method uses the sgRNA (and template or donor DNA) constructs defined in detail below to introduce a targeted SNP or mutation into a miR528 gene. Preferably the mutation reduces or abolishes expression of the miR528. Alternatively, as a result of the mutation, miR528 is no longer able to bind to, LAC 3 and/or 5. As explained below, the introduction of a template DNA strand, following a sgRNA-mediated snip in the double-stranded DNA, can be used to produce a specific targeted mutation (i.e. a SNP) in the gene using homology directed repair.

In another example, sgRNA (for example, as described herein) can be used with a modified Cas9 protein, such as nickase Cas9 or nCas9 or a “dead” Cas9 (dCas9) fused to a “Base Editor”—such as an enzyme, for example a deaminase such as cytidine deaminase, or TadA (tRNA adenosine deaminase) or ADAR or APOBEC. These enzymes are able to substitute one base for another. As a result no DNA is deleted, but a single substitution is made (Kim et al., 2017; Gaudelli et al. 2017).

In one example, a mutation is introduced into miRNA528a and/or miRNA528b using the following sgRNA sequences as described herein, shown in SEQ ID NO: 35, 38, 42 and/or 45.

In another embodiment, the method uses a sgRNA construct as described herein to introduce at least one mutation into the miRNA528 binding site in the LAC3 and/or LAC5 gene. Alternatively the CRISPR system can be used to replace the miRNA528 binding site in LAC3 or LAC5 with an artificial or donor sequence. Preferably the artificial sequence comprises at least one mutation in the miRNA binding site that are synonymous mutations (i.e. alters the nucleic acid sequence but not the amino acid sequence). As such, miRNA528 is unable to bind or bind with less efficiency, but protein function of the laccase is unaffected. In this manner the activity of miRNA528 can be considered to be reduced, as described herein. Methods for using CRISPR to introduce targeted DNA replacements or donor sequences are known in the art (see for e.g. Zhao et al. 2016 and Zhang et al. 2018). However, in one example, there is provided a sgRNA construct, where the sgRNA construct comprises at least one nucleic acid sequence that targets (can bind to) at least one sequence selected from SEQ ID NO: 51 (LAC3), 54 (LAC3), 57 (LAC5) or and 60 (LAC5) or a variant thereof (as defined above). More preferably the sgRNA construct comprises at least one protospacer sequence wherein the protospacer sequence is selected from SEQ ID NO: 52 (LAC3), 55 (LAC3), 58 (LAC5) and 61 (LAC5). Even more preferably, the sgRNA construct comprises a nucleic acid sequence encoding a sgRNA selected from one of SEQ ID NO: 53 (LAC3), 56 (LAC3), 59 (LAC5) and 62 (LAC5) or a variant thereof. The nucleic acid sequences are preferably operably linked to a regulatory sequence, such as a promoter, examples of which are described herein. The sgRNA construct may also comprise a CRISPR enzyme, as described herein, such as Cas, preferably Cas 9 or Cpf1. In this example, where the target sequence is selected from SEQ ID NO: 51 (LAC3) or 57 (LAC5) or the protospacer sequence selected from SEQ ID NO: 52 (LAC3) or SEQ ID NO: 58 (LAC5) or the sgRNA nucleic acid sequence selected from SEQ ID NO: 53 (LAC3) or SEQ ID NO: 59 (LAC5) the CRISPR enzyme is a Cas protein, preferably Cas9. Alternatively, where the target sequence is selected from SEQ ID NO: 54 (LAC3) and 60 (LA5) or the protospacer sequence selected from SEQ ID NO: 55 (LAC3) or SEQ ID NO: 61 (LAC5) or the sgRNA nucleic acid sequence selected from SEQ ID NO: 56 (LAC3) or SEQ ID NO: 562 (LAC5) the CRISPR enzyme is Cpf1.

Furthermore, there is also provided a donor sequence construct comprising a donor sequence to replace the miRNA528 binding site in the LAC3 or LAC5 gene. In one example, the donor sequence comprises SEQ ID NO: 65, preferably where the target is LAC3. In another example the donor sequence comprises SEQ ID NO: 66, preferably where the target sequence is LAC5. In a preferred example, the donor sequence is operably linked to a regulatory sequence, such as any of the promoters described herein. However, in an alternative embodiment, the donor sequences may be present on the same construct as the sgRNA sequences and under the control of the same or separate regulatory sequences.

By “crRNA” or CRISPR RNA is meant the sequence of RNA that contains the protospacer element and additional nucleotides that are complementary to the tracrRNA.

By “tracrRNA” (transactivating RNA) is meant the sequence of RNA that hybridises to the crRNA and binds a CRISPR enzyme, such as Cas9 thereby activating the nuclease complex to introduce double-stranded breaks at specific sites within the genomic sequence of at least one miRNA528 nucleic acid or promoter sequence.

By “protospacer element” is meant the portion of crRNA (or sgRNA) that is complementary to the genomic DNA target sequence, usually around 20 nucleotides in length. This may also be known as a spacer or targeting sequence.

By “sgRNA” (single-guide RNA) is meant the combination of tracrRNA and crRNA in a single RNA molecule, preferably also including a linker loop (that links the tracrRNA and crRNA into a single molecule). “sgRNA” may also be referred to as “gRNA” and in the present context, the terms are interchangeable. The sgRNA or gRNA provide both targeting specificity and scaffolding/binding ability for a Cas or Cpf1 nuclease. A gRNA may refer to a dual RNA molecule comprising a crRNA molecule and a tracrRNA molecule.

By “donor sequence” is meant a nucleic acid sequence that contains all the necessary elements to introduce a specific substitution or sequence into a target sequence, preferably using homology-directed repair or HDR. In one embodiment, the donor sequence is flanked by at least one, preferably a left and right arm each that are identical to the target sequence. The arm or arms may also be further flanked by two gRNA target sequences that comprise PAM motifs so that the donor sequence can be released by Cas9/gRNAs.

By “TAL effector” (transcription activator-like (TAL) effector) or TALE is meant a protein sequence that can bind the genomic DNA target sequence (e.g. a sequence within the miRNA528 gene or promoter sequence or miR528 binding site in LAC3 or LAC5) and that can be fused to the cleavage domain of an endonuclease such as Fokl to create TAL effector nucleases or TALENS or meganucleases to create megaTALs. A TALE protein is composed of a central domain that is responsible for DNA binding, a nuclear-localisation signal and a domain that activates target gene transcription. The DNA-binding domain consists of monomers and each monomer can bind one nucleotide in the target nucleotide sequence. Monomers are tandem repeats of 33-35 amino acids, of which the two amino acids located at positions 12 and 13 are highly variable (repeat variable diresidue, RVD). It is the RVDs that are responsible for the recognition of a single specific nucleotide. HD targets cytosine; NI targets adenine, NG targets thymine and NN targets guanine (although NN can also bind to adenine with lower specificity).

In another aspect of the invention there is provided a nucleic acid construct where the nucleic acid construct encodes at least one DNA-binding domain, wherein the DNA-binding domain can bind to a sequence in a miRNA528 gene, wherein said sequence is selected from SEQ ID NO: 33, 36, 40 or 43. In a further aspect of the invention, there is provided a nucleic acid construct that encodes at least one DNA-binding domain, where the DNA-binding domain can bind to a sequence in a LAC3 or LAC5 gene, where preferably the sequence is selected from SEQ ID NO: 51 (LAC3), 54 (LAC3), 57 (LAC5) and 60 (LAC5).

In one embodiment, said construct further comprises a nucleic acid encoding a (SSN) sequence-specific nuclease, such as Fokl or a CRISPR enzyme such as a Cas or Cpf1 protein.

In one embodiment, the nucleic acid construct encodes at least one protospacer element wherein the sequence of the protospacer element is selected from SEQ ID NO 34, 37, 41, 44, or a variant thereof. Alternatively, the at least one protospacer element is selected from SEQ ID NO: 52, 55, 58 and 61 or a variant thereof.

In a further embodiment, the nucleic acid construct comprises a crRNA-encoding sequence. As defined above, a crRNA sequence may comprise the protospacer elements as defined above and preferably additional nucleotides that are complementary to the tracrRNA. An appropriate sequence for the additional nucleotides will be known to the skilled person as these are defined by the choice of Cas or Cpf1 protein. In one example however, the sequence of the crRNA or additional nucleotides sequence comprises SEQ ID NO: 48 or a variant thereof.

In another embodiment, the nucleic acid construct further comprises a tracrRNA sequence. Again, an appropriate tracrRNA sequence would be known to the skilled person as this sequence is defined by the choice of Cas protein. Nonetheless, in one example said sequence comprises or consists of a sequence as defined in SEQ ID NO: 31 or a variant thereof.

In a further embodiment, the nucleic acid construct comprises at least one nucleic acid sequence that encodes a sgRNA (or gRNA). Again, as already discussed, sgRNA typically comprises a crRNA sequence, a tracrRNA sequence and preferably a sequence for a linker loop. In one example, the sgRNA sequence comprises SEQ ID NO: 48 or a variant thereof, and preferably a protospacer sequence, such as any of the sequences defined as such herein. In a preferred embodiment, the nucleic acid construct comprises at least one nucleic acid sequence that encodes a sgRNA sequence as defined in any of SEQ ID NO: 35, 38, 42, 45, or variant thereof. In another preferred embodiment, the nucleic acid construct comprises at least one nucleic acid sequence that encodes a sgRNA sequence as defined in any of SEQ ID NO: 53, 56, 59 and 62 or a variant thereof.

In a further embodiment, the nucleic acid construct may further comprise at least one nucleic acid sequence encoding an endoribonuclease cleavage site. Preferably the endoribonuclease is Csy4 (also known as Cas6f). Where the nucleic acid construct comprises multiple sgRNA nucleic acid sequences the construct may comprise the same number of endoribonuclease cleavage sites. In another embodiment, the cleavage site is 5′ of the sgRNA nucleic acid sequence. Accordingly, each sgRNA nucleic acid sequence is flanked by an endoribonuclease cleavage site.

The term ‘variant’ as used throughout refers to a nucleotide sequence where the nucleotides are substantially identical to one of the above sequences. The variant may be achieved by modifications such as insertion, substitution or deletion of one or more nucleotides. In a preferred embodiment, the variant has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to any one of the above described sequences. In one embodiment, sequence identity is at least 90%. In another embodiment, sequence identity is 100%. Sequence identity can be determined by any one known sequence alignment program in the art.

The invention also relates to a nucleic acid construct comprising one of these nucleic acid sequences operably linked to a suitable plant promoter. A suitable plant promoter may be a constitutive or strong promoter or may be a tissues-specific promoter. In one embodiment, suitable plant promoters are selected from, but not limited to, cestrum yellow leaf curling virus (CmYLCV) promoter or switchgrass ubiquitin 1 promoter (PvUbi1) wheat U6 RNA polymerase III (TaU6) CaMV35S, wheat U6 or maize ubiquitin (e.g. Ubi1) promoters. In one embodiment, the promoter is ZmUbi (SEQ ID NO: 46).

The nucleic acid construct of the present invention may also further comprise a nucleic acid sequence that encodes a CRISPR enzyme. By “CRISPR enzyme” is meant an RNA-guided DNA endonuclease that can associate with the CRISPR system. Specifically, such an enzyme binds to the tracrRNA sequence. In one embodiment, the CRIPSR enzyme is a Cas protein (“CRISPR associated protein), preferably Cas 9 or Cpf1, more preferably Cas9. In a specific embodiment Cas9 is codon-optimised Cas9, and more preferably, has the sequence described in SEQ ID NO: 47 or a functional variant or homolog thereof. In an alternative embodiment, the CRISPR enzyme is Cpf1 and comprises a nucleic acid sequence that encodes a Cpf1 protein as defined in SEQ ID NO: 64 or a functional variant or homolog thereof. More preferably, the Cpf1 sequence comprises or consists of SEQ ID NO: 63 or a functional variant or homolog thereof. In another embodiment, the CRISPR enzyme is a protein from the family of Class 2 candidate proteins, such as C2c1, C2C2 and/or C2c3. In one embodiment, the Cas protein is from Streptococcus pyogenes. In an alternative embodiment, the Cas protein may be from any one of Staphylococcus aureus, Neisseria meningitides, Streptococcus thermophiles or Treponema denticola.

The term “functional variant” as used herein with reference to Cas9 or Cpf1 refers to a variant Cas9 or Cpf1 gene sequence or part of the gene sequence which retains the biological function of the full non-variant sequence, for example, acts as a DNA endonuclease, or recognition or/and binding to DNA. A functional variant also comprises a variant of the gene of interest which has sequence alterations that do not affect function, for example non-conserved residues. Also encompassed is a variant that is substantially identical, i.e. has only some sequence variations, for example in non-conserved residues, compared to the wild type sequences as shown herein and is biologically active. In one embodiment, a functional variant of SEQ ID NO.47 or 63 has at least 90%, 91%, 92%, 93%, 94%, 95%, 96%, 97%, 98%, or 99% overall sequence identity to the amino acid represented by SEQ ID NO: 47 or 63. In a further embodiment, the Cas9 protein has been modified to improve activity.

Suitable homologs or orthologs can be identified by sequence comparisons and identifications of conserved domains. The function of the homolog or ortholog can be identified as described herein and a skilled person would thus be able to confirm the function when expressed in a plant.

In a further embodiment, the Cas9 protein has been modified to improve activity. For example, in one embodiment, the Cas9 protein may comprise the D10A amino acid substitution, this nickase cleaves only the DNA strand that is complementary to and recognized by the gRNA. In an alternative embodiment, the Cas9 protein may alternatively or additionally comprise the H840A amino acid substitution, this nickase cleaves only the DNA strand that does not interact with the sRNA. In this embodiment, Cas9 may be used with a pair (i.e. two) sgRNA molecules (or a construct expressing such a pair) and as a result can cleave the target region on the opposite DNA strand, with the possibility of improving specificity by 100-1500 fold. In a further embodiment, the Cas9 protein may comprise a D1135E substitution. The Cas9 protein may also be the VQR variant. Alternatively, the Cas protein may comprise a mutation in both nuclease domains, HNH and RuvC-like and therefore is catalytically inactive. Rather than cleaving the target strand, this catalytically inactive Cas protein can be used to prevent the transcription elongation process, leading to a loss of function of incompletely translated proteins when co-expressed with a sgRNA molecule. An example of a catalytically inactive protein is dead Cas9 (dCas9) caused by a point mutation in RuvC and/or the HNH nuclease domains (Komor et al., 2016 and Nishida et al., 2016).

In a further embodiment, a Cas protein, such as Cas9 may be further fused with a repression effector, such as a histone-modifying/DNA methylation enzyme or a Base Editor, such as cytidine deaminase (Komor et al. 2016) to effect site-directed mutagenesis, as described above. In the latter, the cytidine deaminase enzyme does not induce dsDNA breaks, but mediates the conversion of cytidine to uridine, thereby effecting a C to T (or G to A) substitution.

In a further embodiment, the nucleic acid construct comprises an endoribonuclease. Preferably the endoribonuclease is Csy4 (also known as Cas6f) and more preferably a codon optimised csy4. In one embodiment, where the nucleic acid construct comprises a cas protein, the nucleic acid construct may comprise sequences for the expression of an endoribonuclease, such as Csy4 expressed as a 5′ terminal P2A fusion (used as a self-cleaving peptide) to a cas protein, such as Cas9.

In one embodiment, the cas protein, the endoribonuclease and/or the endoribonuclease-cas fusion sequence may be operably linked to a suitable plant promoter. Suitable plant promoters are already described above, but in one embodiment, may be the Zea Mays Ubiquitin 1 promoter.

Suitable methods for producing the CRISPR nucleic acids and vectors system are known, and for example are published in Molecular Plant (Ma et al., 2015, Molecular Plant, DOI:10.1016/j.molp.2015.04.007), which is incorporated herein by reference.

In an alternative aspect of the invention, the nucleic acid construct comprises at least one nucleic acid sequence that encodes a TAL effector, wherein said effector targets a miRNA528 sequence selected from SEQ ID NO: 33, 36, 40 or 43 or a miRNA528 binding site in LAC3 selected from SEQ ID NO: 51 and 54 or a miRNA528 binding site in LAC5 selected from SEQ ID NO: 57 and 60. Methods for designing a TAL effector would be well known to the skilled person, given the target sequence. Examples of suitable methods are given in Sanjana et al., and Cermak T et al., both incorporated herein by reference. Preferably, said nucleic acid construct comprises two nucleic acid sequences encoding a TAL effector, to produce a TALEN pair. In a further embodiment, the nucleic acid construct further comprises a sequence-specific nuclease (SSN). Preferably such SSN is an endonuclease such as Fokl. In a further embodiment, the TALENs are assembled by the Golden Gate cloning method in a single plasmid or nucleic acid construct.

In another aspect of the invention, there is provided a sgRNA molecule, wherein the sgRNA molecule comprises a crRNA sequence and a tracrRNA sequence and wherein the crRNA sequence can bind to at least one target sequence selected from SEQ ID NO: 33, 36, 40 or 43 or a variant thereof. Preferably, the sgRNA molecule has a nucleic acid sequence comprising SEQ ID NO: 35, 38, 42 and 45 and a RNA sequence selected from SEQ ID NO: 72 to 75.

Alternatively, there is provided a sgRNA molecule, where the sgRNA molecule can bind to at least one target sequence selected from SEQ ID NO: 51, 54, 57 and 60. Preferably, the sgRNA molecule has a nucleic acid sequence comprising SEQ ID NO: 53, 56, 59 and 62 and a RNA sequence selected from SEQ ID NO: 76 to 79.

A “variant” is as defined herein. In one embodiment, the sgRNA molecule may comprise at least one chemical modification, for example that enhances its stability and/or binding affinity to the target sequence or the crRNA sequence to the tracrRNA sequence. Such modifications would be well known to the skilled person, and include for example, but not limited to, the modifications described in Rahdar et al., 2015, incorporated herein by reference. In this example the crRNA may comprise a phosphorothioate backbone modification, such as 2′-fluoro (2′-F), 2′-O-methyl (2′-O-Me) and S-constrained ethyl (cET) substitutions.

In another aspect of the invention, there is provided an isolated nucleic acid sequence that encodes for a protospacer element (as defined in any of SEQ ID NO: 34, 37, 41, 44, or a variant thereof).

In another aspect of the invention, there is provided a plant or part thereof or at least one isolated plant cell transfected with at least one nucleic acid construct as described herein. Cas9 and sgRNA may be combined or in separate expression vectors (or nucleic acid constructs, such terms are used interchangeably). In other words, in one embodiment, an isolated plant cell is transfected with a single nucleic acid construct comprising both sgRNA and Cas9 as described in detail above. In an alternative embodiment, an isolated plant cell is transfected with two nucleic acid constructs, a first nucleic acid construct comprising at least one sgRNA as defined above and a second nucleic acid construct comprising Cas9 or a functional variant or homolog thereof. The second nucleic acid construct may be transfected below, after or concurrently with the first nucleic acid construct. The advantage of a separate, second construct comprising a cas protein is that the nucleic acid construct encoding at least one sgRNA can be paired with any type of cas protein, as described herein, and therefore are not limited to a single cas function (as would be the case when both cas and sgRNA are encoded on the same nucleic acid construct).

In another aspect of the invention, there is provided a plant or part thereof or at least one isolated plant cell transfected with a single nucleic acid construct comprising both sgRNA and Cas9 or sgRNA, Cas9 and the donor DNA sequence as described in detail above. In an alternative embodiment, an isolated plant cell is transfected with two or three nucleic acid constructs, a first nucleic acid construct comprising at least one sgRNA as defined above, a second nucleic acid construct comprising Cas9 or a functional variant or homolog thereof and a third nucleic acid construct comprising the donor DNA sequence as defined above. Again, the second and/or third nucleic acid construct may be transfected before, after or concurrently with the first and/or second nucleic acid construct.

In one embodiment, the nucleic acid construct comprising a CRSIPR enzyme is transfected first and is stably incorporated into the genome, before the second transfection with a nucleic acid construct comprising at least one sgRNA nucleic acid. In an alternative embodiment, a plant or part thereof or at least one isolated plant cell is transfected with mRNA encoding a Cas protein and co-transfected with at least one nucleic acid construct as defined herein.

Cas9 expression vectors for use in the present invention can be constructed as described in the art. In one example, the expression vector comprises a nucleic acid sequence as defined in SEQ ID NO: 47 or a functional variant thereof, wherein said nucleic acid sequence is operably linked to a suitable promoter. Examples of suitable promoters include the Actin, CaMV35S, wheat U6 or maize ubiquitin (e.g. Ubi1) promoter.

Also included in the scope of the invention is the use of the nucleic acid constructs (CRISPR constructs) described above or the sgRNA molecules in any of the above described methods. For example, there is provided the use of the above CRISPR constructs or sgRNA molecules to reduce miRNA528 expression or activity as described herein.

Therefore, in a further aspect of the invention, there is provided a method of decreasing miRNA528 expression and/or activity, the method comprising introducing and expressing any one of the above described constructs or introducing a sgRNA molecule as also described above into a plant. In other words, there is also provided a method of decreasing miRNA528 expression and/or activity, as described herein, wherein the method comprises introducing at least one mutation into the endogenous miRNA528 gene and/or promoter or into the miRNA528 binding site in the LAC3 and/or LAC5 gene using CRISPR/Cas9, and specifically, the CRISPR (nucleic acid) constructs described herein.

In an alternative aspect of the present invention, there is provided an isolated plant cell transfected with at least one sgRNA molecule as described herein.

In a further aspect of the invention, there is provided a genetically modified or edited plant comprising the transfected cell described herein. In one embodiment, the nucleic acid construct or constructs may be integrated in a stable form. In an alternative embodiment, the nucleic acid construct or constructs are not integrated (i.e. are transiently expressed). Accordingly, in a preferred embodiment, the genetically modified plant is free of any sgRNA and/or Cas/Cpf1 protein nucleic acid. In other words, the plant is transgene free.

The terms “introduction”, “transfection” or “transformation” as referred to herein encompass the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The resulting transformed plant cell may then be used to regenerate a transformed plant in a manner known to persons skilled in the art. The transfer of foreign genes into the genome of a plant is called transformation. Transformation of plants is now a routine technique in many species. Any of several transformation methods known to the skilled person may be used to introduce the nucleic acid construct or sgRNA molecule of interest into a suitable ancestor cell. The methods described for the transformation and regeneration of plants from plant tissues or plant cells may be utilized for transient or for stable transformation.

Transformation methods include the use of liposomes, electroporation, chemicals that increase free DNA uptake, injection of the DNA directly into the plant (microinjection), gene guns (or biolistic particle delivery systems (biolistics)) as described in the examples, lipofection, transformation using viruses or pollen and microprojection. Methods may be selected from the calcium/polyethylene glycol method for protoplasts, ultrasound-mediated gene transfection, optical or laser transfection, transfection using silicon carbide fibres, electroporation of protoplasts, microinjection into plant material, DNA or RNA-coated particle bombardment, infection with (non-integrative) viruses and the like. Transgenic plants can also be produced via Agrobacterium tumefaciens mediated transformation, including but not limited to using the floral dip/Agrobacterium vacuum infiltration method as described in Clough & Bent (1998) and incorporated herein by reference.

Accordingly, in one embodiment, at least one nucleic acid construct or sgRNA molecule as described herein can be introduced to at least one plant cell using any of the above described methods. In an alternative embodiment, any of the nucleic acid constructs described herein may be first transcribed to form a preassembled Cas9-sgRNA ribonucleoprotein and then delivered to at least one plant cell using any of the above described methods, such as lipofection, electroporation or microinjection.

Optionally, to select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility is growing the seeds, if appropriate after sterilization, on agar plates using a suitable selection agent so that only the transformed seeds can grow into plants. As described in the examples, a suitable marker can be bar-phosphinothricin or PPT. Alternatively, the transformed plants are screened for the presence of a selectable marker, such as, but not limited to, GFP, GUS (β-glucuronidase). Other examples would be readily known to the skilled person. Alternatively, no selection is performed, and the seeds obtained in the above-described manner are planted and grown and miRNA528 expression, protein levels or binding to LAC3 or LAC5 measured at an appropriate time using standard techniques in the art. This alternative, which avoids the introduction of transgenes, is preferable to produce transgene-free plants.

Following DNA transfer and regeneration, putatively transformed plants may also be evaluated, for instance using PCR to detect the presence of the gene of interest, copy number and/or genomic organisation. Alternatively or additionally, integration and expression levels of the newly introduced DNA may be monitored using Southern, Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques.

Specific protocols for using the above described CRISPR constructs would be well known to the skilled person. As one example, a suitable protocol is described in Ma & Liu (“CRISPR/Cas-based multiplex genome editing in monocot and dicot plants”) incorporated herein by reference.

In a further related aspect of the invention, there is also provided, a method of obtaining a genetically modified plant as described herein, the method comprising
a. selecting a part of the plant;
b. transfecting at least one cell of the part of the plant of paragraph (a) with at least one nucleic acid construct as described herein or at least one sgRNA molecule as described herein, using the transfection or transformation techniques described above;
c. regenerating at least one plant derived from the transfected cell or cells;
d. selecting one or more plants obtained according to paragraph (c) that show reduced expression or function of miRNA528 or a laccase 3 or 5 that hows reduced binding of miR528.

In a further embodiment, the method also comprises the step of screening the genetically modified plant for SSN (preferably CRISPR)-induced mutations in the miRNA528 gene or promoter sequence or in the miR528 binding site of LAC3 or LAC5. In one embodiment, the method comprises obtaining a DNA sample from a transformed plant and carrying out DNA amplification to detect a mutation in at least one miRNA528 gene or promoter sequence.

In a further embodiment, the methods comprise generating stable T2 plants preferably homozygous for the mutation (that is a mutation in at least one miRNA528 gene or promoter sequence or in the miR528 binding site of LAC3 or LAC5).

Plants that have a mutation in at least one miRNA528 gene sequence or in the miR528 binding site of LAC3 or LAC5 can also be crossed with another plant also containing at least one different mutation in at least one miRNA528 gene or in the miR528 binding site of LAC3 or LAC5 sequence to obtain plants with additional mutations in the miRNA528 gene sequence. The combinations will be apparent to the skilled person. Accordingly, this method can be used to generate a T2 plant with mutations on all or an increased number of homoeologs, when compared to the number of homoeolog mutations in a single T1 plant transformed as described above.

A plant obtained or obtainable by the methods described above is also within the scope of the invention.

A genetically altered plant of the present invention may also be obtained by transference of any of the sequences of the invention by crossing, e.g., using pollen of the genetically altered plant described herein to pollinate a wild-type or control plant, or pollinating the gynoecia of plants described herein with other pollen that does not contain a mutation in at least one of the miRNA528 gene or promoter sequence or in the miR528 binding site of LAC3 or LAC5. The methods for obtaining the plant of the invention are not exclusively limited to those described in this paragraph; for example, genetic transformation of germ cells from the ear of wheat could be carried out as mentioned, but without having to regenerate a plant afterwards.

Alternatively, more conventional mutagenesis methods can be used to introduce at least one mutation into the miR528 gene and/or promoter or the LAC3 and/or LAC5 miR528 binding sequence. These methods include both physical and chemical mutagenesis. A skilled person will know further approaches can be used to generate such mutants, and methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein.

In one embodiment, insertional mutagenesis is used, for example using T-DNA mutagenesis (which inserts pieces of the T-DNA from the Agrobacterium tumefaciens T-Plasmid into DNA causing either loss of gene function or gain of gene function mutations), site-directed nucleases (SDNs) or transposons as a mutagen. Insertional mutagenesis is an alternative means of disrupting gene function and is based on the insertion of foreign DNA into the gene of interest (see Krysan et al, The Plant Cell, Vol. 11, 2283-2290, December 1999). Accordingly, in one embodiment, T-DNA is used as an insertional mutagen to disrupt the miR528 a or b gene or miR528 promoter or the LAC3 and/or LAC5 miR528 binding sequence such that miR528 cannot bind. An example of using T-DNA mutagenesis to disrupt the Arabidopsis ARE1 gene is described in Downes et al. 2003. T-DNA not only disrupts the expression of the gene into which it is inserted, but also acts as a marker for subsequent identification of the mutation. Since the sequence of the inserted element is known, the gene in which the insertion has occurred can be recovered, using various cloning or PCR-based strategies. The insertion of a piece of T-DNA in the order of 5 to 25 kb in length generally produces a disruption of gene function. If a large enough population of T-DNA transformed lines is generated, there are reasonably good chances of finding a transgenic plant carrying a T-DNA insert within any gene of interest. Transformation of spores with T-DNA is achieved by an Agrobacterium-mediated method which involves exposing plant cells and tissues to a suspension of Agrobacterium cells.

The details of this method are well known to a skilled person. In short, plant transformation by Agrobacterium results in the integration into the nuclear genome of a sequence called T-DNA, which is carried on a bacterial plasmid. The use of T-DNA transformation leads to stable single insertions. Further mutant analysis of the resultant transformed lines is straightforward and each individual insertion line can be rapidly characterized by direct sequencing and analysis of DNA flanking the insertion.

In another embodiment, mutagenesis is physical mutagenesis, such as application of ultraviolet radiation, X-rays, gamma rays, fast or thermal neutrons or protons. The targeted population can then be screened to identify plants with a mutation in miR528 binding site.

In another embodiment of the various aspects of the invention, the method comprises mutagenizing a plant population with a mutagen. The mutagen may be a fast neutron irradiation or a chemical mutagen, for example selected from the following non-limiting list: ethyl methanesulfonate (EMS), methylmethane sulfonate (MMS), N-ethyl-N-nitrosurea (ENU), triethylmelamine (1′EM), N-methyl-N-nitrosourea (MNU), procarbazine, chlorambucil, cyclophosphamide, diethyl sulfate, acrylamide monomer, melphalan, nitrogen mustard, vincristine, dimethylnitosamine, N-methyl-N′-nitro-Nitrosoguanidine (MNNG), nitrosoguanidine, 2-aminopurine, 7,12 dimethyl-benz(a)anthracene (DMBA), ethylene oxide, hexamethylphosphoramide, bisulfan, diepoxyalkanes (diepoxyoctane (DEO), diepoxybutane (BEB), and the like), 2-methoxy-6-chloro-9 [3-(ethyl-2-chloroethyl)aminopropylamino]acridine dihydrochloride (ICR-170) or formaldehyde. Again, the targeted population can then be screened to identify plants with a mutation in the miR528 a or b gene or miR528 promoter of the miR528 binding site in LAC3 or LAC5.

In another embodiment, the method used to create and analyse mutations is targeting induced local lesions in genomes (TILLING), reviewed in Henikoff et al, 2004. In this method, seeds are mutagenized with a chemical mutagen, for example EMS. The resulting M1 plants are self-fertilized and the M2 generation of individuals is used to prepare DNA samples for mutational screening. DNA samples are pooled and arrayed on microtiter plates and subjected to gene specific PCR. The PCR amplification products may be screened for mutations in the in the miR528 a or b gene or miR528 promoter or the miR528 binding site using any method that identifies heteroduplexes between wild type and mutant genes. For example, but not limited to, denaturing high pressure liquid chromatography (dHPLC), constant denaturant capillary electrophoresis (CDCE), temperature gradient capillary electrophoresis (TGCE), or by fragmentation using chemical cleavage. Preferably the PCR amplification products are incubated with an endonuclease that preferentially cleaves mismatches in heteroduplexes between wild type and mutant sequences. Cleavage products are electrophoresed using an automated sequencing gel apparatus, and gel images are analyzed with the aid of a standard commercial image-processing program. Any primer specific to miR528 or LAC3/LAC5 may be utilized to amplify the miR528 or LAC3/LAC5 nucleic acid sequence within the pooled DNA sample. To facilitate detection of PCR products on a gel, the PCR primer may be labelled using any conventional labelling method. In an alternative embodiment, the method used to create and analyse mutations is EcoTILLING. EcoTILLING is molecular technique that is similar to TILLING, except that its objective is to uncover natural variation in a given population as opposed to induced mutations. The first publication of the EcoTILLING method was described in Comai et al. 2004.

Rapid high-throughput screening procedures thus allow the analysis of amplification products for identifying a mutation conferring resistance to miR528 binding or reduced miR528 expression as compared to a corresponding non-mutagenized wild type plant. Once a mutation is identified in a gene of interest, the seeds of the M2 plant carrying that mutation are grown into adult M3 plants and screened.

Plants obtained or obtainable by such method which carry a mutation in the miR528 a or b gene or miR528 promoter or the miR528 binding site of LAC3 and/or LAC5 are also within the scope of the invention

In a further embodiment, the method may comprise decreasing the expression or activity of miR528 using a miR528 inhibitor. A miR528 inhibitor is any molecule that can decrease the expression or reduce the activity of miR528. In one embodiment, the miR528 inhibitor is a nucleic acid-based molecule that suppresses miRNA function. Synthetic miRNA inhibitors can include RNA molecules that have a sequence that is the reverse complement of the mature miRNA and furthermore, are chemically modified to prevent RISC-induced cleavage, enhance binding affinity and provide resistance to nucleolytic degradation. When a synthetic miRNA inhibitor binds to miRNA528 its binding is irreversible, thus the inhibitor actually sequesters the endogenous miRNA making it unavailable for normal function (Robertson et al. 2010). Thus, in one embodiment, the miR528 inhibitor decreases activity of miRNA528 by binding and sequestering the miRNA. In another embodiment, miRNA expression can be decreased through gene editing, as described herein, where miR528 single knockout plants are generated by gene editing and then crossed to produce a miR528 double knockout plant.

In one embodiment, the miR528 inhibitor is a short tandem target mimic and comprises an RNA sequence as defined in SEQ ID NO: 8 or a functional variant thereof (as defined herein) and a DNA sequence as defined in SEQ NO: 7 or a functional variant thereof (again as defined herein).

Accordingly, in one embodiment, the method comprises introducing and expressing a nucleic acid construct comprising a nucleic acid sequence as defined in SEQ ID NO: 7 or 16 or a functional variant thereof operably linked to a regulatory sequence. In another embodiment, the method comprises introducing an RNA molecule as defined in SEQ ID NO: 8 or a functional variant thereof.

In another aspect of the invention, there is provided an isolated nucleic acid encoding a miR528 inhibitor, wherein the miR528 inhibitor comprises a nucleic acid sequence as defined in SEQ ID NO: 7 or 16 or a functional variant thereof, as defined herein.

In a further aspect, there is provided an miR528 inhibitor comprising an RNA molecule, wherein the RNA molecule comprises an RNA sequence as defined in SEQ ID NO: 8 or a functional variant thereof.

In a further aspect there is provided a nucleic acid construct comprising a nucleic acid sequence encoding a miR528 inhibitor as described above operably linked to a regulatory sequence.

Also provided is an isolated cell, preferably a plant cell plant cell or an Agrobacterium tumefaciens cell, expressing a nucleic acid construct comprising a nucleic acid sequence encoding a miR528 inhibitor or a functional variant thereof operably linked to a regulatory sequence. Furthermore, the invention also relates to a culture medium comprising an isolated plant cell or an Agrobacterium tumefaciens cell expressing a nucleic acid construct or miR528 inhibitor of the invention.

There is also provided the use of the isolated nucleic acid, miR528 inhibitor, nucleic acid construct or vector described above to increase at least one of lodging resistance, lignin content and/or synthesis in a plant compared to a control or wild-type plant. Lignin content and/or synthesis may be increased in the stems and/or roots of the plant.

In an alternative embodiment, the method decreases resistance to lodging in a plant—i.e. lodging is increased. Preferably, the method comprises decreasing the expression of at least one laccase gene and/or increasing the expression or activity of miR528. As discussed above, decreasing lodging can be useful where the plant is to be used as a source of raw material for bioenergy use or where the plant is to be used as forage for livestock. Preferably, such plants are characterised by decreased lignin content as the use of plants for biofuel production requires the removal of lignin, and for forage lignin affects the digestibility of forage crops. In one embodiment, the plant is maize.

In a further embodiment, the laccase gene is selected from laccase 3 and laccase 5, wherein the laccase 3 gene encodes a polypeptide as defined in SEQ ID NO: 1 or a functional variant thereof and wherein the laccase gene 5 encodes a polypeptide as defined in SEQ ID NO: 4 or a functional variant thereof. More preferably the laccase 3 gene comprises a nucleic acid sequence as defined in SEQ ID NO: 2 or 3 or a functional variant thereof. Similarly, in a preferred embodiment, the laccase 5 gene comprises a nucleic acid sequence as defined in SEQ ID NO: 5 or 6 or a functional variant thereof.

In one embodiment, the method comprises introducing at least one mutation into at least one laccase gene and/or promoter wherein the mutation decreases or abolishes the expression or activity of the laccase nucleic acid compared to a wild type control. In one embodiment, the expression or activity is decreased by up to or more than 5%, 10%, 20%, 30%, 40%, 50%, 60%, 70%, 80% or 90% when compared to the level in a wild-type or control plant. By “abolished” is menat that no LAC3 and/or LAC5 is expressed or can be detectably expressed. In preferred embodiments, such mutations are introduced using targeted genome modification, preferably ZFNs, TALENs or CRISPR/Cas9, as described above. In alternative embodiments, such mutations are introduced using mutagenesis, preferably TILLING or T-DNA insertion, as also described above. In such embodiments the invention relates to a method and plant that has been generated by genetic engineering methods as described above, and does not encompass naturally occurring varieties.

In an alternative embodiment, the method comprises using RNA interference to reduce or abolish the expression of at least one laccase nucleic acid, preferably LAC3 and/or 5 nucleic acid. For example, expression of a laccase nucleic acid, as defined herein, can be reduced or silenced using a number of gene silencing methods known to the skilled person, such as, but not limited to, the use of small interfering nucleic acids (siNA) against LAC3 and/or 5. “Gene silencing” is a term generally used to refer to suppression of expression of a gene via sequence-specific interactions that are mediated by RNA molecules. The degree of reduction may be so as to totally abolish production of the encoded gene product, but more usually the abolition of expression is partial, with some degree of expression remaining. The term should not therefore be taken to require complete “silencing” of expression.

In one embodiment, the siNA may include, short interfering RNA (siRNA), double-stranded RNA (dsRNA), micro-RNA (miRNA), antagomirs and short hairpin RNA (shRNA) capable of mediating RNA interference.

The inhibition of expression and/or activity can be measured by determining the presence and/or amount of laccase 3 and/or 5 transcript using techniques well known to the skilled person (such as Northern Blotting, RT-PCR and so on).

Transgenes may be used to suppress endogenous plant genes. This was discovered originally when chalcone synthase transgenes in petunia caused suppression of the endogenous chalcone synthase genes and indicated by easily visible pigmentation changes. Subsequently it has been described how many, if not all plant genes can be “silenced” by transgenes. Gene silencing requires sequence similarity between the transgene and the gene that becomes silenced. This sequence homology may involve promoter regions or coding regions of the silenced target gene. When coding regions are involved, the transgene able to cause gene silencing may have been constructed with a promoter that would transcribe either the sense or the antisense orientation of the coding sequence RNA. It is likely that the various examples of gene silencing involve different mechanisms that are not well understood. In different examples there may be transcriptional or post-transcriptional gene silencing and both may be used according to the methods of the invention.

The mechanisms of gene silencing and their application in genetic engineering, which were first discovered in plants in the early 1990s and then shown in Caenorhabditis elegans are extensively described in the literature.

RNA-mediated gene suppression or RNA silencing according to the methods of the invention includes co-suppression wherein over-expression of the target sense RNA or mRNA, that is the laccase 3 and/or 5 sense RNA or mRNA, leads to a reduction in the level of expression of the genes concerned. RNAs of the transgene and homologous endogenous gene are co-ordinately suppressed. Other techniques used in the methods of the invention include antisense RNA to reduce transcript levels of the endogenous target gene in a plant. In this method, RNA silencing does not affect the transcription of a gene locus, but only causes sequence-specific degradation of target mRNAs. An “antisense” nucleic acid sequence comprises a nucleotide sequence that is complementary to a “sense” nucleic acid sequence encoding a laccase protein, or a part of the protein, i.e. complementary to the coding strand of a double-stranded cDNA molecule or complementary to an mRNA transcript sequence. The antisense nucleic acid sequence is preferably complementary to the endogenous laccase gene to be silenced. The complementarity may be located in the “coding region” and/or in the “non-coding region” of a gene. The term “coding region” refers to a region of the nucleotide sequence comprising codons that are translated into amino acid residues. The term “non-coding region” refers to 5′ and 3′ sequences that flank the coding region that are transcribed but not translated into amino acids (also referred to as 5′ and 3′ untranslated regions).

Antisense nucleic acid sequences can be designed according to the rules of Watson and Crick base pairing. The antisense nucleic acid sequence may be complementary to the entire laccase 3 or 5 nucleic acid sequence as defined herein, but may also be an oligonucleotide that is antisense to only a part of the nucleic acid sequence (including the mRNA 5′ and 3′ UTR). For example, the antisense oligonucleotide sequence may be complementary to the region surrounding the translation start site of an mRNA transcript encoding a polypeptide. The length of a suitable antisense oligonucleotide sequence is known in the art and may start from about 50, 45, 40, 35, 30, 25, 20, 15 or 10 nucleotides in length or less. An antisense nucleic acid sequence according to the invention may be constructed using chemical synthesis and enzymatic ligation reactions using methods known in the art. For example, an antisense nucleic acid sequence (e.g., an antisense oligonucleotide sequence) may be chemically synthesized using naturally occurring nucleotides or variously modified nucleotides designed to increase the biological stability of the molecules or to increase the physical stability of the duplex formed between the antisense and sense nucleic acid sequences, e.g., phosphorothioate derivatives and acridine-substituted nucleotides may be used. Examples of modified nucleotides that may be used to generate the antisense nucleic acid sequences are well known in the art. The antisense nucleic acid sequence can be produced biologically using an expression vector into which a nucleic acid sequence has been subcloned in an antisense orientation (i.e., RNA transcribed from the inserted nucleic acid will be of an antisense orientation to a target nucleic acid of interest). Preferably, production of antisense nucleic acid sequences in plants occurs by means of a stably integrated nucleic acid construct comprising a promoter, an operably linked antisense oligonucleotide, and a terminator.

The nucleic acid molecules used for silencing in the methods of the invention hybridize with or bind to mRNA transcripts and/or insert into genomic DNA encoding a polypeptide to thereby inhibit expression of the protein, e.g., by inhibiting transcription and/or translation. The hybridization can be by conventional nucleotide complementarity to form a stable duplex, or, for example, in the case of an antisense nucleic acid sequence which binds to DNA duplexes, through specific interactions in the major groove of the double helix. Antisense nucleic acid sequences may be introduced into a plant by transformation or direct injection at a specific tissue site. Alternatively, antisense nucleic acid sequences can be modified to target selected cells and then administered systemically. For example, for systemic administration, antisense nucleic acid sequences can be modified such that they specifically bind to receptors or antigens expressed on a selected cell surface, e.g., by linking the antisense nucleic acid sequence to peptides or antibodies which bind to cell surface receptors or antigens. The antisense nucleic acid sequences can also be delivered to cells using vectors.

RNA interference (RNAi) is another post-transcriptional gene-silencing phenomenon which may be used according to the methods of the invention. This is induced by double-stranded RNA in which mRNA that is homologous to the dsRNA is specifically degraded. It refers to the process of sequence-specific post-transcriptional gene silencing mediated by short interfering RNAs (siRNA). The process of RNAi begins when the enzyme, DICER, encounters dsRNA and chops it into pieces called small-interfering RNAs (siRNA). This enzyme belongs to the RNase III nuclease family. A complex of proteins gathers up these RNA remains and uses their code as a guide to search out and destroy any RNAs in the cell with a matching sequence, such as target mRNA.

Artificial and/or natural microRNAs (miRNAs) may be used to knock out gene expression and/or mRNA translation. MicroRNAs (miRNAs) miRNAs are typically single stranded small RNAs typically 19-24 nucleotides long. Most plant miRNAs have perfect or near-perfect complementarity with their target sequences. However, there are natural targets with up to five mismatches. They are processed from longer non-coding RNAs with characteristic fold-back structures by double-strand specific RNases of the Dicer family. Upon processing, they are incorporated in the RNA-induced silencing complex (RISC) by binding to its main component, an Argonaute protein. miRNAs serve as the specificity components of RISC, since they base-pair to target nucleic acids, mostly mRNAs, in the cytoplasm. Subsequent regulatory events include target mRNA cleavage and destruction and/or translational inhibition. Effects of miRNA overexpression are thus often reflected in decreased mRNA levels of target genes. Artificial microRNA (amiRNA) technology has been applied in Arabidopsis thaliana and other plants to efficiently silence target genes of interest. The design principles for amiRNAs have been generalized and integrated into a Web-based tool (http://wmd.weiqelworld.orq).

Thus, according to the various aspects of the invention a plant may be transformed to introduce a RNAi, shRNA, snRNA, dsRNA, siRNA, miRNA, ta-siRNA, amiRNA or cosuppression molecule that has been designed to target the expression of an laccase nucleic acid sequence and selectively decreases or inhibits the expression of the gene or stability of its transcript. Preferably, the RNAi, snRNA, dsRNA, shRNA siRNA, miRNA, amiRNA, ta-siRNA or cosuppression molecule used according to the various aspects of the invention comprises a fragment of at least 17 nt, preferably 22 to 26 nt and can be designed on the basis of the information shown in any of SEQ ID Nos. 2, 3, 5 or 6. Guidelines for designing effective siRNAs are known to the skilled person. Briefly, a short fragment of the target gene sequence (e.g., 19-40 nucleotides in length) is chosen as the target sequence of the siRNA of the invention. The short fragment of target gene sequence is a fragment of the target gene mRNA. In preferred embodiments, the criteria for choosing a sequence fragment from the target gene mRNA to be a candidate siRNA molecule include 1) a sequence from the target gene mRNA that is at least 50-100 nucleotides from the 5′ or 3′ end of the native mRNA molecule, 2) a sequence from the target gene mRNA that has a G/C content of between 30% and 70%, most preferably around 50%, 3) a sequence from the target gene mRNA that does not contain repetitive sequences (e.g., AAA, CCC, GGG, TTT, AAAA, CCCC, GGGG, TTTT), 4) a sequence from the target gene mRNA that is accessible in the mRNA, 5) a sequence from the target gene mRNA that is unique to the target gene, 6) avoids regions within 75 bases of a start codon. The sequence fragment from the target gene mRNA may meet one or more of the criteria identified above. The selected gene is introduced as a nucleotide sequence in a prediction program that takes into account all the variables described above for the design of optimal oligonucleotides. This program scans any mRNA nucleotide sequence for regions susceptible to be targeted by siRNAs. The output of this analysis is a score of possible siRNA oligonucleotides. The highest scores are used to design double stranded RNA oligonucleotides that are typically made by chemical synthesis. In addition to siRNA which is complementary to the mRNA target region, degenerate siRNA sequences may be used to target homologous regions. siRNAs according to the invention can be synthesized by any method known in the art. RNAs are preferably chemically synthesized using appropriately protected ribonucleoside phosphoramidites and a conventional DNA/RNA synthesizer. Additionally, siRNAs can be obtained from commercial RNA oligonucleotide synthesis suppliers.

siRNA molecules according to the aspects of the invention may be double stranded. In one embodiment, double stranded siRNA molecules comprise blunt ends. In another embodiment, double stranded siRNA molecules comprise overhanging nucleotides (e.g., 1-5 nucleotide overhangs, preferably 2 nucleotide overhangs). In some embodiments, the siRNA is a short hairpin RNA (shRNA); and the two strands of the siRNA molecule may be connected by a linker region (e.g., a nucleotide linker or a non-nucleotide linker). The siRNAs of the invention may contain one or more modified nucleotides and/or non-phosphodiester linkages. Chemical modifications well known in the art are capable of increasing stability, availability, and/or cell uptake of the siRNA. The skilled person will be aware of other types of chemical modification which may be incorporated into RNA molecules.

In another alternative embodiment, the method comprises introducing and expressing a nucleic acid construct comprising at least one nucleic acid wherein the nucleic acid encodes a miRNA as defined in SEQ ID NO: 10 or a functional variant thereof operably linked to a regulatory sequence. Preferably, the nucleic acid construct comprises at least one nucleic acid sequence, wherein the nucleic acid sequence is selected from SEQ ID NO: 9, 32 or 39. In another embodiment, the nucleic acid construct comprises two nucleic acid sequences selected from SEQ ID NO: 32 and 39. Preferably the or each nucleic acid sequence(s) are operably linked to a regulatory sequence, such as a promoter. Examples of suitable promoter sequences would be known to the skilled person but are also described above.

In a further alternative embodiment, the method comprises introducing an miR528 comprising SEQ ID NO: 9 or a functional variant thereof.

In another aspect of the invention, there is provided a genetically altered plant, part thereof or plant cell, wherein said plant is characterised by altered expression or levels of at least one laccase gene and/or altered expression or activity of miR528. Preferably, the plant can also be characterised by an altered lignin content. Again, as described above an “alteration” may be considered to be an increase or decrease.

In one embodiment, the plant is characterised by an increased expression of at least one laccase gene and/or decreased expression or activity of miR528 compared to a wild-type or control plant, as described above.

In one embodiment, the plant expresses a polynucleotide “exogenous” or “endogenous” to an individual plant that is a polynucleotide, which is introduced into the plant by any means other than by a sexual cross. Examples of means by which this can be accomplished are described below. In one embodiment, an exogenous nucleic acid is expressed in the plant which is a nucleic acid construct comprising a nucleic acid wherein the nucleic acid encodes a laccase 3 polypeptide as defined in SEQ ID NO: 1 or a functional variant or homologue thereof and/or a nucleic acid encoding a laccase 5 polypeptide as defined in SEQ ID NO: 4 or a functional variant or homologue thereof, wherein both laccase sequences are operably linked to a regulatory sequence.

Preferably, the nucleic acid encoding laccase 3 comprises a sequence as defined in SEQ ID NO: 2 or 3 or a functional variant or homologue thereof and preferably the nucleic acid encoding laccase 5 comprises a sequence as defined in SEQ ID NO: 5 or 6 or a functional variant or homologue thereof.

In another embodiment, the plant expresses a miR528 inhibitor. Specifically, in one example the plant may express a nucleic acid construct comprising a nucleic acid sequence as defined in SEQ ID NO: 7 or 16 or a functional variant thereof operably linked to a regulatory sequence. A regulatory sequence is described above. In another example, the miR528 inhibitor is an RNA molecule comprising an RNA sequence as defined in SEQ ID NO: 8 or a functional variant thereof.

In an alternative embodiment, the plant comprises at least one mutation in at least one nucleic acid encoding a laccase nucleic acid, preferably wherein the laccase nucleic acid is selected from laccase 3 and 5, and wherein the mutation is in a miR528 binding site. In one embodiment a mutation is introduced into both the LAC3 and LAC5 miR528 binding site. The mutation may be introduced using any of the mutagenesis methods described above.

The miR528 binding site in LAC3 is as follows:

(SEQ ID NO: 20)
CUCUGCUGCAUGCCCCUUCGA

The miR528 binding site in LAC5 is as follows:

(SEQ ID NO: 21)
CUUCUCCGCAUGUCCCUUCCU

Preferably, the mutation is any mutation that prevents the binding of miR528 to LAC3 and/or LAC5. In one example, the mutation may be selected from a deletion, insertion and substitution of one or more nucleotides in SEQ ID NO: 20 and/or 21 described above. In one example, 1, 2, 3, 4, 5, 6, 7, 8, 9, 10, 11, 12, 13, 14, 15, 16, 17, 18, 19 or 20 nucleotides are deleted or inserted.

In a further alternative embodiment, the plant comprises at least one mutation in a miR528a and/or b gene or in the miR528 promoter such that the expression of miR528 is reduced or abolished. Again, the mutation may be introduced using any of the mutagenesis methods described above.

In another aspect of the invention, there is provided a genetically altered plant, wherein the plant is characterised by decreased expression of at least one laccase gene and/or increased expression or activity of miR528 compared to a wild-type or control plant.

In another aspect of the invention, there is provided a method of producing a plant with altered resistance to lodging in a plant, the method comprising altering the expression or levels of at least one laccase gene and/or altering the expression or activity of miR528. Preferably, the plant also has an altered lignin content compared to a wild-type or control plant.

In a preferred embodiment, the plant has increased resistance to lodging in a plant, and the method comprises increasing the expression of at least one laccase gene and/or decreasing the expression or activity of miR528. Preferably, increasing the expression of at least one laccase gene comprises introducing and expressing a nucleic acid construct comprising at least one nucleic acid wherein the nucleic acid encodes a laccase 3 polypeptide as defined in SEQ ID NO: 1 or a functional variant or homologue thereof and/or a laccase 5 polypeptide as defined in SEQ ID NO: 4 or a functional variant or homologue thereof operably linked to a regulatory sequence.

In an alternative embodiment, decreasing the activity of miR528 comprises introducing and expressing a nucleic acid construct comprising a miR528 inhibitor or expressing a miR528 inhibitor in the plant. In a preferred embodiment, the nucleic acid construct comprises a nucleic acid sequence encoding a miR528 inhibitor, where the sequence of the miR528 inhibitor is defined in SEQ ID NO: 7 or 16 or a functional variant thereof operably linked to a regulatory sequence. A regulatory sequence is described above. In another preferred embodiment, the miR528 inhibitor is an RNA molecule comprising an RNA sequence as defined in SEQ ID NO: 8 or a functional variant thereof.

Transformation methods for generating a transgenic plant of the invention are known in the art. Thus, according to the various aspects of the invention, any nucleic acid construct described herein is introduced into a plant and expressed as a transgene. The nucleic acid construct is introduced into said plant through a process called transformation. The terms “introduction” or “transformation” as referred to herein encompass the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

Transformation of plants is now a routine technique in many species. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, and transformation using viruses or pollen and microinjection. Methods may be selected from the calcium/polyethylene glycol method for protoplasts, electroporation of protoplasts, microinjection into plant material, DNA or RNA-coated particle bombardment, infection with (non-integrative) viruses and the like. Transgenic plants, including transgenic crop plants, are preferably produced via Agrobacterium tumefaciens mediated transformation.

To select transformed plants, the plant material obtained in the transformation is subjected to selective conditions so that transformed plants can be distinguished from untransformed plants. For example, the seeds obtained in the above-described manner can be planted and, after an initial growing period, subjected to a suitable selection by spraying. A further possibility is growing the seeds, if appropriate after sterilization, on agar plates using a suitable selection agent so that only the transformed seeds can grow into plants. Alternatively, the transformed plants are screened for the presence of a selectable marker. Following DNA transfer and regeneration, putatively transformed plants may also be evaluated, for instance using Southern blot analysis, for the presence of the gene of interest, copy number and/or genomic organisation. Alternatively or additionally, expression levels of the newly introduced DNA may be monitored using Northern and/or Western blot analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The method may further comprise regenerating a transgenic plant from the plant or plant cell wherein the transgenic plant comprises in its genome a nucleic acid sequence selected from SEQ ID NO: 2, 3, 5, 6 and 7 or a nucleic acid that encodes a laccase protein as defined in SEQ ID NO: 1 or 4 and obtaining a progeny plant derived from the transgenic plant, wherein said progeny exhibits an increase in at least one of lodging resistance, lignin content and yield.

In another aspect of the invention there is provided a method for producing a genetically altered plant as described herein. In one embodiment, the method comprises introducing at least one mutation into the miR528 binding site of at least one LAC3 and/or LAC5 nucleic acid, as described above, of preferably at least one plant cell using any mutagenesis technique described herein. Preferably said method further comprises regenerating a plant from the mutated plant cell.

Accordingly, in one embodiment, the method comprises
a. selecting a part of the plant;
b. transfecting at least one cell of the part of the plant of paragraph (a) with at least one nucleic acid construct or miR528 inhibitor as described herein or
c. regenerating at least one plant derived from the transfected cell or cells;
d. selecting one or more plants obtained according to paragraph (c) that show increased expression of LAC3 and/or LAC5 or reduced miR528 activity.

The method may further comprise selecting one or more mutated plant cells or plants, preferably for further propagation. Preferably said selected plants comprise at least one mutation in the miR528 binding site of at least one LAC3 and/or LAC5 nucleic acid. In one embodiment, said plants are characterised by increased level of lodging resistance (or a decrease in lodging) or an increased level of lignin content, preferably in the stem and/or roots.

The selected plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

In a further embodiment of any of the methods described herein, the method may further comprise at least one or more of the steps of assessing the phenotype of the transgenic or genetically altered plant, measuring at least one of an increase in lodging resistance, lignin content, rind penetrometer resistance and cellulose/hemicellulose content, as described above. In other words, the method may involve the step of screening the plants for the desired phenotype.

In a further aspect of the invention there is provided a plant obtained or obtainable by the above described methods.

In a further final aspect of the invention, there is provided a method of screening a population of plants and identifying and/or selecting a plant that will has increased expression of laccase 3 and/or 5 and/or reduced expression/activity of miR528 and/or a mutation in the miR528 binding site of laccase 3 and/or 5. Such plants will have an increased lignin content and therefore an increased level of resistance to lodging compared to a wild type or control plant. As discussed above, such screening methods are of particular value as, unless the conditions for lodging occur in a growing season, it is difficult to for breeders, farmers, crop testers or the like to determine whether a variety is resistant to lodging. As such, being able to determine whether an individual plant or variety will be resistant to lodging before planting has the potential to have significant economic benefit.

In particular, the method may comprise detecting at least one polymorphism or mutation in a laccase 3 and/or 5 gene and/or promoter, where such mutation leads to an increased level of laccase 3 and/or 5 expression or where such mutation is in a miR528 binding site and as such, prevents the binding of miR528 to laccase 3 and/or 5. An increase in expression or a decrease in miR528 binding may be relative to that in a plant that does not carry the mutation. Such plants may be used to determine a threshold value to which the levels can be compared in plants to be screened. Alternatively, the method may comprise detecting at least one polymorphism or mutation in a miR528a and/or b gene and/or promoter thereof such that the miR528 expression is reduced or abolished or the miR528 is unable to bind the its target—laccase 3 and/or laccase 5. Again, the level of expression or activity may be relative to a plant that does not carry the mutation. The mutation may be at least one addition, substitution or deletion.

Suitable tests for assessing the presence of a polymorphism or mutation would be well known to the skilled person, and include but are not limited to, Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs-which are also referred to as Microsatellites), and Single Nucleotide Polymorphisms (SNPs). In one embodiment, Kompetitive Allele Specific PCR (KASP) genotyping is used.

In one embodiment, the method comprises

a) obtaining a nucleic acid sample from a plant and
b) carrying out nucleic acid amplification of one or more laccase 3 gene and/or promoter, laccase 5 gene and/or promoter or miR528a and/or b gene and/or promoter alleles using one or more primer pairs.

In a further embodiment, the method may further comprise introgressing the chromosomal region comprising at least one of said high-laccase 3 or laccase 5 or low miR528-expressing/activity polymorphisms into a second plant or plant germplasm to produce an introgressed plant or plant germplasm. Preferably the expression or activity of laccase 3 or laccase 5 will be increased or the expression or activity of miR528 in said second plant will be reduced or abolished (compared to a control or wild-type plant), and more preferably said second plant will display an alteration in lodging resistance as described above.

In a further aspect of the invention there is provided a method of altering, preferably increasing lodging resistance in a plant, the method comprising
a. screening a population of plants for at least one plant with at least one of the above described polymorphisms or mutations;
b. further increasing the expression of laccase 3 and/or 5 by any of the methods described herein or further reducing or abolishing the expression or activity of at least one miR528 nucleic by any of the methods described herein.

By “further increasing” or “further reducing” is meant increasing or reducing the level of expression or activity to a level higher or lower respectively than that in the plant with the at least one of the above-described polymorphisms. Such an increase or decrease in expression and/or activity can be up to 10%, 20%, 30%, 40%, 50%, 60%, 70%, 80% or 90% when compared to the level in a control plant.

A plant according to all aspects of the invention described herein may be a monocot or a dicot plant. Preferably, the plant is a crop plant or a granineous plant. By crop plant is meant any plant which is grown on a commercial scale for human or animal consumption or use. In a preferred embodiment, the plant is a cereal.

In a preferred embodiment, the plant is maize. In one example, the maize may be “maize variety zong 31” or “maize B73”. For “Maize Variety Zong 31”, reference is made to Yang Hui, Wang Guoying, Dai Jingrui, Research on the transformation of elite maize inbred lines Zong 3 and 31, Journal of Agricultural Biotechnology, 2001, No. 04. For “Maize B73”, reference is made to Gong Fuquan, Li Pinghua, Cloning of pyruvate phosphate dikinase gene in maize inbred line B73 and the effect of low nitrogen on the expression of PPDK, Journal of Tropical Biology, 2014, No. 4.

The term “plant” as used herein encompasses whole plants, ancestors and progeny of the plants and plant parts, including seeds, fruit, shoots, stems, leaves, roots (including tubers), flowers, tissues and organs, wherein each of the aforementioned comprise the nucleic acid construct as described herein or carry the herein described mutations. The term “plant” also encompasses plant cells, suspension cultures, callus tissue, embryos, meristematic regions, gametophytes, sporophytes, pollen and microspores, again wherein each of the aforementioned comprises the nucleic acid construct or mutations as described herein. In one example only the plant cell is a cell that is not capable of photosynthesis. For example, the plant cell may lack chloroplasts. The cell may also be from one of the following tissue types, including leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem).

The invention also extends to harvestable parts of a plant of the invention as described herein, but not limited to seeds, leaves, fruits, flowers, stems, roots, rhizomes, tubers and bulbs. The aspects of the invention also extend to products derived, preferably directly derived, from a harvestable part of such a plant, such as dry pellets or powders, oil, fat and fatty acids, starch or proteins. Another product that may be derived from the harvestable parts of the plant of the invention is biodiesel. The invention also relates to food products and food supplements comprising the plant of the invention or parts thereof. In one embodiment, the food products may be animal feed. In another aspect of the invention, there is provided a product derived from a plant as described herein or from a part thereof.

In a most preferred embodiment, the plant part or harvestable product is a seed or grain. Therefore, in a further aspect of the invention, there is provided a seed produced from a genetically altered plant as described herein.

In an alternative embodiment, the plant part is pollen, a propagule or progeny of the genetically altered plant described herein. Accordingly, in a further aspect of the invention there is provided pollen, a propagule or progeny produced from a genetically altered plant as described herein.

A control plant as used herein according to all of the aspects of the invention is a plant which has not been modified according to the methods of the invention. Accordingly, in one embodiment, the control plant does not have altered expression or levels of at least one laccase gene and/or altered the expression or activity of miR528 as described above. In an alternative embodiment, the plant has not been genetically modified, as described above. In one embodiment, the control plant is a wild type plant. The control plant is typically of the same plant species, preferably having the same genetic background as the modified plant.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

The foregoing application, and all documents and sequence accession numbers cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

The invention is now described in the following non-limiting examples.

### Example 1: Production and Identification of Overexpressing Plants

I. Construction of Recombinant Plasmid

1. Total RNA was extracted from maize B73, and subjected to reverse transcription to obtain the cDNA.

2. PCR amplification was performed by using the cDNA obtained in Step 1 as a template, and using a primer pair consisting of F1 and R1, to obtain a PCR amplification product. The PCR amplification product was sequenced to have a sequence as shown in SEQ ID NO: 12.

F1 (SEQ ID NO: 22):
5′-GACTCTAGAGGATCCATGCCCCTTCGACAACGTC-3′;
and
R1 (SEQ ID NO: 23):
5′-GGTACCCGGGGATCCTCAGCACTTGGGCATGTTAGG-3′.

3. A pCUB vector was enzymatically cleaved by the restriction endonuclease BamHI, to obtain a linearized vector.

4. The linearized vector obtained in Step 3 was subjected to In-Fusion based homologous recombination with the PCR amplification product obtained in Step 2, to obtain a recombinant plasmid pCUB-ZmMZS. After sequencing, the recombinant plasmid pCUB-ZmMZS was found to have a structure in which a DNA molecule having a sequence as shown in SEQ ID NO: 3 was inserted in the BamHI cleavage site of the pCUB vector.

II. Production of Transgenic Plants The maize variety Zong 31 was used as a starting plant. The maize variety Zong 31 was also known as wild-type plant, and was indicated by WT in the figures.

The recombinant plasmid pCUB-ZmMZS was transformed into the starting plant, to obtain a transgenic plant of generation T₀. The transgenic plant of generation T₀ was inbred to obtain a plant of generation T₁. The plant of generation T₁ was further inbred to obtain a plant of generation T₂. The homozygous transgenic line obtained at the generation T₂ was designated as ZmMZS gene-over expressing line. Two lines over expressing the ZmMZS gene (lines #3 and #4) were randomly selected and used in subsequent tests.

A pCUB vector was transformed into the starting plant, to obtain a transgenic plant of generation T₀. The transgenic plant of generation T₀ was inbred to obtain a plant of generation T₁. The plant of generation T₁ was further inbred to obtain a plant of generation T₂. The homozygous transgenic line obtained at the generation T₂ was designated as empty vector transformed line.

II. Detection by Real-Time Fluorescence Quantitative PCR

The test plants were plants of generation T₂ of line #3, plants of generation T₂ of line #4, and the starting plant.

3 plants of each line were used and the results were averaged.

The total RNA was extracted from the leaves of the test plants grown for 7 days (from the day of germination) under hydroponic culture conditions, and PCR amplification was performed by using a primer pair consisting of F2 and R2, to identify the relative expression level of the ZmMZS gene. The Ubi gene was used as an internal reference gene (where the primer pair for identifying the internal reference gene consisted of F3 and R3).

F2 (SEQ ID NO: 24):
5′-GCGTGTTGTTGTTAGCATTTGG-3′;
R2 (SEQ ID NO: 25):
5′-GGGTGATGTTCTTGTAGCCCTG-3′.
F3 (SEQ ID NO: 26):
5′-GCTGCCGATGTGCCTGCGTCG-3′;
R3 (SEQ ID NO: 27):
5′-CTGAAAGACAGAACATAATGAGCACAG-3′.

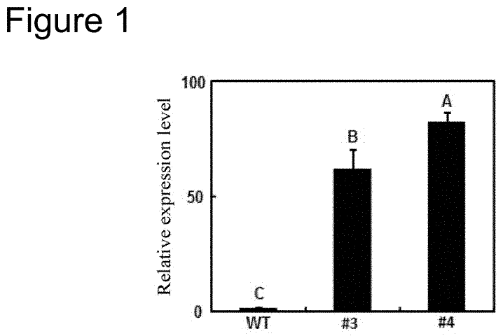

The results are shown in FIG. 1. Compared with the wild-type plants, the relative expression levels of the ZmMZS gene in the plants of lines #3 and #4 are increased significantly.

IV. Histochemical Staining

The test plants were plants of generation T₂ of line #3, plants of generation T₂ of line #4, plants of generation T₂ of empty vector-transformed line, and the starting plant.

The first internodes, the leaves and the maturation region of the root of the test plants grown for 30 days (from the day of germination) under hydroponic culture conditions were sliced (where the thickness of the slice was 50 μm), stained with 5% phloroglucin for 2 min, added with 1 drop of hydrochloric acid, and observed under a microscope.

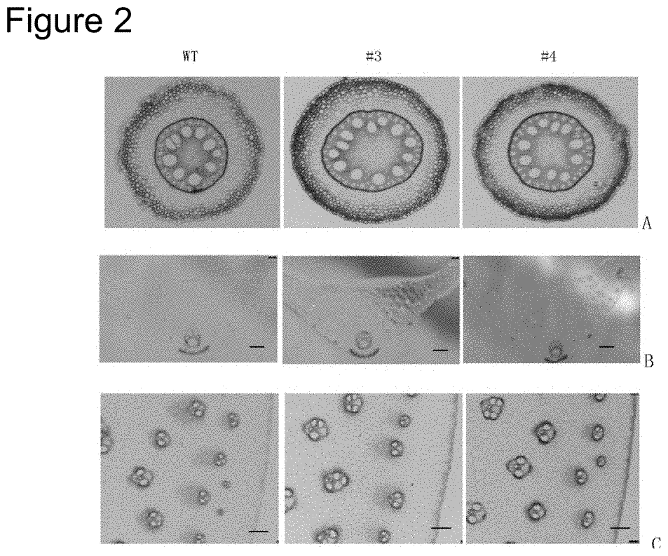

The microphotographs of the root are shown in FIG. 2A (in which the scale bar is 75 μm).

The microphotographs of the leaves are shown in FIG. 2B (in which the scale bar is 75 μm).

The microphotographs of the first internodes are shown in FIG. 2C (in which the scale bar is 75 μm).

The staining intensity in both the aboveground parts and the roots of the plants of lines #3 and #4 is darker (i.e., the lignin content is increased) compared to wild-type plants. The staining intensity in both the aboveground parts and the roots of the plants of the empty vector transformed line is consistent with that in the wild-type plants.

V. Determination of Lignin Content

The test plants were plants of generation T₂ of line #3, plants of generation T₂ of line #4, plants of generation T₂ of empty vector-transformed line, and the starting plant. 3 plants of each line were used and the results were averaged.

At day 7 after pollination in the maturation period, the third internode of the test plants was dried at 80° C. to a constant weight, then crushed and sieved through a 40-mesh sieve to collect the powder. About 5 mg of the powder was weighed into a 10 ml glass test tube and the lignin content was measured using a lignin content assay kit available from Suzhou Keming Science and Technology Co., Ltd (acetyl bromide method, see the instruction for specific procedures).

Lignin content (in mg/g, where mg is the mass unit of lignin, g is the mass unit of the powder on dry basis)=0.0735*(ΔA-0.0068)/W*T where A: absorbance at 280 nm; ΔA=A of the test tube-A of the blank tube; W: mass of the sample in g; and T: dilution factor.

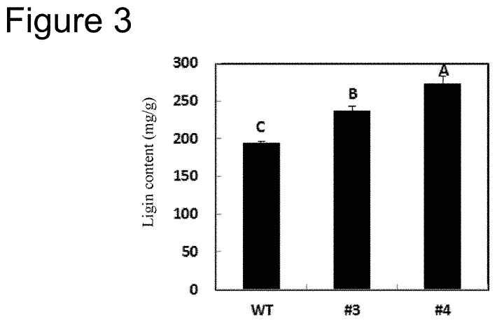

The results are shown in FIG. 3 Compared with the wild-type plants, the lignin content is increased by 21.62% in the stems of the plant of line #3, and by 40.10% in the stems of the plant of line #4. Compared with the wild-type plants, there is no significant change in the lignin content in the stems of the plant of empty vector transformed line.

### Example 2: Production and Identification of Underexpressing Plants

I. Construction of Recombinant Plasmid

A double-stranded DNA molecule having a sequence as shown in SEQ ID NO: 13 was inserted into the BamHI cleavage site of the pCUB vector, to obtain a recombinant plasmid pCUB-ZmMZS-Ri. The double-stranded DNA molecule having a sequence as shown in SEQ ID NO: 13 expresses a RNA molecule having a sequence as shown in SEQ ID NO: 14. The RNA molecule having a sequence as shown in SEQ ID NO: 14 is a precursor RNA of the RNA having a sequence as shown in SEQ ID NO: 15. The RNA having a sequence as shown in SEQ ID NO: 15 is a miRNA targeting the ZmMZS gene.

II. Production of Transgenic Plants

The maize variety Zong 31 was used as a starting plant. The maize variety Zong 31 was also known as wild-type plant, and was indicated by WT in the figures.

The recombinant plasmid pCUB-ZmMZS-Ri was transformed into the starting plant, to obtain a transgenic plant of generation T₀. The transgenic plant of generation T₀ was inbred to obtain a plant of generation T₁. The plant of generation T₁ was further inbred to obtain a plant of generation T₂. The homozygous transgenic line obtained at the generation T₂ was designated as ZmMZS gene-underexpressing line. A ZmMZS gene-underexpressing line (line #5) was randomly selected and used in subsequent tests.

A pCUB vector was transformed into the starting plant, to obtain a transgenic plant of generation T₀. The transgenic plant of generation T₀ was inbred to obtain a plant of generation T₁. The plant of generation T₁ was further inbred to obtain a plant of generation T₂. The homozygous transgenic line obtained at the generation T₂ was designated as empty vector transformed line.

III. Histochemical Staining

The test plants were plants of generation T₂ of line #5, plants of generation T₂ of empty vector-transformed line, and the starting plant.

The first internodes, and the maturation region of the root of the test plants grown for 30 days (from the day of germination) under hydroponic culture conditions were sliced (where the thickness of the slice was 50 μm), stained with 5% phloroglucin for 2 min, added with 1 drop of hydrochloric acid, and observed under a microscope.

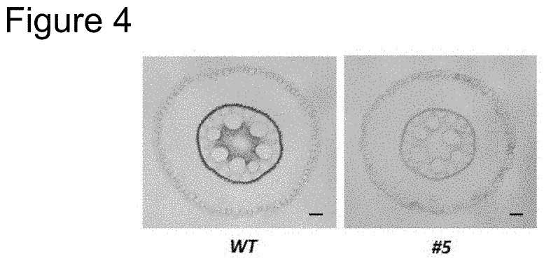

The microphotographs of the root are shown in FIG. 4 (in which the scale bar is 75 μm).

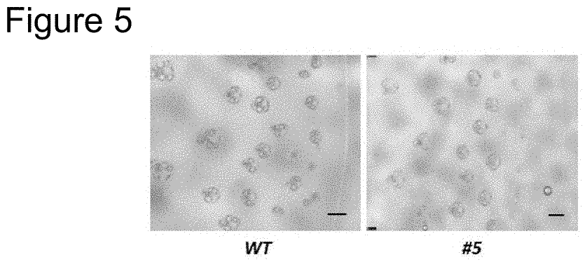

The microphotographs of the first internodes are shown in FIG. 5 (in which the scale bar is 200 μm).

The staining intensity in both the aboveground parts and the roots of the plants of line #5 becomes light (i.e., the lignin content is reduced) compared to wild-type plants. The staining intensity in both the aboveground parts and the roots of the plants of the empty vector transformed line is consistent with that in the wild-type plants.

IV. Determination of Lignin Content

The test plants were plants of generation T₂ of line #5, plants of generation T₂ of empty vector-transformed line, and the starting plant.

3 plants of each line were used and the results were averaged.

At day 7 after pollination in the maturation period, the third internode of the test plants was dried at 80° C. to a constant weight, then crushed and sieved through a 40-mesh sieve to collect the powder. About 5 mg of the powder was weighed into a 10 ml glass test tube and the lignin content was measured using a lignin content assay kit available from Suzhou Keming Science and Technology Co., Ltd (acetyl bromide method, see the instruction for specific procedures).

Lignin content (in mg/g, where mg is the mass unit of lignin, g is the mass unit of the powder on dry basis)=0.0735*(ΔA-0.0068)/W*T where A: absorbance at 280 nm; ΔA=A of the test tube-A of the blank tube; W: mass of the sample in g; and T: dilution factor.

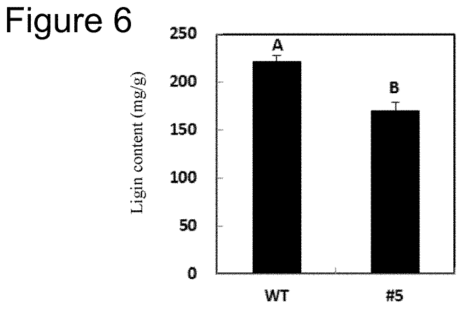

The results are shown in FIG. 6. Compared with the wild-type plants, the lignin content in the stems of the plant of line #5 is reduced by 22.93%. Compared with the wild-type plants, there is no significant change in the lignin content in the stems of the plant of empty vector transformed line.

### Example 3: Construction of Recombinant Plasmid

The pCUB vector is a circular plasmid having a sequence as shown in SEQ ID NO: 11.

I. A double-stranded DNA molecule having a sequence as shown in SEQ ID NO: 13 was inserted into the BamHI cleavage site of the pCUB vector, to obtain a recombinant plasmid pCUB-MIR. The recombinant plasmid pCUB-MIR was a plasmid over expressing a miRNA having a sequence as shown in SEQ ID NO: 15. The double-stranded DNA molecule having a sequence as shown in SEQ ID NO: 13 expresses a RNA molecule having a sequence as shown in SEQ ID NO: 14. An RNA molecule having a sequence as shown in SEQ ID NO: 14 was a precursor RNA of the miRNA having a sequence as shown in SEQ ID NO: 15.

II. A double-stranded DNA molecule having a sequence as shown in SEQ ID NO: 7 was inserted into the BamHI cleavage site of the pCUB vector, to obtain a recombinant plasmid pCUB-MIM. The portion other than “CTA” in the nucleotide sequence from positions 218-241 as shown in SEQ ID NO: 7 is reversely complementary to the DNA corresponding to the RNA having a sequence as shown in SEQ ID NO: 15. The recombinant plasmid pCUB-MIM was a plasmid inhibiting the expression of the miRNA having a sequence as shown in SEQ ID NO: 15.

### Example 4: Production and Phenotype Identification of Transgenic Plants

I. Production of Transgenic Plants

The maize variety Zong 31 was used as a starting plant. The maize variety Zong 31 was also known as wild-type plant, and was indicated by WT in the figures.

The recombinant plasmid pCUB-MIR was transformed into the starting plant, to obtain a transgenic plant of generation T₀. The transgenic plant of generation T₀ was inbred to obtain a plant of generation T₁. The plant of generation T₁ was further inbred to obtain a plant of generation T₂. The homozygous transgenic line obtained at the generation T₂ was designated as overexpressing line. An overexpressing (OE) line was selected at random and used in subsequent tests.

The recombinant plasmid pCUB-MIM was transformed into the starting plant, to obtain a transgenic plant of generation T₀. The transgenic plant of generation T₀ was inbred to obtain a plant of generation T₁. The plant of generation T₁ was further inbred to obtain a plant of generation T₂. The homozygous transgenic line obtained at the generation T₂ was designated as expression inhibited line. Two expression inhibited lines (TM-3 and TM-7) were selected at random, and used in subsequent tests.

A pCUB vector was transformed into the starting plant, to obtain a transgenic plant of generation T₀. The transgenic plant of generation T₀ was inbred to obtain a plant of generation T₁. The plant of generation T₁ was further inbred to obtain a plant of generation T₂. The homozygous transgenic line obtained at the generation T₂ was designated as empty vector transformed line.

II. Identification of miRNA Expression Level

The test plants were plants of generation T₂ of line OE, plants of generation T₂ of line TM-3, plants of generation T₂ of line TM-7, and the starting plant. 5 plants of each line were used and the results were averaged.

Total RNA was extracted from the leaves of the test plants, reverse transcription was performed using a RT primer, and then real-time quantitative PCR was performed using a primer pair consisting of F1 and R1, to detect the relative expression level of the miRNA having a sequence as shown in SEQ ID NO: 15 or 9. The Ubi gene was used as an internal reference gene (where the primer pair for identifying the internal reference gene consisted of F2 and R2).

(SEQ ID NO: 28) RT:
GTCGTATCCAGTGCAGGGTCCGAGGTATTCGCACTGGATACGACCTCCTC.
(SEQ ID NO: 29) F1:
5′-GTGGAAGGGGCATGCA-3′;
(SEQ ID NO: 30) R1:
5′-GTGCAGGGTCCGAGGT-3′.
(SEQ ID NO: 26) F2:
5′-GCTGCCGATGTGCCTGCGTCG-3′;
(SEQ ID NO: 27) R2:
5′-CTGAAAGACAGAACATAATGAGCACAG-3′.

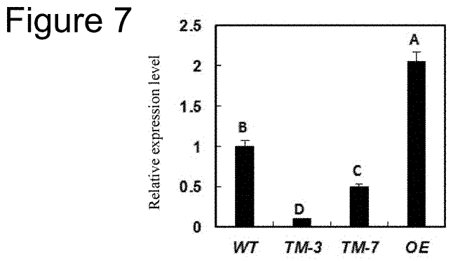

The results are shown in FIG. 7. Compared with the wild-type plants, the expression level of the miRNA having a sequence as shown in SEQ ID NO: 15 is significantly increased in the plants of line OE, and significantly reduced in the plants of lines TM-3 and the TM-7.

III. Histochemical Staining

The test plants were plants of generation T₂ of line OE, plants of generation T₂ of line TM-3, plants of generation T₂ of line TM-7, plants of generation T₂ of empty plasmid transformed line, and the starting plant.

The first internodes, and the maturation region of the root of the test plants grown for 30 days (from the day of germination) under hydroponic culture conditions were sliced (where the thickness of the slice was 50 μm), stained with 5% phloroglucin for 2 min, added with 1 drop of hydrochloric acid, and observed under a microscope.

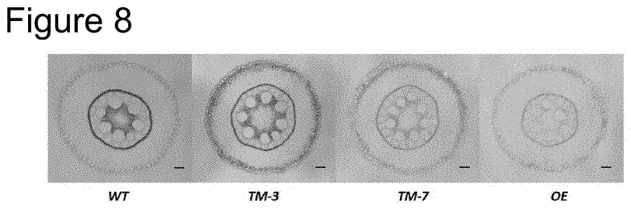

The microphotographs of the root are shown in FIG. 8 (in which the scale bar is 75 μm).

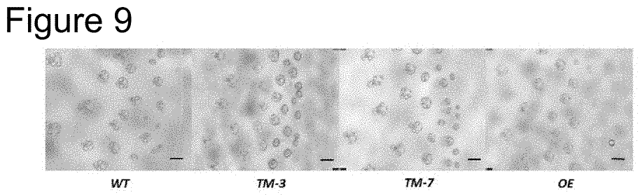

The microphotographs of the first internodes are shown in FIG. 9 (in which the scale bar is 200 μm).

The staining intensity becomes lighter (i.e., the lignin content is reduced) in the plants of line OE and becomes darker (i.e., the lignin content is increased) in the plants of lines TM-3 and TM-7 in both the aboveground parts and the roots compared to the wild-type plants. The staining intensity in both the aboveground parts and the roots of the plants of the empty vector transformed line is consistent with that in the wild-type plants.

IV. Determination of Lignin Content

The test plants were plants of generation T₂ of line OE, plants of generation T₂ of line TM-3, plants of generation T₂ of line TM-7, plants of generation T₂ of empty plasmid transformed line, and the starting plant. 3 plants of each line were used and the results were averaged.

At day 7 after pollination in the maturation period, the third internode of the test plants was dried at 80° C. to a constant weight, then crushed and sieved through a 40-mesh sieve to collect the powder. About 5 mg of the powder was weighed into a 10 ml glass test tube and the lignin content was measured using a lignin content assay kit available from Suzhou Keming Science and Technology Co., Ltd (acetyl bromide method, see the instruction for specific procedures).

Lignin content (in mg/g, where mg is the mass unit of lignin, g is the mass unit of the powder on dry basis)=0.0735*(ΔA-0.0068)/W*T where A: absorbance at 280 nm; ΔA=A of the test tube-A of the blank tube; W: mass of the sample in g; and T: dilution factor.

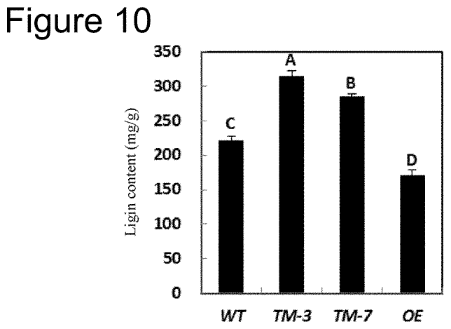

The results are shown in FIG. 10. Compared with the wild-type plants, the lignin content is reduced by 21.62% in the stems of the plant of line OE, and increased by 42.15% in the stems of the plant of line TM-3 and by 28.96% in the stems of the plant of line TM-7. Compared with the wild-type plants, there is no significant change in the lignin content in the stems of the plant of empty vector transformed line.

V. Determination of Puncture Strength of the Stem

The puncture strength of the stem is obviously correlated with the lodging resistance of the corn, and able to reflect the strength and the lodging resistance of the stem comprehensively.

The test plants were plants of generation T₂ of line OE, plants of generation T₂ of line TM-3, plants of generation T₂ of line TM-7, plants of generation T₂ of empty plasmid transformed line, and the starting plant. 20 plants of each line were used and the results were averaged.

At day 7 after pollination in the maturation period, the test plants were determined for the puncture strength, specifically by using the AWOS-SL04 stem strength tester by inserting a test head having a cross-sectional area of 1.0 mm² vertically into the middle portion of the aboveground third internode along a short axial direction of the stem and reading and recording the test data.

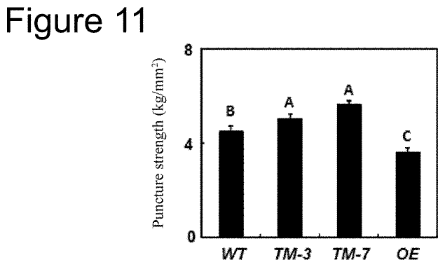

The results are shown in FIG. 11. Compared with the wild-type plants, the puncture strength of the stem is reduced by 22.15% for the plant of line OE, and increased by 18.97% for the plant of line TM-3 and by 21.45% for the plant of line TM-7. Compared with the wild-type plants, there is no significant change in the puncture strength of the stems of the plant of empty vector transformed line.

VI. Pot Experiment

The test seeds were seeds of generation T₃ of line OE, seeds of generation T₃ of line TM-3, seeds of generation T₃ of line TM-7, seeds of generation T₃ of empty plasmid transformed line, and seeds of the starting plant.

Plastic flower pots (32.5 cm in diameter*26 cm in height) were taken, and 14.0 kg of soil was filled in each pot. Then, the base fertilizer was applied, and the test seeds after germination were planted in pots and cultured in open air.

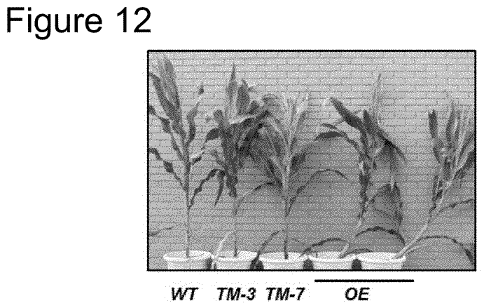

FIG. 12 shows a photograph of plants in the tasseling stage. When blown with wind, the plants of line OE line show bent stems (indicating that the mechanical strength of the stems became smaller), while the plants of lines TM-3 and TM-7, the wild-type plants, and the plants of empty vector transformed line remain upright (indicating that the mechanical strength of the stem is higher.

### Example 5: Determination of Target Gene

I. 5′RACE Test

8 genes were preliminarily predicted to be the candidate target genes of the miRNA having a sequence as shown in SEQ ID NO: 15 or 9, including GRMZM2G367668, GRMZM2G169033, GRMZM2G148937, GRMZM2G178741, GRMZM2G039381, GRMZM2G062069, GRMZM2G043300, and GRMZM2G004106, and each candidate target gene was sequentially verified.

The total RNA of maize B73 was extracted, and the 5′RACE test was performed on each candidate target gene by using GeneRacer™ Kit. The process included ligation of a 5′ RACE linker to the target gene mRNA product, reverse transcription into cDNA, nested PCR and cloning and sequencing of specific fragment. See instructions of use for details.

The results show that the cleavage site of the miRNA is present on the GRMZM2G367668 transcript and the GRMZM2G169033 transcript. The corresponding DNA of the GRMZM2G367668 transcript is shown in SEQ ID NO: 16 and the cleavage site of the miRNA is located between nucleotides 222-242 of the GRMZM2G367668 transcript. The corresponding DNA of the GRMZM2G169033 transcript is shown in SEQ ID NO: 17, and the cleavage site of the miRNA is located between nucleotides 302-322 of the GRMZM2G169033 transcript.

II. Northern Blot Analysis

The test plants were plants of generation T₂ of line OE, plants of generation T₂ of line TM-3, plants of generation T₂ of line TM-7, plants of generation T₂ of empty plasmid transformed line produced in Example 4, and the starting plant.

The roots of the test plants at the seedling stage were taken and total RNA was extracted and subjected to the Northern Blot analysis (where the probe for the GRMZM2G169033 transcript is shown in SEQ ID NO: 18, and the probe for the GRMZM2G367668 transcript is shown in SEQ ID NO: 19).

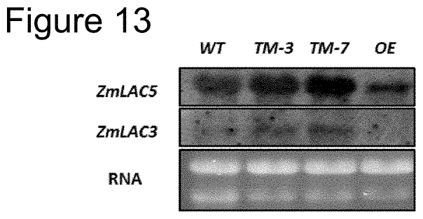

The results are shown in FIG. 13. In the plants of line OE, the level of the GRMZM2G169033 and GRMZM2G367668 transcripts are significantly decreased. The levels of the GRMZM2G169033 and GRMZM2G367668 transcripts are significantly increased in the plants of lines TM-3 and TM-7. The results indicate that the GRMZM2G169033 transcript and the GRMZM2G367668 transcript are target genes of the miRNA.

### Example 6: MicroRNA528 Affects Lodging Resistance of Maize by Regulating Lignin Biosynthesis Under Nitrogen-Luxury Conditions

I. Lignin Composition and Content of Maize Seedlings are Affected by N Supply.

As noted earlier, lodging under N-luxury conditions reduces maize yields in China. Because lodging could be related to lignin, and because quantitative data on maize lignin composition and content under different N conditions are lacking, we first determined the effects of N on maize lignin composition using thioacidolysis. Compared with N sufficiency, N deficiency substantially induced the generation of H, G, and S monomers in the shoots of maize seedlings (Table 2). N luxury, in contrast, significantly suppressed the generation of these monomers (Table 2). In addition, the S/G ratio in shoots was high when N supply was limited and low when N supply was excessive (Table 2).

TABLE 2
Lignin composition and content of maize seedlings under different N conditions.

| N | H | G | S | S/G molar | AcBr lignin |
|---|---|---|---|---|---|
| supply | (μmol/g) | (μmol/g) | (μmol/g) | ratio | (mg/g DW) |
| NL | 1.76 ± 0.07c | 15.50 ± 4.72c | 3.82 ± 1.72c | 0.23 ± 0.03c | 83.94 ± 1.98c |
| NS | 2.17 ± 0.13b | 23.48 ± 2.04b | 6.92 ± 0.87b | 0.29 ± 0.02b | 89.58 ± 1.81b |
| ND | 3.41 ± 0.45a | 38.68 ± 7.45a | 15.30 ± 2.92a | 0.40 ± 0.01a | 101.70 ± 3.30a |

NL, NS, and ND indicate N luxury, N sufficiency, and N deficiency, respectively. H, G, and S are p-coumaryl alcohol, coniferyl alcohol, and sinapyl alcohol, respectively. DW, dry weight. Values are means±SE of four biological replicates. Means with the same letter are not significantly different at p<0.01 according to the LSD test.

We also used acetyl bromide (AcBr) analysis (Iiyama and Wallis, 1990) to quantify the effects of N supply on the total lignin content of maize seedlings. In agreement with thioacidolysis, AcBr analysis showed that the total lignin content in the shoots of maize seedlings was reduced by N luxury but was enhanced by N deficiency (Table 2). The thioacidolysis and AcBr results were confirmed by phloroglucinol staining of 30-day-old hydroponically grown maize stems and leaves. Together, these results indicate that the lignin composition and content of maize are affected by N supply.

II. ZmmiR528 is Regulated by N Supply

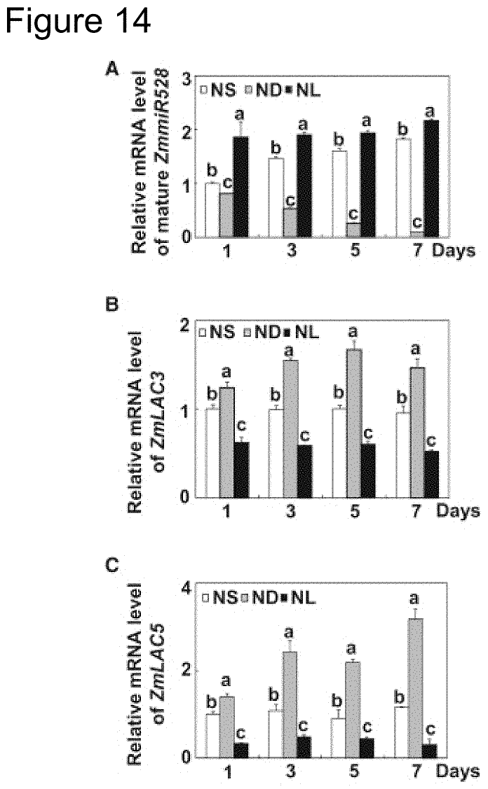

There are two members of the ZmmiR528 family in maize, ZmMIR528a and ZmMIR528b, and their mature sequences are identical. Several motifs recognized by N-responsive transcription factors, including TGA1/TGA4 and NAC4 (Vidal et al., 2013, Alvarez et al., 2014), could be found in the 1.5-kb promoter regions of ZmMIR528a and ZmMIR528b. Our previous sequencing data also showed that both ZmMIR528a and ZmMIR528b are significantly downregulated by N deficiency (Zhao et al., 2012). To determine how the expression of ZmmiR528 is regulated by N supply, we conducted a time-course experiment with maize grown hydroponically under N luxury and N deficiency. Stem-loop qRT-PCR showed that the expression of ZmmiR528 in shoots was induced by N luxury but was reduced by N deficiency (FIG. 14A), which is opposite to the changes in total lignin content in response to N luxury and deficiency (Table 2). In other words, N luxury increased ZmmiR528 expression and reduced total lignin content, while N deficiency decreased ZmmiR528 expression and increased total lignin content.

III. ZmLAC3 and ZmLAC5 are the Targets of ZmmiR528

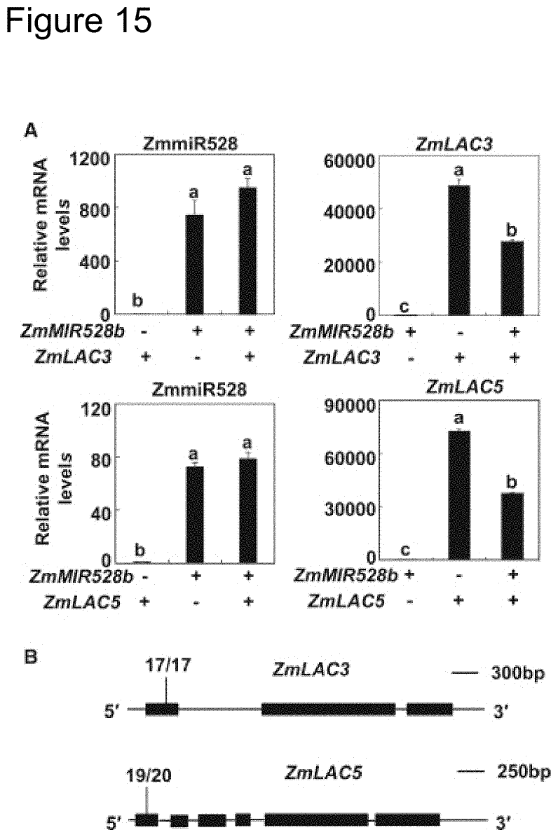

The potential targets of ZmmiR528 include ZmLAC3, ZmLAC5, several cupredoxins (GRMZM2G043300, GRMZM2G004106, and GRMZM2G039381), and a homeobox-transcription factor 22 ZmHB22 (GRMZM2G178741) (Zhang et al., 2009). Only ZmLAC3 and ZmLAC5 showed expression patterns opposite to those of ZmmiR528 in response to N-luxury and N-deficiency conditions (FIGS. 14B and C) indicating that the cupredoxins and ZmHB22 are not the targets of ZmmiR528 or that they are regulated by ZmmiR528 at the translational level. Thus, we selected ZmLAC3 and ZmLAC5 for further analysis. To determine whether ZmmiR528 can regulate ZmLAC3 and ZmLAC5 expression, we performed transient co-expression assays in Nicotiana benthamiana. After 2 days of co-expression in N. benthamiana, RNA was extracted, and ZmLAC3 and ZmLAC5 transcripts were analyzed by qRT-PCR. The abundance of ZmLAC3 and ZmLAC5 transcripts was significantly decreased when co-expressed with ZmMIR528b (FIG. 15A). In addition, the predicted target sites in ZmLAC3 and ZmLAC5 were determined by performing a 5′ RACE (rapid amplification of cDNA ends) assay using RNA from maize seedlings (FIG. 15B). Taken together, these results show that ZmLAC3 and ZmLAC5 are the authentic targets of ZmmiR528 in maize.

IV. ZmmiR528 is Mainly Expressed in Vascular Tissues

Because information on the spatial distribution of ZmmiR528 can provide insight into its function, we investigated the expression pattern of ZmmiR528 in different maize tissues by qRT-PCR. ZmmiR528 was expressed highly in the tenth leaf (from bottom), moderately in internodes, and at low levels in roots of adult maize plants. By contrast, the expression pattern of ZmLAC3 and ZmLAC5 was opposite to that of ZmmiR528, except that ZmLAC5 was also highly expressed in internodes.

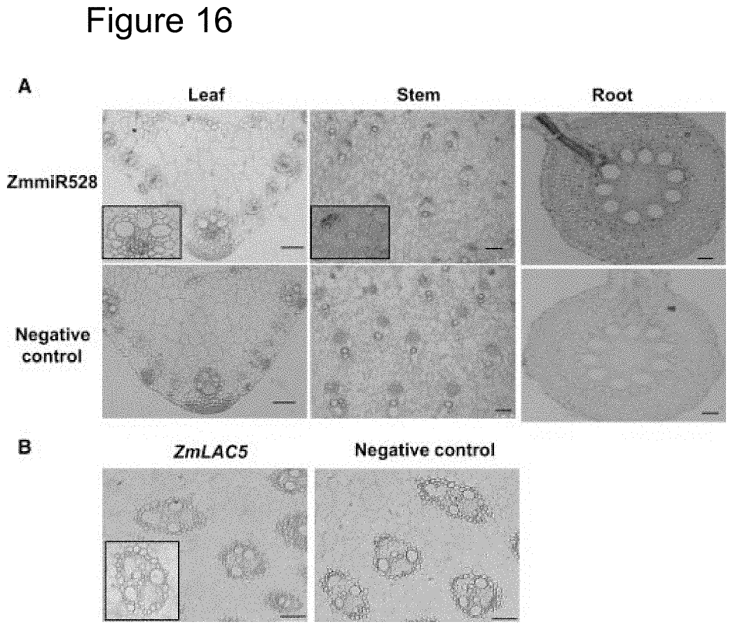

The expression pattern of ZmmiR528 was further examined by in situ hybridization analysis. ZmmiR528 was specifically detected in the phloem of maize leaves and stems and in the vascular region of lateral roots (FIG. 16A). Because the expression level of ZmLAC5 was also high in maize internodes, the expression pattern of ZmLAC5 in maize stems was also examined by in situ hybridization. Unlike ZmmiR528, ZmLAC5 transcript accumulated in the fibers of maize stems (FIG. 16B), further indicating the negative regulation of ZmLACs by ZmmiR528.

V. ZmmiR528 Affects Maize Lodging Resistance Under N-Luxury Conditions

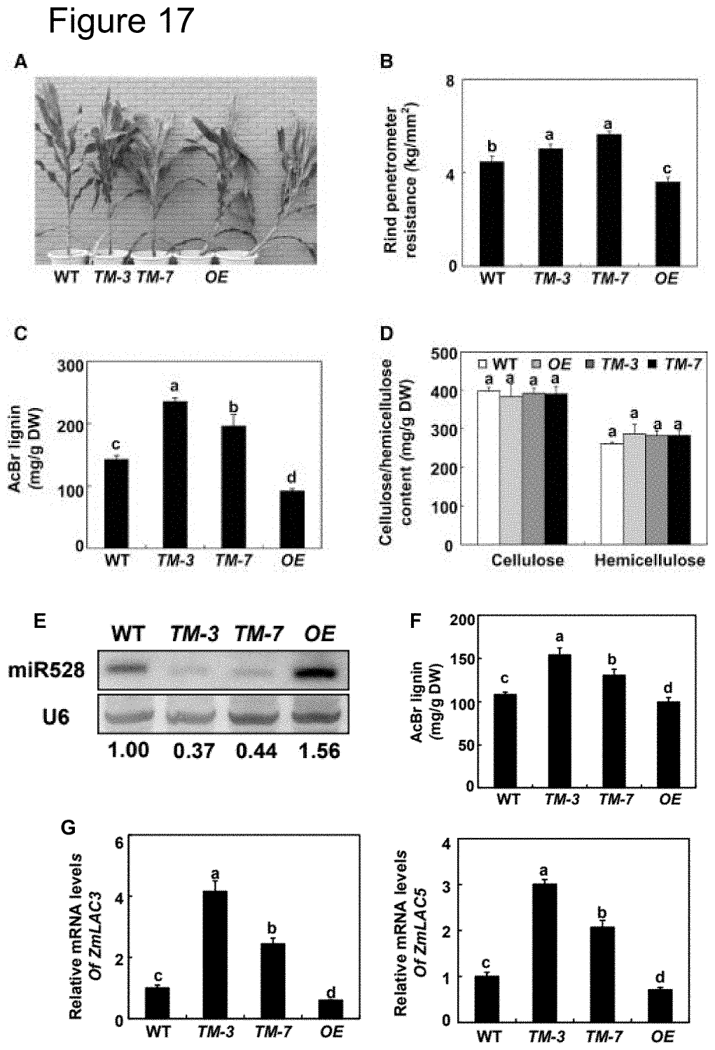

To characterize the function of ZmmiR528, we generated transgenic maize lines overexpressing ZmmiR528 under the control of the constitutive ubiquitin promoter. Only one line (OE) was obtained (FIG. 17E) due to the high level of ZmmiR528 expression in wild-type (WT) maize (Zhao et al., 2012). Because the ZmmiR528 family has two members, it is difficult to generate ZmmiR528 loss-of-function mutants using the CRISPR/Cas9 system. Thus, short tandem target mimics targeting ZmMIR528a and ZmMIR528b were designed as described by Yan et al. (2012) and were introduced into maize. The effectiveness of ZmMIR528a and ZmMIR528b destruction was verified by small RNA northern blot analysis, and two independent knockdown lines (TM-3 and TM-7) were chosen for subsequent analysis (FIG. 17E). The mRNA abundance of ZmLAC3 and ZmLAC5 was decreased by ZmmiR528 overexpression but was increased by ZmmiR528 knockdown (FIG. 17G), further demonstrating that ZmLAC3 and ZmLAC5 are directly regulated by ZmmiR528.

The regulation of ZmmiR528 by N supply and its specific expression in vascular tissues prompted us to analyze its potential role in lignin biosynthesis. Interestingly, ZmmiR528 abundance was negatively correlated with the lignin content in hydroponically grown maize seedlings, but not with cellulose and hemicellulose contents (FIG. 17F). Lignin content was ˜1.5 times greater in TM transgenic maize than in the WT (FIG. 17F). In addition, cell number was lower and cell diameter was larger in the phloem of OE transgenic maize stems than in the phloem of WT or TM transgenic maize, suggesting that phloem cells are arranged more loosely in OE transgenic maize. Thus, we evaluated the lodging resistance of the transgenic maize plants under N-luxury conditions. OE transgenic maize was more prone to lodging than the WT or TM transgenic maize under N-luxury conditions (FIG. 17A). In agreement with this phenotype, the rind penetrometer resistance and AcBr lignin content were much lower in OE transgenic maize than in the WT or TM transgenic maize (FIGS. 17B and C). The cellulose and hemicellulose contents in TM and OE transgenic maize were similar to those in WT (FIG. 17D). These results indicate that ZmmiR528 affects lodging resistance in maize under N-luxury conditions by affecting lignin content.

VI. Overexpression of ZmLAC3 Increases Lignin Content in Maize

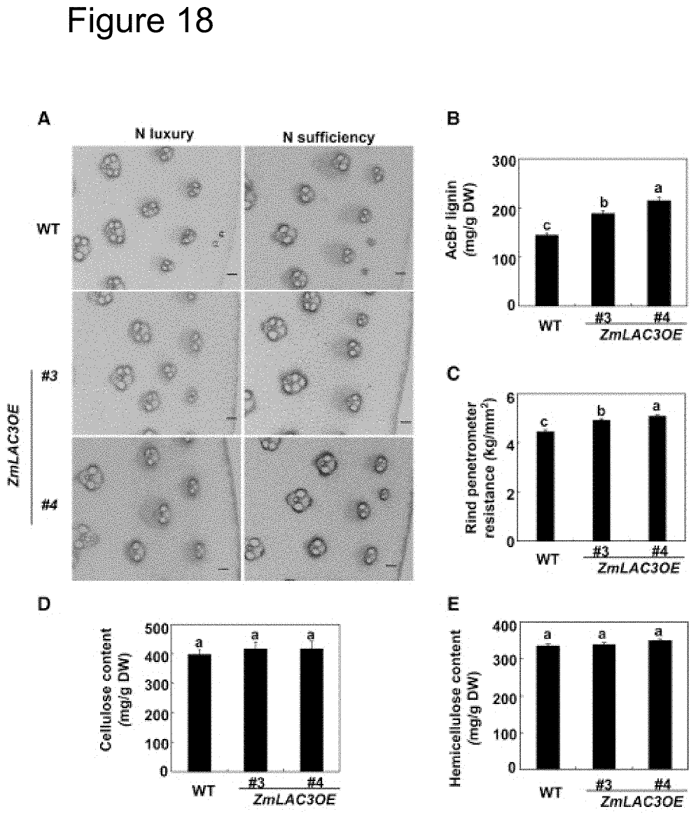

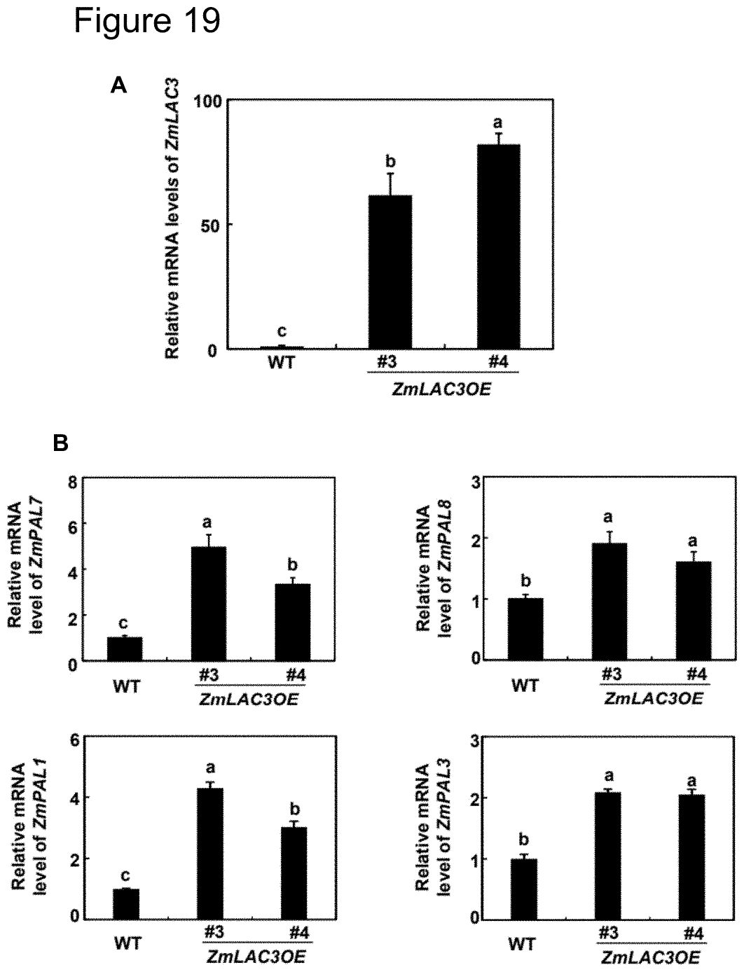

Laccases are copper-containing glycoproteins that are found in many organisms. It has been demonstrated that laccase is necessary and functions non-redundantly with peroxidase in lignin polymerization during vascular development in Arabidopsis (Zhao et al., 2013). To further characterize the function of ZmmiR528 in maize lignin biosynthesis, we generated transgenic maize plants overexpressing ZmLAC3, one of the ZmmiR528 targets, under the control of the constitutive ubiquitin promoter. Two transgenic lines (#3 and #4) were chosen for further analysis based on their high level of ZmLAC3 expression (FIG. 19A). Phloroglucinol staining of stems and leaves of WT and ZmLAC3OE transgenic maize seedlings showed that overexpression of ZmLAC3 increased the lignin content (FIG. 18A). For example, ectopic deposition of lignin under N-luxury and N-sufficient conditions was evident in the stem epidermis of ZmLAC3OE transgenic maize but not in the stem epidermis of WT maize (FIG. 18A). Based on AcBr analysis, the lignin content in mature stems under N-luxury conditions was 11%-20% higher in ZmLAC3OE transgenic maize than in the WT (FIG. 18B). The rind penetrometer resistance was also higher in ZmLAC3OE transgenic maize than in the WT maize (FIG. 18C). Consistent with the observation in ZmmiR528 TM transgenic maize, overexpression of ZmLAC3 did not affect the cellulose and hemicellulose contents compared with WT maize under N-luxury conditions (FIGS. 18D and E).

VII. ZmmiR528 and N Supply Affect the Expression of ZmPAL

To gain insight into the molecular events in the ZmmiR528-mediated signaling pathway, we compared the whole-transcriptome profiles of WT and ZmmiR528 TM transgenic maize using RNA sequencing (RNA-seq). Total RNA was extracted from the leaves of 15-day-old hydroponically grown inbred lines and transgenic maize supplied with sufficient N. There were three biological replicates for each genotype, and the libraries were sequenced using Illumina high-throughput sequencing technology. These six RNA libraries yielded more than 0.28 billion raw reads. Sequences that could not be mapped to the maize genome were discarded, and only those that were perfectly mapped were analyzed further. The abundance of each gene was expressed as fragments per kilobase per million mapped reads (Trapnell et al., 2010). Approximately 2328 genes showed statistically significant changes in expression in ZmmiR528 TM transgenic maize compared with WT under non-stress conditions. Of the differentially expressed genes (DEGs), 1048 were downregulated and 1281 were upregulated in ZmmiR528 TM transgenic maize. By performing BLAST searches against the UniProt database (http://www.uniprot.org/blast/), we functionally annotated 1932 of the DEGs. Among the DEGs, UDP-glycosyltransferase 72B1 (UGT72B1), was reported to catalyze the glucose conjugation of monolignols and is essential for normal cell wall lignification in Arabidopsis (Lin et al., 2016). AtABCG29 is a p-coumaryl alcohol transporter involved in lignin biosynthesis (Alejandro et al., 2012). The transcripts of the corresponding orthologs of these genes in maize, GRMZM2G156026 and GRMZM2G366146, were significantly upregulated in ZmmiR528 TM transgenic maize. Gene Ontology (GO; http://bioinfo.cau.edu.cn/agriGO/) analysis indicated that the annotated DEGs are mostly involved in GO:0006950 (response to stress; p=1.70E-6), GO:0019748 (secondary metabolic process; p=6.80E-6), GO:0044271 (cellular nitrogen compound biosynthetic process; p=0.0008), GO:0005618 (cell wall; p=2.60E-14), and GO:0030054 (cell junction; p=5.60E-10).

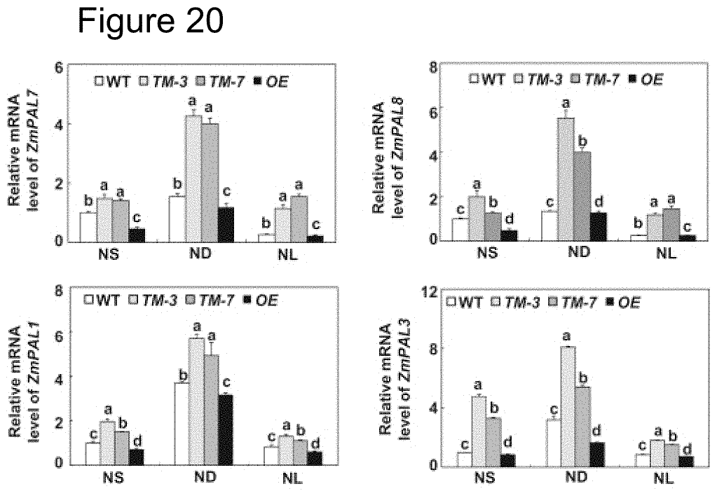

GRMZM2G170692 and GRMZM2G334660 attracted our attention because they encode phenylalanine ammonia lyase 7 (PAL7) and PAL8, respectively. PAL not only catalyzes the first step in a series of enzymatic reactions generating monolignols from phenylalanine but is also a key link in the phenylpropanoid-nitrogen cycle (Cantón et al., 2005, Vanholme et al., 2008). Under N-sufficient conditions, ZmmiR528 negatively affected ZmPAL7 and ZmPAL8 expression, i.e., ZmPAL7 and ZmPAL8 mRNA levels were highest in ZmmiR528 TM transgenic maize, followed in order by WT and ZmmiR528 OE transgenic maize (FIG. 20). These results were further verified in ZmLAC3OE transgenic maize (FIG. 19B). N deficiency significantly induced ZmPAL7 and ZmPAL8 expression in WT, ZmmiR528 OE, and TM transgenic maize, with the highest induction observed in ZmmiR528 TM transgenic maize (FIG. 20). Under N-luxury conditions, ZmPAL7 and ZmPAL8 expression levels in ZmmiR528 TM transgenic maize were similar to those under N-sufficient conditions. In contrast, expression levels were significantly reduced in WT and ZmmiR528 OE transgenic maize under N-luxury conditions (FIG. 20). The expression levels of another seven ZmPALs were also determined in these transgenic maize lines by real-time RT-PCR. Consistent with ZmPAL7 and ZmPAL8, the mRNA levels of ZmPAL1 and ZmPAL3 were regulated by N supply, ZmmiR528, and ZmLAC3 abundance (FIGS. 20 and 19B).

VIII. Discussion

Although maize is an important model for studying plant development and evolution, research on maize has lagged behind research on rice and Arabidopsis, perhaps because of the low transformation efficiency and limited genetic background for transgenic maize. To date, the only functionally characterized miRNAs in maize are ZmmiR156 (Chuck et al., 2007a), ZmmiR164 (Li et al., 2012), ZmmiR166 (Juarez et al., 2004), and ZmmiR172 (Lauter et al., 2005, Chuck et al., 2007b). In the present study, we found that ZmmiR528, by negatively regulating the abundance of ZmLAC3 and ZmLAC5 mRNA, affects lignin biosynthesis and lodging resistance of maize under N-luxury conditions, providing significant insights into the relationship between N and lignin biosynthesis.

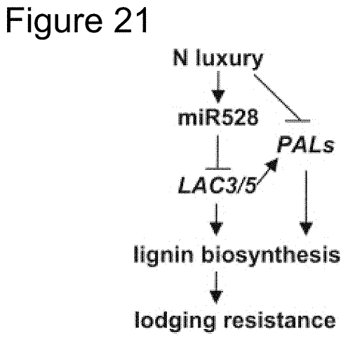

Lodging under N-luxury conditions is a major problem limiting maize yield in China. In poplar, lignin in the secondary cell walls of the xylem was reduced under N-luxury conditions but was consistently deposited under N-limiting conditions (Camargo et al., 2014). In forage grasses, the lignin content is increased by N fertilization (Collins et al., 1990). Here, we quantified the effects of N treatment on lignin composition and content in maize. N luxury significantly suppressed the generation of H, G, and S monomers and total lignin content in maize seedlings. The TGA1/4 and NAC4 transcription factors are key regulators of the nitrate-responsive network (Vidal et al., 2013, Alvarez et al., 2014). Binding sites corresponding to these transcription factors were found in the 1.5-kb promoter regions of ZmMIR528a/b, indicating that ZmMIR528a/b expression could be regulated by N treatment, and stem-loop qRT-PCR verified this hypothesis. ZmMIR528a/b could directly cleave ZmLAC3 and ZmLAC5, and the lignin content was especially enhanced in ZmmiR528 knockdown mutants and ZmLAC3-overexpressing transgenic maize. Ectopic deposition of lignin was evident in the stem epidermis of these lines. Although monolignol biosynthesis genes were little affected in the Arabidopsis Iac4Iac11Iac17 triple mutant (Zhao et al., 2013), knockdown of ZmmiR528 and overexpression of ZmLAC3 significantly increased PAL expression levels in the current study. Under N-luxury conditions, ZmPAL expression levels were much higher in ZmmiR528 TM transgenic maize than in WT or ZmmiR528 OE transgenic maize. Taken together, the increased levels of miR528 and decreased abundance of ZmLACs and ZmPALs could explain the reduced lodging resistance of maize under N-luxury conditions (FIG. 21).

As noted earlier, miRNA528 is a monocot-specific miRNA. Although the mature sequence of miRNA528 (5′-UGG AAG GGG CAU GCA GAG GAG-3′) is the same in rice and maize, the function of miRNA528 in these species differs. In rice, OsmiR528 contributes to the immune response by downregulating an AO (Wu et al., 2017). We used co-expression in N. benthamiana and 5′ RACE to determine that in maize, in contrast to rice, ZmLAC3 and ZmLAC5 are the authentic targets of ZmmiR528. Overexpression of ZmLAC3 increased the lignin content of maize, which is consistent with the phenotypes of ZmMIR528a/b TM transgenic maize. In addition, ZmmiR528 is specifically expressed in the phloem of maize leaves and stems. Taken together, our results indicate that by regulating ZmLAC3 and ZmLAC5, ZmmiR528 affects maize lignin biosynthesis.

The transcriptional regulation of laccases is important for secondary wall formation in Arabidopsis (Mitsuda et al., 2007, Zhou et al., 2009, Wang et al., 2010). Although Arabidopsis lacks miRNA528, it has other miRNAs that provide important functions related to lignin biosynthesis. For example, the overexpression of miRNA408 in Arabidopsis downregulates LAC13 and ARPN mRNA levels and increases vegetative growth (Zhang and Li, 2013); miRNA397b regulates both lignin content and seed number in Arabidopsis by modulating a laccase involved in lignin biosynthesis (Wang et al., 2014); and miRNA857 regulates the secondary growth of vascular tissues in Arabidopsis through regulation of LAC7 (Zhao et al., 2015). We therefore infer that the post-transcriptional regulation of lignin biosynthesis is conserved between monocotyledons and dicotyledons.

In addition to being a source of food for humans, maize is also an important raw material for biofuel production and an important forage crop for livestock. The use of maize for biofuel production requires the removal of lignin, and lignin affects the digestibility of forage crops (Zhou et al., 2009). Because ZmmiRNA528 OE transgenic maize has reduced lignin content, it could be useful for biofuel production or forage. Most importantly, the results of the current study indicate that ZmmiR528 and ZmLAC modules might be modified to reduce the lodging of maize under N-luxury conditions.

IX. Methods

### Plant Materials and Growth Conditions

Seeds of uniform size were surface sterilized in 10% H₂O₂ for 20 min. After they were soaked in a saturated CaSO₄ solution with continuous aeration for 6 h, the seeds were germinated in coarse quartz sand until two leaves were visible. The seedlings were grown hydroponically as described by Zhao et al. (2012) to assess responses to N-sufficient (4 mM NO₃—, the concentration in full-strength Hoagland's nutrient solution), N-luxury (8 mM NO₃—), or N-deficient conditions (0.04 mM NO₃—). Maize was also grown in soil to determine lodging resistance under N-luxury conditions. For soil culture, plants were grown in pots containing 10 kg of soil for 70 days under natural conditions. Before planting, 6.2 g of urea, 12.1 g of calcium superphosphate, and 5.5 g of potassium sulfate were mixed into the soil of each pot. The quantity of urea fertilizer was about two times higher in the pot experiment than in practical inputs. At the maize V6 stage, an additional 2 g of urea was applied to each pot.

Constructs and Generation of Transgenic Maize

The cDNA of ZmLAC3 without the 3′ UTR was amplified by PCR. A ˜80-bp fragment surrounding the ZmMIR528b sequence including the fold-back structure was amplified from aenomic DNA. To generate the ZmmiR528 knockdown mutant ZmmiR528 TM, we designed short tandem target mimics targeting ZmMIR528a and ZmMIR528b as described by Yan et al. (2012). The amplified fragments were cloned into the pCUB vector under the control of the rice ubiquitin promoter using the BamHI restriction site via an In-Fusion reaction.

The constructed plasmid containing the Streptomyces hygroscopicus phosphinothricinacetyltransferase (bar) gene under the control of a CaMV 35S promoter was electroporated into A. tumefaciens EHA105. Immature embryos of maize inbred line ZZC01 were transformed by co-cultivation with EHA105 at the Life Science and Technology Center of China National Seed Group. Transformants were selected with gradually increasing concentrations of bialaphos. More than 20 independent transgenic events were generated, and events with a single copy or low copy numbers were advanced for selfing or crossing with the maize inbred line ZZC01 to produce T₁ seeds. Following real-time qRT-PCR analysis, T₁ plants with confirmed transgene expression were self-pollinated to generate T₂seeds. T₂ homozygous lines were used for all experiments.

Analysis of Lignin, Cellulose, and Hemicellulose Contents

Different parts of 15-day-old hydroponically grown maize plants and the third internode from the bottom of soil-grown maize at the V9 growth stage were sampled. The samples were dried at 45° C. for 7 days and then milled to a fine powder. Lignin composition was determined by thioacidolysis (Lapierre et al., 1995). The AcBr method was used to determine the total lignin content as described by Fukushima and Hatfield (2004). Cellulose and hemicellulose contents were determined using the modified NREL procedures (Sluiter et al., 2008).

Histochemical Analysis of Vascular Structure

We sampled the leaves in the same position of 15-day-old hydroponically grown maize and the first 2 cm of the stem above the roots of 30-day-old hydroponically grown maize. The vascular structure of these samples was visualized by Wiesner staining as previously described (Berthet et al., 2011).

RNA Analysis

Total RNA was extracted from inbred lines and transgenic maize using TRIzol reagent (Invitrogen). For quantification of ZmmiR528, stem-loop qRT-PCR was performed as previously described (Chen et al., 2005). The real-time RT-PCR and cleavage site mapping were performed as described by Zhao et al. (2012).

Transient Expression in N. benthamiana

Full-length cDNA sequences of ZmLAC3, ZmLAC5, and ZmMIR528b including the fold-back structure were amplified with the primers. The amplified fragments were cloned into the pCPB vector using the BamHI restriction site via an In-Fusion reaction. The plasmids were electroporated into A. tumefaciens GV3101 and were transiently expressed in tobacco epidermal cells as described by Li et al. (2008). Leaves were harvested 2 days after the infiltration and were subjected to RNA analysis as described above.

In Situ Hybridization

In situ hybridization of ZmmiR528 was performed as described by Trevisan et al. (2012). For the ZmLAC5 probe, a 385-bp fragment with high specificity was amplified and cloned into the pGEM-T-easy vector (Promega). The corresponding sense and antisense probes were generated by in vitro transcription using T7 or SP6 RNA polymerases. The in situ hybridization of ZmLAC5 was performed as previously described (Zhang et al., 2007).

### Measurement of Breaking Force

The third internodes from the bottom of soil-grown maize at the V9 growth stage were used for measurements. The force required to break the stems was recorded with a microtester (AWOS-SL04). Ten plants of each genotype were measured, and all measurements were taken under the same conditions.

### Cell Number and Size

For the determination of phloem cell number and diameter for each genotype, the same part of the second leaf from the bottom was fixed overnight at 4° C. in 2.5% glutaraldehyde in 0.1 M phosphate buffer (pH 7.2) and post-fixed for 2 h in 1% OsO₄. The electron tomograms were collected with an HT7700 transmission electron microscope (Hitachi) operating at 80 kV. Image Pro Plus 6.0 was used to measure phloem cell diameter.

### Example 7: CRISPR/Cas9-Mediated miRNA528 a and b Editing

Two separate pairs of gRNAs of MIRNA528 were produced:

528a:
(SEQ ID NO: 33)
TCAGGGTCAGTTTGCTCTGCTGG,
(SEQ ID NO: 36)
TGCGCAGTTGCCCTGTGATGAGG
528b:
(SEQ ID NO: 40)
TCTCTAGTTCTGGGAGTTCCTGG
and
(SEQ ID NO: 43)
AAAACTCAGAAGCGCAACCGAGG

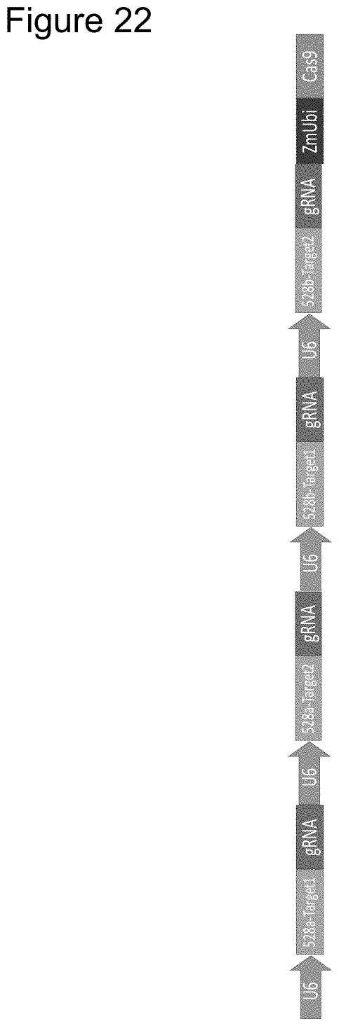

Thereafter, the fragments were cloned into the pCPB vector using the HindIII restriction site by In-Fusion reaction. The schematic diagrams of sgRNAs and hSpCas9 are provided in FIG. 22.

### Example 9: Field-Study

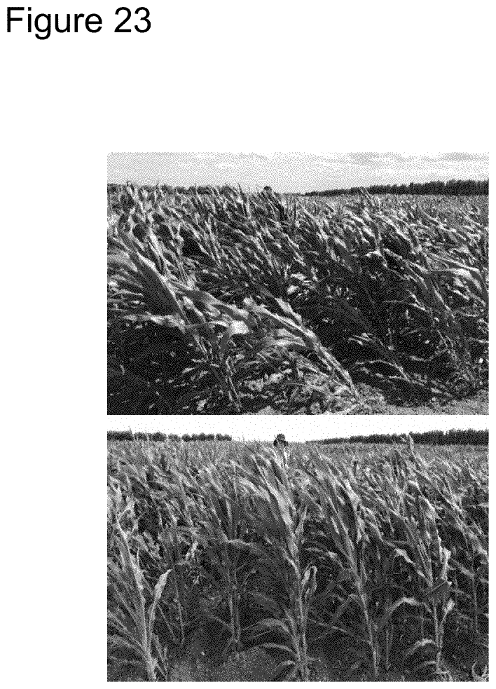

We made miR528 knockdown transgenetic (female) plants and crossed these with males prone to lodging. The F1 generation was then backcrossed with lodging prone inbreds. As shown in the photograph in FIG. 23, the resulting plants had increased lodging in the field (when exposed to lodging conditions), compared to wild-type that had been crossed with lodging-prone plants (WTxhybrids) without any negative phenotype, such as delayed flowering time. The lodging resistant plants could be used as male or female parents to generate new hybrids.

### REFERENCES

S. Alejandro, Y. Lee, T. Tohge, D. Sudre, S. Osorio, J. Park, L. Bovet, Y. Lee, N. Geldner, A. R. Fernie, et al. AtABCG29 is a monolignol transporter involved in lignin biosynthesis. Curr. Biol., 22 (2012), pp. 1207-1212
J. M. Alvarez, E. Riveras, E. A. Vidal, D. E. Gras, O. Contreras-López, K. P. Tamayo, F. Aceituno, I. Gómez, S. Ruffel, L. Lejay, et al. Systems approach identifies TGA1 and TGA4 transcription factors as important regulatory components of the nitrate response of Arabidopsis thaliana roots. Plant J., 80 (2014), pp. 1-13
J. Barros, H. Serk, I. Granlund, E. Pesquet The cell biology of lignification in higher plants. Ann. Bot., 115 (2015), pp. 1053-1074
S. Berthet, N. Demont-Caulet, B. Pollet, P. Bidzinski, L. Cézard, P. Le Bris, N. Borrega, J. Hervé, E. Blondet, S. Balzergue, et al. Disruption of LACCASE4 and 17 results in tissue-specific alterations to lignification of Arabidopsis thaliana stems. Plant Cell, 23 (2011), pp. 1124-1137
N. H. Bhuiyan, G. Selvaraj, Y. Wei, J. King Role of lignification in plant defense. Plant Signal. Behav., 4 (2009), pp. 158-159
W. Boerjan, J. Ralph, M. Baucher. Lignin biosynthesis. Ann. Rev. Plant Biol., 54 (2003), pp. 519-546
A. C. Bryan, S. Jawdy, L. Gunter, E. Gjersing, R. Sykes, M. A. Hinchee, K. A. Win keler, C. M. Collins, N. Engle, T. J. Tschaplinski, et al. Knockdown of a laccase in Populus deltoides confers altered cell wall chemistry and increased sugar release. Plant Biotechnol. J., 14 (2016), pp. 2010-2020
E. L. Camargo, L. C. Nascimento, M. Soler, M. M. Salazar, J. Lepikson-Neto, W. L. Marques, A. Alves, P. J. Teixeira, P. Mieczkowski, M. F. Carazzolle, e t al. Contrasting nitrogen fertilization treatments impact xylem gene expression and secondary cell wall lignification in Eucalyptus. BMC Plant Biol., 14 (2014), p. 256
F. R. Cantón, M. F. Suárez, F. M. Cánovas. Molecular aspects of nitrogen mobilization and recycling in trees. Photosynth. Res., 83 (2005), pp. 265-278 C. Chen, D. A. Ridzon, A. J. Broomer, Z. Zhou, D. H. Lee, J. T. Nguyen, M. Barbis in, N. L. Xu, V. R. Mahuvakar, M. R. Andersen, et al. Real-time quantification of microRNAs by stem-loop RT-PCR. Nucleic Acids Res., 33 (2005), p. e179
M. Collins, M. A. Brinkman, A. A. Salman Forage yield and quality of oat cultivars with increasing rates of nitrogen fertilization. Agron. J., 82 (1990), pp. 724-728
G. Chuck, A. M. Cigan, K. Saeteurn, S. Hake The heterochronic maize mutant corngrass1 results from overexpression of a tandem microRNA. 50 Nat. Genet., 39 (2007), pp. 544-549
G. Chuck, R. Meeley, E. Irish, H. Sakai, S. Hake The maize tasselseed4 microRNA controls sex determination and meristem cell fate by targeting Tasselseed6/indeterminate spikelet. Nat. Genet., 39 (2007), pp. 1517-1521
R. S. Fukushima, R. D. Hatfield. Comparison of the acetyl bromide spectrophotometric method with other analytical lignin methods for determining lignin concentration in forage samples. J. Agric. Food Chem., 52 (2004), pp. 3713-3720
J. H. Guo, X. J. Liu, Y. Zhang, J. L. Shen, W. X. Han, W. F. Zhang, P. Christie, K. W. Goulding, P. M. Vitousek, F. S. Zhang. Significant acidification in major Chinese croplands. Science, 327 (2010), pp. 1008-1010
K. liyama, A. F. A. Wallis. Determination of lignin in herbaceous plants by an improved acetyl bromide procedure. J. Sci. Food Agric., 51 (1990), pp. 145-161
X. T. Ju, G. X. Xing, X. P. Chen, S. L. Zhang, L. J. Zhang, X. J. Liu, Z. L. Cui, B. Yin, P. Christie, Z. L. Zhu, et al. Reducing environmental risk by improving N management in intensive Chinese agricultural systems. Proc. Natl. Acad. Sci. USA, 106 (2009), pp. 3041-3046
M. T. Juarez, J. S. Kui, J. Thomas, B. A. Heller, M. C. P. Timmermans. microRNA-mediated repression of rolled leaf1 specifies maize leaf polarity. Nature, 428 (2004), pp. 84-88
S. Khan, S. Anwar, J. Kuai, A. Noman, M. Shahid, M. Din, A. Ali, G. Zhou. Alteration in yield and oil quality traits of winter rapeseed by lodging at different planting density and nitrogen rates. Sci. Rep., 8 (2018), p. 634.
C. Lapierre, B. Pollet, C. Rolando. New insights into the molecular architecture of hardwood lignins by chemical degradative methods. Res. Chem. Intermed., 21 (1995), pp. 397-412
N. Lauter, A. Kampani, S. Carlson, M. Goebel, S. P. MoosemicroRNA172 down-regulates glossy15 to promote vegetative phase change in maize. Proc. Natl. Acad. Sci. USA, 102 (2005), pp. 9412-9417
J. Li, G. Guo, W. Guo, G. Guo, D. Tong, Z. Ni, Q. Sun, Y. Yao. miRNA164-directed cleavage of ZmNAC1 confers lateral root development in maize (Zea mays L.). BMC Plant Biol., 12 (2012), p. 220
W. X. Li, Y. Oono, J. Zhu, X. J. He, J. M. Wu, K. Iida, X. Y. Lu, X. Cui, H. Jin, J. K. Zhu The Arabidopsis NFYA5 transcription factor is regulated transcriptionally and posttranscriptionally to promote drought resistance. Plant Cell, 20 (2008), pp. 2238-2251
J. S. Lin, X. X. Huang, Q. Li, Y. Cao, Y. Bao, X. F. Meng, Y. J. Li, C. Fu, B. K. Hou UDP-glycosyltransferase 72B1 catalyzes the glucose conjugation of monolignols and is essential for the normal cell wall lignification in Arabidopsis thaliana. Plant J., 88 (2016), pp. 26-42
X. J. Liu, Y. Zhang, W. Han, A. Tang, J. Shen, Z. Cui, P. Vitousek, J. W. Erisman, K. Goulding, P. Christie, et al. Enhanced nitrogen deposition over China. Nature, 494 (2013), pp. 459-462
S. Lu, Q. Li, H. Wei, M. J. Chang, S. Tunlaya-Anukit, H. Kim, J. Liu, J. Song, Y. H. Sun, L. Yuan, et al. Ptr-miR397a is a negative regulator of laccase genes affecting lignin content in Populus trichocarpa. Proc. Natl. Acad. Sci. USA, 110 (2013), pp. 10848-10853
N. Mitsuda, A. Iwase, H. Yamamoto, M. Yoshida, M. Seki, K. Shinozaki, M. Oh me-Takagi NAC transcription factors, NST1 and NST3, are key regulators of the formation of secondary walls in woody tissues of Arabidopsis. Plant Cell, 19 (2007), pp. 270-280
A. N. Shah, M. Tanveer, A. U. Rehman, S. A. Anjum, J. Iqbal, R. Ahmad Lodging stress in cereal-effects and management: an overview. Environ. Sci. Pollut. Res. Int., 24 (2017), pp. 5222-5237
A. Sluiter, B. Hames, R. Ruiz, C. Scarlata, J. Sluiter, D. Templeton, D. Crocker. Determination of Structural Carbohydrates and Lignin in Biomass National Renewable Energy Laboratory (2008)
C. Trapnell, B. A. Williams, G. Pertea, A. Mortazavi, G. Kwan, M. J. van Baren, S. L. Salzberg, B. J. Wold, L. Pachter Transcript assembly and quantification by RNA-Seq reveals unannotated transcripts and isoform switching during cell differentiation. Nat. Biotechnol., 28 (2010), pp. 511-515
S. Trevisan, A. Nonis, M. Begheldo, A. Manoli, K. Palme, G. Caporale, B. Ruper ti, S. Quaggiotti. Expression and tissue-specific localization of nitrate-responsive miRNAs in roots of maize seedlings. Plant Cell Environ., 35 (2012), pp. 1137-1155
R. Vanholme, K. Morreel, J. Ralph, W. Boerjan. Lignin engineering Curr. Opin. Plant Biol., 11 (2008), pp. 278-285
H. Vaucheret. Post-transcriptional small RNA pathways in plants: mechanisms and regulations. Genes Dev., 20 (2006), pp. 759-771
E. A. Vidal, T. C. Moyano, E. Riveras, O. Contreras-López, R. A. Gutiérrez Systems approaches map regulatory networks downstream of the auxin receptor AFB3 in the nitrate response of Arabidopsis thaliana roots. Proc. Natl. Acad. Sci. USA, 110 (2013), pp. 12840-12845
W. Wang, J. Lu, T. Ren, X. K. Li, W. Su, M. X. Lu Evaluating regional mean optimal nitrogen rates in combination with indigenous nitrogen supply for rice production

### Field Crops Res., 137 (2012), pp. 37-48

C. Y. Wang, S. Zhang, Y. Yu, Y. C. Luo, Q. Liu, C. Ju, Y. C. Zhang, L. H. Qu, W. J. Lucas, X. Wang, et al. MiR397b regulates both lignin content and seed number in Arabidopsis via modulating a laccase involved in lignin biosynthesis. Plant Biotechnol. J., 12 (2014), pp. 1132-1142
H. Wang, U. Avci, J. Nakashima, M. G. Hahn, F. Chen, R. A. Dixon Mutation of WRKY transcription factors initiates pith secondary wall formation and increases stem biomass in dicotyledonous plants. Proc. Natl. Acad. Sci. USA, 107 (2010), pp. 22338-22343
J. Wu, R. Yang, Z. Yang, S. Yao, S. Zhao, Y. Wang, P. Li, X. Song, L. Jin, T. Zh ou, et al. ROS accumulation and antiviral defence control by microRNA528 in rice. Nat. Plants, 3 (2017), p. 16203
J. Yan, Y. Gu, X. Jia, W. Kang, S. Pan, X. Tang, X. Chen, G. Tang. Effective small RNA destruction by the expression of a short tandem target mimic in Arabidopsis. Plant Cell, 24 (2012), pp. 415-427
S. Yuan, Z. Li, D. Li, N. Yuan, Q. Hu, H. Luo. Constitutive expression of rice microRNA528 alters plant development and enhances tolerance to salinity stress and nitrogen starvation in creeping bentgrass. Plant Physiol., 169 (2015), pp. 576-593
H. Zhang, L. Li. SQUAMOSA promoter binding protein-like7 regulated microRNA408 is required for vegetative development in Arabidopsis Plant J., 74 (2013), pp. 98-109
J. Zhang, G. Li, Y. Song, Z. Liu, C. Yang, S. Tang, C. Zheng, S. Wang, Y. Ding Lodging resistance characteristics of high-yielding rice populations Field Crops Res., 161 (2014), pp. 64-74.
L. Zhang, J. M. Chia, S. Kumari, J. C. Stein, Z. Liu, A. Narechania, C. A. Maher, K. Guill, M. D. McMullen, D. Ware. A genome-wide characterization of microRNA genes in maize. PLoS Genet., 5 (2009), p. e1000716
X. Zhang, S. Madi, L. Borsuk, D. Nettleton, R. J. Elshire, B. Buckner, D. Janick-Buckner, J. Beck, M. Timmermans, P. S. Schnable, et al. Laser microdissection of narrow sheath mutant maize uncovers novel gene expression in the shoot apical meristem. PLoS Genet., 3 (2007), p. e101
W. Zhang, L. Wu, X. Wu, Y. Ding, G. Li, J. Li, F. Weng, Z. Liu, S. Tang, C. Ding, et al. Lodging resistance of Japonica rice (Oryza Sativa L.): morphological and anatomical traits due to top-dressing nitrogen application rates. Rice, 9 (2016), p. 31
M. Zhao, H. Tai, S. Sun, F. Zhang, Y. Xu, W. X. Li. Cloning and characterization of maize miRNAs involved in responses to nitrogen deficiency. PLoS One, 7 (2012), p. e29669
Q. Zhao, J. Nakashima, F. Chen, Y. Yin, C. Fu, J. Yun, H. Shao, X. Wang, Z. Y. Wang, R. A. Dixon Laccase is necessary and nonredundant with peroxidase 50 for lignin polymerization during vascular development in Arabidopsis. Plant Cell, 25 (2013), pp. 3976-3987
Y. Zhao, S. Lin, Z. Qiu, D. Cao, J. Wen, X. Deng, X. Wang, J. Lin, X. Li MicroR NA857 is involved in the regulation of secondary growth of vascular tissues in Arabidopsis

### Plant Physiol., 169 (2015), pp. 2539-2552

J. Zhou, C. Lee, R. Zhong, Z. H. YeMYB58 and MYB63 are transcriptional activators of the lignin biosynthetic pathway during secondary cell wall formation in Arabidopsis. Plant Cell, 21 (2009), pp. 248-266
U. Zuber, H. Winzeler, M. M. Messmer, M. Keller, B. Keller, J. E. Schmid, P. Sta mp Morphological traits associated with lodging resistance of spring wheat (Triticum aestivum L.) J. Agron. Crop Sci., 182 (1999), pp. 17-24
Zhao Y, Zhang C, Liu W, et al. An alternative strategy for targeted gene replacement in plants using a dual-sgRNA/Cas9 design[J]. Scientific Reports, 2016, 6:23890.
Miki D, Zhang W, Zeng W, et al. CRISPR/Cas9-mediated gene targeting in Arabidopsis using sequential transformation[J]. Nature Communications, 2018, 9(1):1967.
Cermak, T et al. Efficient design and assembly of custom TALEN and other TAL effector-based constructs for DNA targeting. Nucleic acid Res. 39 (2011).
Kunkel T A. 1985. Rapid and efficient dite-specifc mutagenesis without phenotypic selection. PNAS. 82(2): 488-92.
Kunkel T A, Roberts J D, Zakour R A. 1987. Rapid and efficient dite-specifc mutagenesis without phenotypic selection. Methods Enzmol. 154. 367-82.
Krysan P J, Young J C, Sussman M R. 1999. T-DNA as an insertional mutagen in Arabidopsis. Plant Cell. 11(12): 2283-90.
Henikoff S, Till B J, Comai L. 2004. TILLING. Traditional mutagenesis meets functional genomics. Plant Physiol. 135(2): 630-6.
Comai L, Young K, Till B J, Reynolds S H, Greene E A, Codoma C A, Enns L C, Johnson J E, Burtner C, Odden A R, Heinkoff. 2004. Efficient discovery of DNA polymorphisms in natural populations by Ecotilling. Plant J. 37(5):778-86.
Komor, A. C.; Kim, Y. B.; Packer, M. S.; Zuris, J. A.; Liu, D. R. 2-16. Programmable editing of a target base in genomic DNA without double-stranded DNA cleavage. Nature, 533, 420-424
Neville E Sanjana, Le Cong, Yang Zhou, Margaret M Cunniff, Guoping Feng& Feng Zhang. 2012. A transcription activator-like effector toolbox for genome engineering, Nature Protocols 7, 171-192.
Clough S J, Bent A F. 1998. Floral dip: a simplified method for Agrobacterium-mediated transformation of Arabidopsis thaliana. Plant J. 16(6): 735-43.
Ma X, Zhang Q, Zhu Q, Liu W, Chen Y, Qiu R, Wang B, Yang Z, Li H, Lin Y, Xie Y, Shen R, Chen S, Wang Z, Chen Y, Guo J, Chen L, Zhao X, Dong Z, Liu Y. 2015. A Robust CRISPR/Cas9 System for Convenient, High-Efficiency Multiplex Genome Editing in Monocot and Dicot Plants. 8(8): 1274-84.
Radhar M, McMahon M A, Prakash T P, Swayze E E, Bennett F, Cleveland D W. 2015. Synthetic CRISPR RNA-Cas9-guided genome editing in human cells. PNAS. 112(51) E7110-E7117.

SEQUENCE LISTING

SEQ ID NO: 1 Laccase 3 amino acid
MPLRQRPTMGGGGGGVAKMPAGQLWLLLLGVLLLAFGVPAQASRNTHYDFVITETKVTRL
CHEKTILAVNGQFPGPTIYARKDDVVIVNVYNQGYKNITLHWHGVDQPRNPWSDGPEYIT
QCPIQPGANFTYKIIFTEEEGTLWWHAHSEFDRATVHGAIVIHPKRGTVYPYPKPHKEMP
IILGEWWNADVEQILLESQRTGGDVNISDANTINGQPGDFAPCSKEDTFKMSVEHGKTYL
LRVINAGLTNEMFFAVAGHRLTVVGTDGRYLRPFTVDYILISPGQTMNMLLEANCATDGS
ANSRYYMAARPFFTNTAVNVDDKNTTAILEYTDAPPSAGPPDSPDLPAMDDIAAATAYTA
QLRSLVTKEHPIDVPMEVDEHMLVTISVNTIPCEPNKTCAGPGNNRLAASLNNVSFMNPT
IDILDAYYDSISGVYEPDFPNKPPFFFNFTAPNPPQDLWFTKRGTKVKVVEYGTVLEVVF
QDTAILGAESHPMHLHGFSFYVVGRGFGNFDKDKDPATYNLVDPPYQNTVSVPTGGWAAM
RFRAANPGVWFMHCHFDRHTVWGMDTVFIVKNGKGPDAQMMPRPPNMPKC
SEQ ID NO: 2 Laccase 3 genomic nucleic acid
ACCGACTGGTGGCGGCATGACGAACGAAACATGCATATGCATTCGTCCCCTCGTC
GTCGTTGGCAGCTCTCGCTCCTCTATAAATACCAGCGCCATCCGCTTCAGATGAG
CATCGATCCCAGCAACGCACGGAGCGTACGTACATTGCAGTAGCTAGCTATAGCT
GGCCGGCCATCCCCTCTCGCTCGCTGCTAAACACGTTCCAGCTTGTTTGCTCAGA
GAAACAGCGCGCGCGCACACACACACACATCATCATCATCGATTCATCGTACACA
GGATCAGAGAGCTTAATTAGTTCTAGCTCTGCTGCATGCCCCTTCGACAACGTCC
GACGATGGGCGGCGGCGGCGGCGGTGTAGCTAAGATGCCGGCAGGCCAGCTCT
GGTTATTACTGCTAGGCGTGTTGTTGTTAGCATTTGGAGTCCCAGCCCAGGCCTC
CAGGAATACTCACTACGACTTCGTTGTAAGTTGATCGATCTCGACGATATATCTCA
ATATCTCATTTCGACTTTATATTACCGGCCAATTTAATTTCAGAGACGCAGGCAGTT
ACCTGAGAACGTTAGTGCTAGCTTGCGGGCACCTTACAAATAATCAGCCGCTGTA
CATGCGTGTTCTAGTTTTCAAATAAATGTCTAGCTACTAGGTTGATGTGATTCATGT
TTGCATGGCTTCAGAACATTTGAATTCAAGTGTCTAGCTAGGTCTAGGTACGTAGT
CTATCTGATTGGTTGCCCGTATCGCTTTATTCTGGTCCTCTGCGTGCATTGACTTT
GATTAAGCGTGTGTTTGGTTACCCGCACGTGGAAGGTGCGGATTGTACGAGCCG
ATGCAAAACAAGCCATTTAGAGGCCAATTGACTGCATCCGCTCGTACCGCCGAAA
TGCCGGTATCTGGCCAGCCAGGCTCAGGACAGCCTGTAGCTACGGACACACTTA
ACCAATCAGGGCCAGTATGAACCGTTGGATGCTCTGCATGAGCATGATGGAGTAA
GTAGAACTAGCTAGCTGTACTGTACTGTATCCCGACAGGCACAGCCACAGATGAT
TGACTTCTTACTTGCTTGTACGTCTAGCTATAAACCGTATTAATTAGTTAGGTATAT
TTGCTAAGCATAGAAACTACGGGAAACCACACTTTTCCCGTTTCCCCATCATCGTC
ATCACTTGAGAAAAAAAATCTAGCAACGATGTGACACGTCTTATTAGATAGCGCTG
CTTGTTAATCTTTTTCCATTCCAGCTATCTAGCTATGCTCCTTGACGCGCACTGCTA
CGTGCTACAATATATACATACATACATAATATATGTATGTGTATACATGATTTTAATA
TATGTAGAGAAGGAGGACGACGACGTCTAAATATAACGCCGGTGTGGGGTGTGT
GATATATATATATTTTGCAGATAACTGAGACGAAGGTCACCCGACTATGCCATGAG
AAGACCATCCTGGCCGTGAACGGGCAGTTCCCGGGGCCGACCATCTACGCGCGC
AAGGACGACGTGGTCATCGTCAACGTGTACAACCAGGGCTACAAGAACATCACCC
TCCACTGGCACGGCGTGGACCAGCCGCGGAACCCGTGGTCCGATGGCCCGGAG
TACATCACGCAGTGCCCCATCCAGCCCGGCGCCAACTTCACCTACAAGATCATCT
TCACCGAGGAGGAAGGCACGCTGTGGTGGCACGCGCACAGCGAATTCGACCGC
GCCACCGTGCACGGCGCCATCGTCATCCACCCCAAGCGCGGCACCGTCTACCCC
TACCCCAAGCCGCACAAGGAGATGCCCATCATCCTCGGCGAGTGGTGGAACGCG
GACGTGGAGCAGATCCTCCTCGAGTCCCAGCGGACCGGCGGCGACGTCAACATT
TCGGACGCCAACACCATCAACGGCCAGCCCGGCGACTTCGCCCCGTGCTCCAAG
GAGGACACCTTCAAGATGTCCGTGGAGCACGGCAAGACGTACCTGCTCCGGGTC
ATCAACGCGGGGCTCACCAACGAGATGTTCTTCGCCGTCGCCGGGCACCGCCTC
ACGGTGGTCGGCACCGACGGCCGCTACCTCAGGCCGTTCACCGTCGACTACATC
CTCATCTCCCCCGGACAGACCATGAACATGCTCCTCGAGGCCAACTGCGCCACC
GACGGCTCAGCCAACAGCCGCTACTACATGGCTGCGAGGCCGTTCTTCACCAAC
ACGGCAGTCAATGTCGACGACAAAAACACCACGGCCATTCTGGAGTACACGGAC
GCGCCACCCTCCGCGGGGCCACCGGACTCCCCCGACCTGCCGGCCATGGACGA
CATCGCCGCGGCGACGGCGTACACGGCGCAGCTCCGGTCCCTGGTCACCAAGG
AGCATCCGATCGACGTGCCGATGGAGGTGGACGAGCACATGCTCGTGACGATCT
CCGTCAACACGATCCCCTGCGAGCCCAACAAGACGTGCGCCGGCCCCGGAAACA
ACCGCCTCGCCGCGAGCCTGAACAACGTCAGCTTCATGAACCCGACCATCGACA
TCCTCGACGCCTACTACGACTCCATCAGCGGCGTGTACGAGCCGGACTTCCCCA
ACAAGCCGCCCTTCTTCTTCAACTTCACCGCTCCCAACCCGCCACAGGACCTCTG
GTTCACGAAGCGGGGCACCAAGGTGAAGGTGGTGGAGTACGGCACCGTCCTGG
AGGTGGTGTTCCAGGACACGGCCATCCTCGGCGCCGAGAGCCACCCCATGCACC
TGCACGGCTTCAGCTTCTACGTGGTGGGCCGAGGCTTCGGTAACTTCGACAAGG
ACAAGGACCCCGCCACGTACAACCTGGTCGACCCGCCGTACCAGAACACCGTCT
CCGTGCCCACGGGCGGTTGGGCTGCAATGCGCTTCCGAGCGGCAAATCCTGGTG
AGTACTTTATTTACTAAATTAATTAAAATTTGGGCAACAATATATATATATATATATAT
ATATATATTGTTCTATGGATTTTATTTTCTTATATATGTGTGCTGTTTTTTCTTATATT
TTTTTAGGTGTGTGGTTTATGCATTGCCACTTTGATCGTCACACGGTGTGGGGCAT
GGACACTGTGTTCATTGTGAAAAATGGCAAGGGCCCGGACGCTCAGATGATGCCA
CGTCCCCCTAACATGCCCAAGTGCTGAGAAAACAAGGGCACGAGCTACGACTGC
TCGGGTTGCATGCAAGGCGCTCGATCAAACCAGCTAATCTTAGTTGATTGGTTGA
TTTAATTATTTGTGGTACATATTTTAAGTAGAACGGTTCTTCAAATAAAACGGCCAG
TTGAGATGTAATTAGTGTCATTTGTGTTCTTTTCTCTTTTTATTCATTTGATTGTAAG
AGAAAAACAAATTCATTATATTTATTATTTGTGTCGGTCTACTGCTAGTTCAATCTC
CAAGTGTAATTAAACAATGTATGTCAAATCATGTATCTAGTGAAAATTCAATATAAA
TGCGTGCTTCATATGTGTATTTATTT
SEQ ID NO: 3 Laccase 3 CDS nucleic acid
gcaggccagc tctggttatt actgctaggc gtgttgttgt tagcatttgg agtcccagcc
caggcctcca ggaatactca ctacgacttc gttataactg agacgaaggt cacccgacta
tgccatgaga agaccatcct ggccgtgaac gggcagttcc cggggccgac catctacgcg
cgcaaggacg acgtggtcat cgtcaacgtg tacaaccagg gctacaagaa catcaccctc
cactggcacg gcgtggacca gccgcggaac ccgtggtccg atggcccgga gtacatcacg
cagtgcccca tccagcccgg cgccaacttc acctacaaga tcatcttcac cgaggaggaa
ggcacgctgt ggtggcacgc gcacagcgaa ttcgaccgcg ccaccgtgca cggcgccatc
gtcatccacc ccaagcgcgg caccgtctac ccctacccca agccgcacaa ggagatgccc
atcatcctcg gcgagtggtg gaacgcggac gtggagcaga tcctcctcga gtcccagcgg
accggcggcg acgtcaacat ttcggacgcc aacaccatca acggccagcc cggcgacttc
gccccgtgct ccaaggagga caccttcaag atgtccgtgg agcacggcaa gacgtacctg
ctccgggtca tcaacgcggg gctcaccaac gagatgttct tcgccgtcgc cgggcaccgc
ctcacggtgg tcggcaccga cggccgctac ctcaggccgt tcaccgtcga ctacatcctc
atctcccccg gacagaccat gaacatgctc ctcgaggcca actgcgccac cgacggctca
gccaacagcc gctactacat ggctgcgagg ccgttcttca ccaacacggc agtcaatgtc
gacgacaaaa acaccacggc cattctggag tacacggacg cgccaccctc cgcggggcca
ccggactccc ccgacctgcc ggccatggac gacatcgccg cggcgacggc gtacacggcg
cagctccggt ccctggtcac caaggagcat ccgatcgacg tgccgatgga ggtggacgag
cacatgctcg tgacgatctc cgtcaacacg atcccctgcg agcccaacaa gacgtgcgcc
ggccccggaa acaaccgcct cgccgcgagc ctgaacaacg tcagcttcat gaacccgacc
atcgacatcc tcgacgccta ctacgactcc atcagcggcg tgtacgagcc ggacttcccc
aacaagccgc ccttcttctt caacttcacc gctcccaacc cgccacagga cctctggttc
acgaagcggg gcaccaaggt gaaggtggtg gagtacggca ccgtcctgga ggtggtgttc
caggacacgg ccatcctcgg cgccgagagc caccccatgc acctgcacgg cttcagcttc
tacgtggtgg gccgaggctt cggtaacttc gacaaggaca aggaccccgc cacgtacaac
ctggtcgacc cgccgtacca gaacaccgtc tccgtgccca cgggcggttg ggctgcaatg
cgcttccgag cggcaaatcc tggtgtgtgg tttatgcatt gccactttga tcgtcacacg
gtgtggggca tggacactgt gttcattgtg aaaaatggca agggcccgga cgctcagatg
atgccacgtc cccctaacat gcccaagtgc tga
SEQ ID NO: 4 Laccase 5 amino acid
MGARRGLRRGQAAAAAFSACPFLALAVVLLALPELAAGDTHYYTFNVQMTNVTRLCV
TKSIPTVNGEFPGPKLVVREGDRLVVKVHNHINYNVSFHWHGVRQLRNGWADGPSYI
TQCPIQGGQSYVYDFTVTGQRGTLWWHAHFSWLRVHLYGPLVILPKRGEGYPFPRP
YKEVPILFGEWFNADTEAVINQALQTGAGPNVSDAYTFNGLPGPTYNCSSKDTYKLKV
KPGRTYMLRLINSALNDELFFGIANHTLTVVEADASYVKPFTVSTLVISPGQTMNVLLTT
APSPASPAYAMAIAPYTNTQGTFDNTTAAAVLEYAPTTTRNNTLPPLPALPLYNDTGA
VSNFSRNFRSLNSARYPARVPVAVDRHLLFTVGLGTDPCPYTNQTCQGPNGTKFAAS
VNNNSFFRPRTALLEAHYRRRYAGVLLADFPTAPPHPFNYTGTPPNNTFVQHGTRVV
PLRFNASVELVLQGTSIQGAESHPLHLHGYNFFVVGQGFGNFDPVNDPPGYNLADPV
ERNTISVPTAGWVAVRFLADNPGVWLMHCHFDVHLSWGLSMAWLVNDGPLPNEKM
LPPPSDLPKC
SEQ ID NO: 5 Laccase 5 genomic nucleic acid (GRMZM2G367668)
CACCAACTAGGCCAACCACCACCGTGCTGTGACCCCCTACCATGCAGGCCACGA
ACCCGGCGGCCATCATGGCGCCTATATAGAACCCAGCACTCATTCCATAGCAAAG
TGCACCACTTCACTTGCTTCAAAGCGCAAACACACAAGAAGGGCGGAGCTGTTGT
CATCCTGACAATGGGCGCGCGTCGTGGTCTCCGGCGAGGCCAAGCCGCCGCCG
CCGCCTTCTCCGCATGTCCCTTCCTCGCCCTCGCCGTCGTCCTCCTCGCCTTGCC
GGAGCTCGCAGCCGGCGACACCCACTACTACACGTTCAACGTAAAACTACTCTAG
CGCACGAGCATTACAAGATCCAGCGCGCACACACACACATCGTCATCATCATCAT
CAGTGTTTCTCACTATTACTGGCCGGTATGGATTTGGTTCGTGCTGCAGGTGCAA
ATGACCAACGTGACACGGCTGTGCGTGACTAAGAGCATCCCGACGGTGAACGGG
GAGTTCCCGGGGCCGAAGCTGGTCGTGCGGGAAGGCGACCGCCTCGTGGTCAA
GGTTCACAACCACATCAACTACAATGTCTCGTTCCACTGGTAAGTGGGATCGTATC
ACAGCAGCAAGCGCACCGCGCCGTGTTGCCATCTCTCTGCCAGCCGGCCGGCGT
CTCATCACCCGCGTCGTCACGCAGGCACGGCGTCCGGCAGCTGCGCAACGGGT
GGGCGGACGGGCCGTCGTACATCACGCAGTGCCCGATCCAGGGCGGGCAGAGC
TACGTGTACGACTTCACCGTCACGGGGCAGCGCGGCACGCTGTGGTGGCACGC
GCACTTCTCCTGGCTGCGCGTGCACCTCTACGGCCCGCTCGTCATCCTCCCCAA
GCGCGGCGAGGGCTACCCGTTCCCGCGCCCCTACAAGGAGGTGCCCATCCTCTT
CGGTACAACGCCTGCCCGCTGCTCGCGCTCGTCGTCTTCCTTCCATCGATCGCTT
GATGGAGTTCCGTGCGTATTGCGTGGATTCTTTATTTGCAGGCGAATGGTTCAAC
GCGGACACGGAGGCCGTCATCAACCAGGCCCTGCAAACAGGCGCCGGCCCAAA
CGTCTCCGATGCCTACACCTTCAATGGGCTTCCAGGCCCGACATATAACTGCTCG
TCTAAAGGTACGCTTTCTTTGCAAGGGCTAGATTATTTAGGCCCATCGCTTGTACT
TTTTATTTAGTCATGTTATTAGAAAGATAAAACGAGATGAAGATAACAAAAGCCTGG
ATTTGTGATTTCTTTTTGGATGAAACAAAAAACCAGACACGTACAAGCTGAAGGTG
AAGCCCGGGAGGACGTACATGCTCCGGCTCATCAACTCCGCCCTCAACGACGAG
CTCTTCTTCGGCATCGCCAACCACACGCTCACCGTCGTCGAGGCGGACGCCAGC
TACGTCAAGCCATTCACCGTCAGCACGCTCGTCATTTCACCGGGGCAGACCATGA
ACGTGCTCCTCACGACGGCCCCCAGCCCCGCCTCCCCGGCCTACGCCATGGCG
ATCGCGCCCTACACCAACACGCAGGGCACGTTCGACAACACCACCGCCGCGGCC
GTCCTCGAGTACGCCCCGACGACGACCAGGAACAACACCCTGCCTCCCCTACCG
GCCCTGCCGCTGTACAACGACACCGGCGCGGTGTCCAACTTCTCGCGCAATTTC
CGCAGCCTGAACAGCGCGCGCTACCCGGCGCGCGTGCCGGTGGCGGTGGACCG
GCACCTGCTGTTCACCGTGGGGCTCGGCACGGACCCGTGCCCGTACACCAACCA
GACGTGCCAGGGCCCCAACGGCACCAAGTTCGCGGCGTCCGTCAACAACAACTC
CTTCTTCCGCCCCCGGACCGCGCTCCTCGAGGCGCACTACCGGCGCCGCTACGC
CGGCGTGCTCCTGGCCGACTTCCCCACGGCCCCGCCGCACCCGTTCAACTACAC
GGGCACCCCGCCCAACAACACGTTCGTGCAGCACGGCACGCGGGTGGTGCCGC
TCCGCTTCAACGCCTCCGTGGAGCTGGTGCTGCAGGGCACCAGCATCCAGGGCG
CCGAGAGCCACCCGCTGCACCTGCACGGCTACAACTTCTTCGTGGTCGGCCAAG
GGTTCGGCAACTTCGACCCGGTGAACGACCCGCCCGGGTACAACCTCGCCGACC
CCGTAGAGCGCAACACCATCAGCGTGCCCACCGCCGGCTGGGTCGCCGTCCGG
TTCCTCGCCGACAACCCGGGTAATCAATCAACTGATCCATGCACATTTATCATGCT
CTGCCTGTGTTGACTTGGTTGCATATATCTGGCAGGCGTGTGGCTGATGCATTGC
CACTTCGACGTGCACTTGAGCTGGGGCCTGTCCATGGCGTGGCTTGTCAACGAC
GGCCCGCTGCCGAACGAGAAGATGTTGCCCCCGCCATCCGACCTCCCAAAATGC
TGATGACGACTGGTCGTTTATCACCCGATCGAGGGGTAGATGGGCATTTAGGAAG
GTTCTCCTGCTTCCTGCACGTCTGCCTACTTCCTTTCCTTACGATGTTTGGAACTA
TTTGGTTTGGACTATTTAATTACCGTGTGCCGATTTTTGGCGAGTGCTTGGATTTC
GCGATCCTCGCTGAATCCCCTTTTGAAACATGTTAATCTGTATCTATGTAACGACA
ACGTTTGTTCTGCGGTTACTTGTTCTTTTTTTACCCCCTTTCTGAACATTCAGCACG
CATTGGTGTATTCACATGGTCAAATACAATGTAACAATGATGTCTGTAT
SEQ ID NO: 6 Laccase 5 CDS nucleic acid
ATGGGCGCGCGTCGTGGTCTCCGGCGAGGCCAAGCCGCCGCCGCCGCCTTCTC
CGCATGTCCCTTCCTCGCCCTCGCCGTCGTCCTCCTCGCCTTGCCGGAGCTCGC
AGCCGGCGACACCCACTACTACACGTTCAACGTGCAAATGACCAACGTGACACG
GCTGTGCGTGACTAAGAGCATCCCGACGGTGAACGGGGAGTTCCCGGGGCCGA
AGCTGGTCGTGCGGGAAGGCGACCGCCTCGTGGTCAAGGTTCACAACCACATCA
ACTACAATGTCTCGTTCCACTGGCACGGCGTCCGGCAGCTGCGCAACGGGTGGG
CGGACGGGCCGTCGTACATCACGCAGTGCCCGATCCAGGGCGGGCAGAGCTAC
GTGTACGACTTCACCGTCACGGGGCAGCGCGGCACGCTGTGGTGGCACGCGCA
CTTCTCCTGGCTGCGCGTGCACCTCTACGGCCCGCTCGTCATCCTCCCCAAGCG
CGGCGAGGGCTACCCGTTCCCGCGCCCCTACAAGGAGGTGCCCATCCTCTTCGG
CGAATGGTTCAACGCGGACACGGAGGCCGTCATCAACCAGGCCCTGCAAACAGG
CGCCGGCCCAAACGTCTCCGATGCCTACACCTTCAATGGGCTTCCAGGCCCGAC
ATATAACTGCTCGTCTAAAGACACGTACAAGCTGAAGGTGAAGCCCGGGAGGACG
TACATGCTCCGGCTCATCAACTCCGCCCTCAACGACGAGCTCTTCTTCGGCATCG
CCAACCACACGCTCACCGTCGTCGAGGCGGACGCCAGCTACGTCAAGCCATTCA
CCGTCAGCACGCTCGTCATTTCACCGGGGCAGACCATGAACGTGCTCCTCACGA
CGGCCCCCAGCCCCGCCTCCCCGGCCTACGCCATGGCGATCGCGCCCTACACC
AACACGCAGGGCACGTTCGACAACACCACCGCCGCGGCCGTCCTCGAGTACGCC
CCGACGACGACCAGGAACAACACCCTGCCTCCCCTACCGGCCCTGCCGCTGTAC
AACGACACCGGCGCGGTGTCCAACTTCTCGCGCAATTTCCGCAGCCTGAACAGC
GCGCGCTACCCGGCGCGCGTGCCGGTGGCGGTGGACCGGCACCTGCTGTTCAC
CGTGGGGCTCGGCACGGACCCGTGCCCGTACACCAACCAGACGTGCCAGGGCC
CCAACGGCACCAAGTTCGCGGCGTCCGTCAACAACAACTCCTTCTTCCGCCCCC
GGACCGCGCTCCTCGAGGCGCACTACCGGCGCCGCTACGCCGGCGTGCTCCTG
GCCGACTTCCCCACGGCCCCGCCGCACCCGTTCAACTACACGGGCACCCCGCCC
AACAACACGTTCGTGCAGCACGGCACGCGGGTGGTGCCGCTCCGCTTCAACGCC
TCCGTGGAGCTGGTGCTGCAGGGCACCAGCATCCAGGGCGCCGAGAGCCACCC
GCTGCACCTGCACGGCTACAACTTCTTCGTGGTCGGCCAAGGGTTCGGCAACTTC
GACCCGGTGAACGACCCGCCCGGGTACAACCTCGCCGACCCCGTAGAGCGCAA
CACCATCAGCGTGCCCACCGCCGGCTGGGTCGCCGTCCGGTTCCTCGCCGACAA
CCCGGGCGTGTGGCTGATGCATTGCCACTTCGACGTGCACTTGAGCTGGGGCCT
GTCCATGGCGTGGCTTGTCAACGACGGCCCGCTGCCGAACGAGAAGATGTTGCC
CCCGCCATCCGACCTCCCAAAATGCTGA
SEQ ID NO: 7 STTM nucleic acid sequence
aagaaaaatg gccatcccct agctaggtga agaagaatga aaacctctaa tttatctaga
ggttattcat cttttagggg atggcctaaa tacaaaatga aaactctcta attaagtggt
tttgtgttca tgtaaggaaa gcgttttaag atatggagca atgaagactg cagaaggctg
attcagactg cgagttttgt ttatctccct ctagaaactc ctctgcatct agccccttcc
aagcttcggt tcccctcgga atcagcagat tatgtatctt taattttgta atactctctc
tcttctctat gctttgtttt tcttcattat gtttgggttg tacccactcc cgcgcgttgt
gtgttctttg tgtgaggaat aaaaaaatat tcggatttga gaactaaaac tagagtagtt
ttattgatat tcttgttttt catttagtat ctaataagtt tggagaatag tcagaccagt
gcatgtaaat ttgcttccga ttctctttat agtgaattcc tctt
SEQ ID NO: 8 STTM RNA sequence
aagaaaaaug gccauccccu agcuagguga agaagaauga aaaccucuaa uuuaucuaga
gguuauucau cuuuuagggg auggccuaaa uacaaaauga aaacucucua auuaaguggu
uuuguguuca uguaaggaaa gcguuuuaag auauggagca augaagacug cagaaggcug
auucagacug cgaguuuugu uuaucucccu cuagaaacuc cucugcaucu agccccuucc
aagcuucggu uccccucgga aucagcagau uauguaucuu uaauuuugua auacucucuc
ucuucucuau gcuuuguuuu ucuucauuau guuuggguug uacccacucc cgcgcguugu
guguucuuug ugugaggaau aaaaaaauau ucggauuuga gaacuaaaac uagaguaguu
uuauugauau ucuuguuuuu cauuuaguau cuaauaaguu uggagaauag ucagaccagu
gcauguaaau uugcuuccga uucucuuuau agugaauucc ucuu
SEQ ID NO: 9: miR528 RNA sequence
UGGAAGGGGCAUGCAGAGGAG
SEQ ID NO: 10 miR528 nucleic acid sequence
TGGAAGGGGCATGCAGAGGAG
SEQ ID NO: 11: pCUB vector sequence

| | |
|---|---|
| ctcattaggc accccaggct ttacacttta tgcttccggc tcgtatgttg tgtggaattg | 60 |
| tgagcggata acaatttcac acaggaaaca gctatgacat gattacgaat tcccgatcta | 120 |
| gtaacataga tgacaccgcg cgcgataatt tatcctagtt tgcgcgctat attttgtttt | 180 |
| ctatcgcgta ttaaatgtat aattgcggga ctctaatcat aaaaacccat ctcataaata | 240 |
| acgtcatgca ttacatgtta attattacat gcttaacgta attcaacaga aattatatga | 300 |
| taatcatcgc aagaccggca acaggattca atcttaagaa actttattgc caaatgtttg | 360 |
| aacgatcggg gaaattcgag ctcggtaccc ggggatcctc tagagtcgac ctgcagaagt | 420 |
| aacaccaaac aacagggtga gcatcgacaa aagaaacagt accaagcaaa taaatagcgt | 480 |
| atgaaggcag ggctaaaaaa atccacatat agctgctgca tatgccatca tccaagtata | 540 |
| tcaagatcaa aataattata aaacatactt gtttattata atagataggt actcaaggtt | 600 |
| agagcatatg aatagatgct gcatatgcca tcatgtatat gcatcagtaa aacccacatc | 660 |
| aacatgtata cctatcctag atcgatattt ccatccatct taaactcgta actatgaaga | 720 |
| tgtatgacac acacatacag ttccaaaatt aataaataca ccaggtagtt tgaaacagta | 780 |
| ttctactccg atctagaacg aatgaacgac cgcccaacca caccacatca tcacaaccaa | 840 |
| gcgaacaaaa agcatctctg tatatgcatc agtaaaaccc gcatcaacat gtatacctat | 900 |
| cctagatcga tatttccatc catcatcttc aattcgtaac tatgaatatg tatggcacac | 960 |
| acatacagat ccaaaattaa taaatccacc aggtagtttg aaacagaatt aattctactc | 1020 |
| cgatctagaa cgaccgccca accagaccac atcatcacaa ccaagacaaa aaaaagcatg | 1080 |
| aaaagatgac ccgacaaaca agtgcacggc atatattgaa ataaaggaaa agggcaaacc | 1140 |
| aaaccctatg caacgaaaca aaaaaaatca tgaaatcgat cccgtctgcg gaacggctag | 1200 |
| agccatccca ggattcccca aagagaaaca ctggcaagtt agcaatcaga acgtgtctga | 1260 |
| cgtacaggtc gcatccgtgt acgaacgcta gcagcacgga tctaacacaa acacggatct | 1320 |
| aacacaaaca tgaacagaag tagaactacc gggccctaac catggaccgg aacgccgatc | 1380 |
| tagagaaggt agagaggggg ggggggggag gacgagcggc gtaccttgaa gcggaggtgc | 1440 |
| cgacgggtgg atttggggga gatctggttg tgtgtgtgtg cgctccgaac aacacgaggt | 1500 |
| tggggaaaga gggtgtggag ggggtgtcta tttattacgg cgggcgagga agggaaagcg | 1560 |
| aaggagcggt gggaaaggaa tcccccgtag ctgccggtgc cgtgagagga ggaggaggcc | 1620 |
| gcctgccgtg ccggctcacg tctgccgctc cgccacgcaa tttctggatg ccgacagcgg | 1680 |
| agcaagtcca acggtggagc ggaactctcg agaggggtcc agaggcagcg acagagatgc | 1740 |
| cgtgccgtct gcttcgcttg gcccgacgcg acgctgctgg ttcgctggtt ggtgtccgtt | 1800 |
| agactcgtcg acggcgttta acaggctggc attatctact cgaaacaaga aaaatgtttc | 1860 |
| cttagttttt ttaatttctt aaagggtatt tgtttaattt ttagtcactt tattttattc | 1920 |
| tattttatat ctaaattatt aaataaaaaa actaaaatag agttttagtt ttcttaattt | 1980 |
| agaggctaaa atagaataaa atagatgtac taaaaaaatt agtctataaa aaccattaac | 2040 |
| cctaaaccct aaatggatgt actaataaaa tggatgaagt attatatagg tgaagctatt | 2100 |
| tgcaaaaaaa aaggagaaca catgcacact aaaaagataa aactgtagag tcctgttgtc | 2160 |
| aaaatactca attgtccttt agaccatgtc taactgttca tttatatgat tctctaaaac | 2220 |
| actgatatta ttgtagtact atagattata ttattcgtag agtaaagttt aaatatatgt | 2280 |
| ataaagatag ataaactgca cttcaaacaa gtgtgacaaa aaaaatatgt ggtaattttt | 2340 |
| tataacttag acatgcaatg ctcattatct ctagagaggg cacgaccggg tcacgctgca | 2400 |
| ctgcaggcat gcaagcttgg cactggccgt cgttttacaa cgtcgtgact gggaaaaccc | 2460 |
| tggcgttacc caacttaatc gccttgcagc acatccccct ttcgccagct ggcgtaatag | 2520 |
| cgaagaggcc cgcaccgatc gcccttccca acagttgcgc agcctgaatg gcgaatgcta | 2580 |
| gagcagcttg agcttggatc agattgtcgt ttcccgcctt cagtttaaac tatcagtgtt | 2640 |
| tgacaggata tattggcggg taaacctaag agaaaagagc gtttattaga ataatcggat | 2700 |
| atttaaaagg gcgtgaaaag gtttatccgt tcgtccattt gtatgtgcat gccaaccaca | 2760 |
| gggttcccct cgggatcaaa gtactttgat ccaacccctc cgctgctata gtgcagtcgg | 2820 |
| cttctgacgt tcagtgcagc cgtcttctga aaacgacatg tcgcacaagt cctaagttac | 2880 |
| gcgacaggct gccgccctgc ccttttcctg gcgttttctt gtcgcgtgtt ttagtcgcat | 2940 |
| aaagtagaat acttgcgact agaaccggag acattacgcc atgaacaaga gcgccgccgc | 3000 |
| tggcctggtg ggctatgccc gcgtcagcac cgacgaccag gacttgacca accaacgggc | 3060 |
| cgaactgcac gcggcctatg aggtaaagag aaaatgagca aaagccaaac acgctaagtg | 3120 |
| ccggccgtcc gagcgcacgc agcagcaagg ctgcaacgtt ggccagcctg gcagacacgc | 3180 |
| cagccatgaa gcgggtcaac tttcagttgc cggagctggc caggatgctt gaccacctac | 3240 |
| gccctggcga cgttgtgaca gtgaccaggc tagaccgcct ggcccgcagc acccgcgacc | 3300 |
| tactggacat tgccgagcgc atccaggagg ccggcgcggg cctgcgtagc ctggcagagc | 3360 |
| cgtgggccga caccaccacg ccggccggcc gcatggtgtt gaccgtgttc gccggcattg | 3420 |
| ccgagttcga gcgttcccta atcatcgacc gcacccggag cgggcgcgag gccgccaagg | 3480 |
| cccgaggcgt gaagtttggc ccccgcccta ccctcacccc ggcacagatc gcgcacgccc | 3540 |
| gcgagctgat cgaccaggaa ggccgcaccg tgaaagaggc ggctgcactg cttggcgtgc | 3600 |
| atcgctcgac cctgtaccgc gcacttgagc gcagcgagga agtgacgccc accgaggcca | 3660 |
| ggcggcgcgg tgccttccgt gaggacgcat tgaccgaggc cgacgccctg gcggccgccg | 3720 |
| agaatgaacg ccaagaggaa caagcatgaa accgcaccag gacggccagg acgaaccgtt | 3780 |
| tttcattacc gaagagatcg aggcggagat gatcgcggcc gggtacgtgt tcgagccgcc | 3840 |
| cgcgcacgtc tcaaccgtgc ggctgcatga aatcctggcc ggtttgtctg atgccaagct | 3900 |
| ggcggcctgg ccggccagct tggccgctga agaaaccgag cgccgccgtc taaaaaggtg | 3960 |
| atgtgtattt gagtaaaaca gcttgcgtca tgcggtcgct gcgtatatga tgcgatgagt | 4020 |
| aaataaacaa atacgcaagg ggaacgcatg aaggttatcg ctgtacttaa ccagaaaggc | 4080 |
| gggtcaggca agacgaccat cgcaacccat ctagcccgcg ccctgcaact cgccggggcc | 4140 |
| gatgttctgt tagtcgattc cgatccccag ggcagtgccc gcgattgggc ggccgtgcgg | 4200 |
| gaagatcaac cgctaaccgt tgtcggcatc gaccgcccga cgattgaccg cgacgtgaag | 4260 |
| gccatcggcc ggcgcgactt cgtagtgatc gacggagcgc cccaggcggc ggacttggct | 4320 |
| gtgtccgcga tcaaggcagc cgacttcgtg ctgattccgg tgcagccaag cccttacgac | 4380 |
| atatgggcca ccgccgacct ggtggagctg gttaagcagc gcattgaggt cacggatgga | 4440 |
| aggctacaag cggcctttgt cgtgtcgcgg gcgatcaaag gcacgcgcat cggcggtgag | 4500 |
| gttgccgagg cgctggccgg gtacgagctg cccattcttg agtcccgtat cacgcagcgc | 4560 |
| gtgagctacc caggcactgc cgccgccggc acaaccgttc ttgaatcaga acccgagggc | 4620 |
| gacgctgccc gcgaggtcca ggcgctggcc gctgaaatta aatcaaaact catttgagtt | 4680 |
| aatgaggtaa agagaaaatg agcaaaagca caaacacgct aagtgccggc cgtccgagcg | 4740 |
| cacgcagcag caaggctgca acgttggcca gcctggcaga cacgccagcc atgaagcggg | 4800 |
| tcaactttca gttgccggcg gaggatcaca ccaagctgaa gatgtacgcg gtacgccaag | 4860 |
| gcaagaccat taccgagctg ctatctgaat acatcgcgca gctaccagag taaatgagca | 4920 |
| aatgaataaa tgagtagatg aattttagcg gctaaaggag gcggcatgga aaatcaagaa | 4980 |
| caaccaggca ccgacgccgt ggaatgcccc atgtgtggag gaacgggcgg ttggccaggc | 5040 |
| gtaagcggct gggttgcctg ccggccctgc aatggcactg gaacccccaa gcccgaggaa | 5100 |
| tcggcgtgag cggtcgcaaa ccatccggcc cggtacaaat cggcgcggcg ctgggtgatg | 5160 |
| acctggtgga gaagttgaag gccgcgcagg ccgcccagcg gcaacgcatc gaggcagaag | 5220 |
| cacgccccgg tgaatcgtgg caagcggccg ctgatcgaat ccgcaaagaa tcccggcaac | 5280 |
| cgccggcagc cggtgcgccg tcgattagga agccgcccaa gggcgacgag caaccagatt | 5340 |
| ttttcgttcc gatgctctat gacgtgggca cccgcgatag tcgcagcatc atggacgtgg | 5400 |
| ccgttttccg tctgtcgaag cgtgaccgac gagctggcga ggtgatccgc tacgagcttc | 5460 |
| cagacgggca cgtagaggtt tccgcagggc cggccggcat ggccagtgtg tgggattacg | 5520 |
| acctggtact gatggcggtt tcccatctaa ccgaatccat gaaccgatac cgggaaggga | 5580 |
| agggagacaa gcccggccgc gtgttccgtc cacacgttgc ggacgtactc aagttctgcc | 5640 |
| ggcgagccga tggcggaaag cagaaagacg acctggtaga aacctgcatt cggttaaaca | 5700 |
| ccacgcacgt tgccatgcag cgtacgaaga aggccaagaa cggccgcctg gtgacggtat | 5760 |
| ccgagggtga agccttgatt agccgctaca agatcgtaaa gagcgaaacc gggcggccgg | 5820 |
| agtacatcga gatcgagcta gctgattgga tgtaccgcga gatcacagaa ggcaagaacc | 5880 |
| cggacgtgct gacggttcac cccgattact ttttgatcga tcccggcatc ggccgttttc | 5940 |
| tctaccgcct ggcacgccgc gccgcaggca aggcagaagc cagatggttg ttcaagacga | 6000 |
| tctacgaacg cagtggcagc gccggagagt tcaagaagtt ctgtttcacc gtgcgcaagc | 6060 |
| tgatcgggtc aaatgacctg ccggagtacg atttgaagga ggaggcgggg caggctggcc | 6120 |
| cgatcctagt catgcgctac cgcaacctga tcgagggcga agcatccgcc ggttcctaat | 6180 |
| gtacggagca gatgctaggg caaattgccc tagcagggga aaaaggtcga aaaggtctct | 6240 |
| ttcctgtgga tagcacgtac attgggaacc caaagccgta cattgggaac cggaacccgt | 6300 |
| acattgggaa cccaaagccg tacattggga accggtcaca catgtaagtg actgatataa | 6360 |
| aagagaaaaa aggcgatttt tccgcctaaa actctttaaa acttattaaa actcttaaaa | 6420 |
| cccgcctggc ctgtgcataa ctgtctggcc agcgcacagc cgaagagctg caaaaagcgc | 6480 |
| ctacccttcg gtcgctgcgc tccctacgcc ccgccgcttc gcgtcggcct atcgcggccg | 6540 |
| ctggccgctc aaaaatggct ggcctacggc caggcaatct accagggcgc ggacaagccg | 6600 |
| cgccgtcgcc actcgaccgc cggcgcccac atcaaggcac cctgcctcgc gcgtttcggt | 6660 |
| gatgacggtg aaaacctctg acacatgcag ctcccggaga cggtcacagc ttgtctgtaa | 6720 |
| gcggatgccg ggagcagaca agcccgtcag ggcgcgtcag cgggtgttgg cgggtgtcgg | 6780 |
| ggcgcagcca tgacccagtc acgtagcgat agcggagtgt atactggctt aactatgcgg | 6840 |
| catcagagca gattgtactg agagtgcacc atatgcggtg tgaaataccg cacagatgcg | 6900 |
| taaggagaaa ataccgcatc aggcgctctt ccgcttcctc gctcactgac tcgctgcgct | 6960 |
| cggtcgttcg gctgcggcga gcggtatcag ctcactcaaa ggcggtaata cggttatcca | 7020 |
| cagaatcagg ggataacgca ggaaagaaca tgtgagcaaa aggccagcaa aaggccagga | 7080 |
| accgtaaaaa ggccgcgttg ctggcgtttt tccataggct ccgcccccct gacgagcatc | 7140 |
| acaaaaatcg acgctcaagt cagaggtggc gaaacccgac aggactataa agataccagg | 7200 |
| cgtttccccc tggaagctcc ctcgtgcgct ctcctgttcc gaccctgccg cttaccggat | 7260 |
| acctgtccgc ctttctccct tcgggaagcg tggcgctttc tcatagctca cgctgtaggt | 7320 |
| atctcagttc ggtgtaggtc gttcgctcca agctgggctg tgtgcacgaa ccccccgttc | 7380 |
| agcccgaccg ctgcgcctta tccggtaact atcgtcttga gtccaacccg gtaagacacg | 7440 |
| acttatcgcc actggcagca gccactggta acaggattag cagagcgagg tatgtaggcg | 7500 |
| gtgctacaga gttcttgaag tggtggccta actacggcta cactagaagg acagtatttg | 7560 |
| gtatctgcgc tctgctgaag ccagttacct tcggaaaaag agttggtagc tcttgatccg | 7620 |
| gcaaacaaac caccgctggt agcggtggtt tttttgtttg caagcagcag attacgcgca | 7680 |
| gaaaaaaagg atctcaagaa gatcctttga tcttttctac ggggtctgac gctcagtgga | 7740 |
| acgaaaactc acgttaaggg ctgatgaatc ccctaatgat tttggtaaaa atcattaagt | 7800 |
| taaggtggat acacatcttg tcatatgatc aaatggtttc gcgaaaaatc aataatcaga | 7860 |
| caacaagatg tgcgaactcg atattttaca cgactctctt taccaattct gccccgaatt | 7920 |
| acacttaaaa cgactcaaca gcttaacgtt ggcttgccac gcattacttg actgtaaaac | 7980 |
| tctcactctt accgaacttg gccgtaacct gccaaccaaa gcgagaacaa aacataacat | 8040 |
| caaacgaatc gaccgattgt taggtaatcg tcacctccac aaagagcgac tcgctgtata | 8100 |
| ccgttggcat gctagcttta tctgttcggg caatacgatg cccattgtac ttgttgactg | 8160 |
| gtctgatatt cgtgagcaaa aacgacttat ggtattgcga gcttcagtcg cactacacgg | 8220 |
| tcgttctgtt actctttatg agaaagcgtt cccgctttca gagcaatgtt caaagaaagc | 8280 |
| tcatgaccaa tttctagccg accttgcgag cattctaccg agtaacacca caccgctcat | 8340 |
| tgtcagtgat gctggcttta aagtgccatg gtataaatcc gttgagaagc tgggttggta | 8400 |
| ctggttaagt cgagtaagag gaaaagtaca atatgcagac ctaggagcgg aaaactggaa | 8460 |
| acctatcagc aacttacatg atatgtcatc tagtcactca aagactttag gctataagag | 8520 |
| gctgactaaa agcaatccaa tctcatgcca aattctattg tataaatctc gctctaaagg | 8580 |
| ccgaaaaaat cagcgctcga cacggactca ttgtcaccac ccgtcaccta aaatctactc | 8640 |
| agcgtcggca aaggagccat gggttctagc aactaactta cctgttgaaa ttcgaacacc | 8700 |
| caaacaactt gttaatatct attcgaagcg aatgcagatt gaagaaacct tccgagactt | 8760 |
| gaaaagtcct gcctacggac taggcctacg ccatagccga acgagcagct cagagcgttt | 8820 |
| tgatatcatg ctgctaatcg ccctgatgct tcaactaaca tgttggcttg cgggcgttca | 8880 |
| tgctcagaaa caaggttggg acaagcactt ccaggctaac acagtcagaa atcgaaacgt | 8940 |
| actctcaaca gttcgcttag gcatggaagt tttgcggcat tctggctaca caataacaag | 9000 |
| ggaagactta ctcgtggctg caaccctact agctcaaaat ttattcacac atggttacgc | 9060 |
| tttggggaaa ttatgagggg atctctcagc gttaagggat tttggtcatg cattctaggt | 9120 |
| actaaaacaa ttcatccagt aaaatataat attttatttt ctcccaatca ggcttgatcc | 9180 |
| ccagtaagtc aaaaaatagc tcgacatact gttcttcccc gatatcctcc ctgatcgacc | 9240 |
| ggacgcagaa ggcaatgtca taccacttgt ccgccctgcc gcttctccca agatcaataa | 9300 |
| agccacttac tttgccatct ttcacaaaga tgttgctgtc tcccaggtcg ccgtgggaaa | 9360 |
| agacaagttc ctcttcgggc ttttccgtct ttaaaaaatc atacagctcg cgcggatctt | 9420 |
| taaatggagt gtcttcttcc cagttttcgc aatccacatc ggccagatcg ttattcagta | 9480 |
| agtaatccaa ttcggctaag cggctgtcta agctattcgt atagggacaa tccgatatgt | 9540 |
| cgatggagtg aaagagcctg atgcactccg catacagctc gataatcttt tcagggcttt | 9600 |
| gttcatcttc atactcttcc gagcaaagga cgccatcggc ctcactcatg agcagattgc | 9660 |
| tccagccatc atgccgttca aagtgcagga cctttggaac aggcagcttt ccttccagcc | 9720 |
| atagcatcat gtccttttcc cgttccacat cataggtggt ccctttatac cggctgtccg | 9780 |
| tcatttttaa atataggttt tcattttctc ccaccagctt atatacctta gcaggagaca | 9840 |
| ttccttccgt atcttttacg cagcggtatt tttcgatcag ttttttcaat tccggtgata | 9900 |
| ttctcatttt agccatttat tatttccttc ctcttttcta cagtatttaa agatacccca | 9960 |
| agaagctaat tataacaaga cgaactccaa ttcactgttc cttgcattct aaaaccttaa | 10020 |
| ataccagaaa acagcttttt caaagttgtt ttcaaagttg gcgtataaca tagtatcgac | 10080 |
| ggagccgatt ttgaaaccgc ggtgatcaca ggcagcaacg ctctgtcatc gttacaatca | 10140 |
| acatgctacc ctccgcgaga tcatccgtgt ttcaaacccg gcagcttagt tgccgttctt | 10200 |
| ccgaatagca tcggtaacat gagcaaagtc tgccgcctta caacggctct cccgctgacg | 10260 |
| ccgtcccgga ctgatgggct gcctgtatcg agtggtgatt ttgtgccgag ctgccggtcg | 10320 |
| gggagctgtt ggctggctgg tggcaggata tattgtggtg taaacaaatt gacgcttaga | 10380 |
| caacttaata acacattgcg gacgttttta atgtactgaa ttaacgccga attaattcgg | 10440 |
| gggatctgga ttttagtact ggattttggt tttaggaatt agaaatttta ttgatagaag | 10500 |
| tattttacaa atacaaatac atactaaggg tttcttatat gctcaacaca tgagcgaaac | 10560 |
| cctataggaa ccctaattcc cttatctggg aactactcac acattattat ggagaaactc | 10620 |
| gagtcaaatc tcggtgacgg gcaggaccgg acggggcggt accggcaggc tgaagtccag | 10680 |
| ctgccagaaa cccacgtcat gccagttccc gtgcttgaag ccggccgccc gcagcatgcc | 10740 |
| gcggggggca tatccgagcg cctcgtgcat gcgcacgctc gggtcgttgg gcagcccgat | 10800 |
| gacagcgacc acgctcttga agccctgtgc ctccagggac ttcagcaggt gggtgtagag | 10860 |
| cgtggagccc agtcccgtcc gctggtggcg gggggagacg tacacggtcg actcggccgt | 10920 |
| ccagtcgtag gcgttgcgtg ccttccaggg gcccgcgtag gcgatgccgg cgacctcgcc | 10980 |
| gtccacctcg gcgacgagcc agggatagcg ctcccgcaga cggacgaggt cgtccgtcca | 11040 |
| ctcctgcggt tcctgcggct cggtacggaa gttgaccgtg cttgtctcga tgtagtggtt | 11100 |
| gacgatggtg cagaccgccg gcatgtccgc ctcggtggca cggcggatgt cggccgggcg | 11160 |
| tcgttctggg ctcatggtag actcgagaga gatagatttg tagagagaga ctggtgattt | 11220 |
| cagcgtgtcc tctccaaatg aaatgaactt ccttatatag aggaagggtc ttgcgaagga | 11280 |
| tagtgggatt gtgcgtcatc ccttacgtca gtggagatat cacatcaatc cacttgcttt | 11340 |
| gaagacgtgg ttggaacgtc ttctttttcc acgatgctcc tcgtgggtgg gggtccatct | 11400 |
| ttgggaccac tgtcggcaga ggcatcttga acgatagcct ttcctttatc gcaatgatgg | 11460 |
| catttgtagg tgccaccttc cttttctact gtccttttga tgaagtgaca gatagctggg | 11520 |
| caatggaatc cgaggaggtt tcccgatatt accctttgtt gaaaagtctc aatagccctt | 11580 |
| tggtcttctg agactgtatc tttgatattc ttggagtaga cgagagtgtc gtgctccacc | 11640 |
| atgttcacat caatccactt gctttgaaga cgtggttgga acgtcttctt tttccacgat | 11700 |
| gctcctcgtg ggtgggggtc catctttggg accactgtcg gcagaggcat cttgaacgat | 11760 |
| agcctttcct ttatcgcaat gatggcattt gtaggtgcca ccttcctttt ctactgtcct | 11820 |
| tttgatgaag tgacagatag ctgggcaatg gaatccgagg aggtttcccg atattaccct | 11880 |
| ttgttgaaaa gtctcaatag ccctttggtc ttctgagact gtatctttga tattcttgga | 11940 |
| gtagacgaga gtgtcgtgct ccaccatgtt ggcaagctgc tctagccaat acgcaaaccg | 12000 |
| cctctccccg cgcgttggcc gattcattaa tgcagctggc acgacaggtt tcccgactgg | 12060 |
| aaagcgggca gtgagcgcaa cgcaattaat gtgagttagc tca | 12103 |

SEQ ID NO: 12 (PCR amplification product)

| | |
|---|---|
| gactctagag gatccatgcc ccttcgacaa cgtccgacga tgggcggcgg cggcggcggt | 60 |
| gtagctaaga tgccggcagg ccagctctgg ttattactgc taggcgtgtt gttgttagca | 120 |
| tttggagtcc cagcccaggc ctccaggaat actcactacg acttcgttat aactgagacg | 180 |
| aaggtcaccc gactatgcca tgagaagacc atcctggccg tgaacgggca gttcccgggg | 240 |
| ccgaccatct acgcgcgcaa ggacgacgtg gtcatcgtca acgtgtacaa ccagggctac | 300 |
| aagaacatca ccctccactg gcacggcgtg gaccagccgc ggaacccgtg gtccgatggc | 360 |
| ccggagtaca tcacgcagtg ccccatccag cccggcgcca acttcaccta caagatcatc | 420 |
| ttcaccgagg aggaaggcac gctgtggtgg cacgcgcaca gcgaattcga ccgcgccacc | 480 |
| gtgcacggcg ccatcgtcat ccaccccaag cgcggcaccg tctaccccta ccccaagccg | 540 |
| cacaaggaga tgcccatcat cctcggcgag tggtggaacg cggacgtgga gcagatcctc | 600 |
| ctcgagtccc agcggaccgg cggcgacgtc aacatttcgg acgccaacac catcaacggc | 660 |
| cagcccggcg acttcgcccc gtgctccaag gaggacacct tcaagatgtc cgtggagcac | 720 |
| ggcaagacgt acctgctccg ggtcatcaac gcggggctca ccaacgagat gttcttcgcc | 780 |
| gtcgccgggc accgcctcac ggtggtcggc accgacggcc gctacctcag gccgttcacc | 840 |
| gtcgactaca tcctcatctc ccccggacag accatgaaca tgctcctcga ggccaactgc | 900 |
| gccaccgacg gctcagccaa cagccgctac tacatggctg cgaggccgtt cttcaccaac | 960 |
| acggcagtca atgtcgacga caaaaacacc acggccattc tggagtacac ggacgcgcca | 1020 |
| ccctccgcgg ggccaccgga ctcccccgac ctgccggcca tggacgacat cgccgcggcg | 1080 |
| acggcgtaca cggcgcagct ccggtccctg gtcaccaagg agcatccgat cgacgtgccg | 1140 |
| atggaggtgg acgagcacat gctcgtgacg atctccgtca acacgatccc ctgcgagccc | 1200 |
| aacaagacgt gcgccggccc cggaaacaac cgcctcgccg cgagcctgaa caacgtcagc | 1260 |
| ttcatgaacc cgaccatcga catcctcgac gcctactacg actccatcag cggcgtgtac | 1320 |
| gagccggact tccccaacaa gccgcccttc ttcttcaact tcaccgctcc caacccgcca | 1380 |
| caggacctct ggttcacgaa gcggggcacc aaggtgaagg tggtggagta cggcaccgtc | 1440 |
| ctggaggtgg tgttccagga cacggccatc ctcggcgccg agagccaccc catgcacctg | 1500 |
| cacggcttca gcttctacgt ggtgggccga ggcttcggta acttcgacaa ggacaaggac | 1560 |
| cccgccacgt acaacctggt cgacccgccg taccagaaca ccgtctccgt gcccacgggc | 1620 |
| ggttgggctg caatgcgctt ccgagcggca aatcctggtg tgtggtttat gcattgccac | 1680 |
| tttgatcgtc acacggtgtg gggcatggac actgtgttca ttgtgaaaaa tggcaagggc | 1740 |
| ccggacgctc agatgatgcc acgtccccct aacatgccca agtgctgagg atccccgggt | 1800 |
| acc | 1803 |

SEQ ID NO: 13 (nucleic acid sequenc of precursor of miR528)

| | |
|---|---|
| gtggaagggg catgcagagg agcacgaacg aggtgtggtt ggcccctcgt tagctctcct | 60 |
| gtgcctgcct cttccatt | 78 |

SEQ ID NO: 14 (precursor RNA of miR528)

| | |
|---|---|
| guggaagggg caugcagagg agcacgaacg aggugugguu ggccccucgu uagcucuccu | 60 |
| gugccugccu cuuccauu | 78 |

SEQ ID NO: 15 (miR528)

| | |
|---|---|
| uggaaggggc augcagagga g | 21 |

SEQ ID NO: 16 (corresponding DNA of GRMZM2G367668 transcript)

| | |
|---|---|
| caccaactag gccaaccacc accgtgctgt gaccccctac catgcaggcc acgaacccgg | 60 |
| cggccatcat ggcgcctata tagaacccag cactcattcc atagcaaagt gcaccacttc | 120 |
| acttgcttca aagcgcaaac acacaagaag ggcggagctg ttgtcatcct gacaatgggc | 180 |
| gcgcgtcgtg gtctccggcg aggccaagcc gccgccgccg ccttctccgc atgtcccttc | 240 |
| ctcgccctcg ccgtcgtcct cctcgccttg ccggagctcg cagccggcga cacccactac | 300 |
| tacacgttca acgtgcaaat gaccaacgtg acacggctgt gcgtgactaa gagcatcccg | 360 |
| acggtgaacg gggagttccc ggggccgaag ctggtcgtgc gggaaggcga ccgcctcgtg | 420 |
| gtcaaggttc acaaccacat caactacaat gtctcgttcc actggcacgg cgtccggcag | 480 |
| ctgcgcaacg ggtgggcgga cgggccgtcg tacatcacgc agtgcccgat ccagggcggg | 540 |
| cagagctacg tgtacgactt caccgtcacg gggcagcgcg gcacgctgtg gtggcacgcg | 600 |
| cacttctcct ggctgcgcgt gcacctctac ggcccgctcg tcatcctccc caagcgcggc | 660 |
| gagggctacc cgttcccgcg cccctacaag gaggtgccca tcctcttcgg cgaatggttc | 720 |
| aacgcggaca cggaggccgt catcaaccag gccctgcaaa caggcgccgg cccaaacgtc | 780 |
| tccgatgcct acaccttcaa tgggcttcca ggcccgacat ataactgctc gtctaaagac | 840 |
| acgtacaagc tgaaggtgaa gcccgggagg acgtacatgc tccggctcat caactccgcc | 900 |
| ctcaacgacg agctcttctt cggcatcgcc aaccacacgc tcaccgtcgt cgaggcggac | 960 |
| gccagctacg tcaagccatt caccgtcagc acgctcgtca tttcaccggg gcagaccatg | 1020 |
| aacgtgctcc tcacgacggc ccccagcccc gcctccccgg cctacgccat ggcgatcgcg | 1080 |
| ccctacacca acacgcaggg cacgttcgac aacaccaccg ccgcggccgt cctcgagtac | 1140 |
| gccccgacga cgaccaggaa caacaccctg cctcccctac cggccctgcc gctgtacaac | 1200 |
| gacaccggcg cggtgtccaa cttctcgcgc aatttccgca gcctgaacag cgcgcgctac | 1260 |
| ccggcgcgcg tgccggtggc ggtggaccgg cacctgctgt tcaccgtggg gctcggcacg | 1320 |
| gacccgtgcc cgtacaccaa ccagacgtgc cagggcccca acggcaccaa gttcgcggcg | 1380 |
| tccgtcaaca acaactcctt cttccgcccc cggaccgcgc tcctcgaggc gcactaccgg | 1440 |
| cgccgctacg ccggcgtgct cctggccgac ttccccacgg ccccgccgca cccgttcaac | 1500 |
| tacacgggca ccccgcccaa caacacgttc gtgcagcacg gcacgcgggt ggtgccgctc | 1560 |
| cgcttcaacg cctccgtgga gctggtgctg cagggcacca gcatccaggg cgccgagagc | 1620 |
| cacccgctgc acctgcacgg ctacaacttc ttcgtggtcg gccaagggtt cggcaacttc | 1680 |
| gacccggtga acgacccgcc cgggtacaac ctcgccgacc ccgtagagcg caacaccatc | 1740 |
| agcgtgccca ccgccggctg ggtcgccgtc cggttcctcg ccgacaaccc gggcgtgtgg | 1800 |
| ctgatgcatt gccacttcga cgtgcacttg agctggggcc tgtccatggc gtggcttgtc | 1860 |
| aacgacggcc cgctgccgaa cgagaagatg ttgcccccgc catccgacct cccaaaatgc | 1920 |
| tgatgacgac tggtcgttta tcacccgatc gaggggtaga tgggcattta ggaaggttct | 1980 |
| cctgcttcct gcacgtctgc ctacttcctt tccttacgat gtttggaact atttggtttg | 2040 |
| gactatttaa ttaccgtgtg ccgatttttg gcgagtgctt ggatttcgcg atcctcgctg | 2100 |
| aatccccttt tgaaacatgt taatctgtat ctatgtaacg acaacgtttg ttctgcggtt | 2160 |
| acttgttctt tttttacccc ctttctgaac attcagcacg cattggtgta ttcacatggt | 2220 |
| caaatacaat gtaacaatga tgtctgtat | 2249 |

SEQ ID NO: 17 (corresponding DNA of GRMZM2G169033 transcript)

| | |
|---|---|
| accgactggt ggcggcatga cgaacgaaac atgcatatgc attcgtcccc tcgtcgtcgt | 60 |
| tggcagctct cgctcctcta taaataccag cgccatccgc ttcagatgag catcgatccc | 120 |
| agcaacgcac ggagcgtacg tacattgcag tagctagcta tagctggccg gccatcccct | 180 |
| ctcgctcgct gctaaacacg ttccagcttg tttgctcaga gaaacagcgc gcgcgcacac | 240 |
| acacacacat catcatcatc gattcatcgt acacaggatc agagagctta attagttcta | 300 |
| gctctgctgc atgccccttc gacaacgtcc gacgatgggc ggcggcggcg gcggtgtagc | 360 |
| taagatgccg gcaggccagc tctggttatt actgctaggc gtgttgttgt tagcatttgg | 420 |
| agtcccagcc caggcctcca ggaatactca ctacgacttc gttataactg agacgaaggt | 480 |
| cacccgacta tgccatgaga agaccatcct ggccgtgaac gggcagttcc cggggccgac | 540 |
| catctacgcg cgcaaggacg acgtggtcat cgtcaacgtg tacaaccagg gctacaagaa | 600 |
| catcaccctc cactggcacg gcgtggacca gccgcggaac ccgtggtccg atggcccgga | 660 |
| gtacatcacg cagtgcccca tccagcccgg cgccaacttc acctacaaga tcatcttcac | 720 |
| cgaggaggaa ggcacgctgt ggtggcacgc gcacagcgaa ttcgaccgcg ccaccgtgca | 780 |
| cggcgccatc gtcatccacc ccaagcgcgg caccgtctac ccctacccca agccgcacaa | 840 |
| ggagatgccc atcatcctcg gcgagtggtg gaacgcggac gtggagcaga tcctcctcga | 900 |
| gtcccagcgg accggcggcg acgtcaacat ttcggacgcc aacaccatca acggccagcc | 960 |
| cggcgacttc gccccgtgct ccaaggagga caccttcaag atgtccgtgg agcacggcaa | 1020 |
| gacgtacctg ctccgggtca tcaacgcggg gctcaccaac gagatgttct tcgccgtcgc | 1080 |
| cgggcaccgc ctcacggtgg tcggcaccga cggccgctac ctcaggccgt tcaccgtcga | 1140 |
| ctacatcctc atctcccccg gacagaccat gaacatgctc ctcgaggcca actgcgccac | 1200 |
| cgacggctca gccaacagcc gctactacat ggctgcgagg ccgttcttca ccaacacggc | 1260 |
| agtcaatgtc gacgacaaaa acaccacggc cattctggag tacacggacg cgccaccctc | 1320 |
| cgcggggcca ccggactccc ccgacctgcc ggccatggac gacatcgccg cggcgacggc | 1380 |
| gtacacggcg cagctccggt ccctggtcac caaggagcat ccgatcgacg tgccgatgga | 1440 |
| ggtggacgag cacatgctcg tgacgatctc cgtcaacacg atcccctgcg agcccaacaa | 1500 |
| gacgtgcgcc ggccccggaa acaaccgcct cgccgcgagc ctgaacaacg tcagcttcat | 1560 |
| gaacccgacc atcgacatcc tcgacgccta ctacgactcc atcagcggcg tgtacgagcc | 1620 |
| ggacttcccc aacaagccgc ccttcttctt caacttcacc gctcccaacc cgccacagga | 1680 |
| cctctggttc acgaagcggg gcaccaaggt gaaggtggtg gagtacggca ccgtcctgga | 1740 |
| ggtggtgttc caggacacgg ccatcctcgg cgccgagagc caccccatgc acctgcacgg | 1800 |
| cttcagcttc tacgtggtgg gccgaggctt cggtaacttc gacaaggaca aggaccccgc | 1860 |
| cacgtacaac ctggtcgacc cgccgtacca gaacaccgtc tccgtgccca cgggcggttg | 1920 |
| ggctgcaatg cgcttccgag cggcaaatcc tggtgtgtgg tttatgcatt gccactttga | 1980 |
| tcgtcacacg gtgtggggca tggacactgt gttcattgtg aaaaatggca agggcccgga | 2040 |
| cgctcagatg atgccacgtc cccctaacat gcccaagtgc tgagaaaaca agggcacgag | 2100 |
| ctacgactgc tcgggttgca tgcaaggcgc tcgatcaaac cagctaatct tagttgattg | 2160 |
| gttgatttaa ttatttgtgg tacatatttt aagtagaacg gttcttcaaa taaaacggcc | 2220 |
| agttgagatg taattagtgt catttgtgtt cttttctctt tttattcatt tgattgtaag | 2280 |
| agaaaaacaa attcattata tttattattt gtgtcggtct actgctagtt caatctccaa | 2340 |
| gtgtaattaa acaatgtatg tcaaatcatg tatctagtga aaattcaata taaatgcgtg | 2400 |
| cttcatatgt gtatttattt | 2420 |

SEQ ID NO: 18 (probe for the GRMZM2G169033 transcript)

| | |
|---|---|
| cgctcgatca aaccagctaa tcttagttga ttggttgatt taattatttg tggtacatat | 60 |
| tttaagtaga acggttcttc aaataaaacg gccagttgag atgtaattag tgtcatttgt | 120 |
| gttcttttct ctttttattc atttgattgt aagagaaaaa caaattcatt atatttatta | 180 |
| tttgtgtcgg tctactgcta gttca | 205 |

SEQ ID NO: 19 (probe for the GRMZM2G367668 transcript)

| | |
|---|---|
| tttatcaccc gatcgagggg tagatgggca tttaggaagg ttctcctgct tcctgcacgt | 60 |
| ctgcctactt cctttcctta cgatgtttgg aactatttgg tttggactat ttaattaccg | 120 |
| tgtgccgatt tttggcgagt gcttggattt cgcgatcctc gctgaatccc c | 171 |

SEQ ID NO: 31 (tracrRNA)
GGGCGAAACAACACAGCGAGTTAAAATAAGGCTTAGTCCGTACTCAACTTGAAAA
GGTGGCACCGATTCGGTGTTTTT
SEQ ID NO: 32 (miR528a)
GTGGAAGGGGCATGCAGAGGAGGAGCACGAGCGAGGTGTGGCTGGAAGAAGCA
GCCGGGGCCTGTGTGCTATATACCCCTCGCAAGCTCTCCTCACTCCTCTCCTGTG
CCTGCCTCTTCCATT
SEQ ID NO: 33 (miR528a target 1)
TCAGGGTCAGTTTGCTCTGCTGG
SEQ ID NO: 34 (miR528a protospacer 1)
TCAGGGTCAGTTTGCTCTGC
SEQ ID NO: 35 (miR528a complete sgRNA 1)
TCAGGGTCAGTTTGCTCTGCGTTTTAGAGCTAGAAATAGCAAGTTAAAATAAGGCT
AGTCCGTTATCAACTTGAAAAAGTGGCACCGAGTCGGTGC
SEQ ID NO: 36 (miR528a target 2)
TGCGCAGTTGCCCTGTGATGAGG
SEQ ID NO: 37 (miR528a protospacer 2)
TGCGCAGTTGCCCTGTGATG
SEQ ID NO: 38 (mi528a complete sgRNA 2)
TGCGCAGTTGCCCTGTGATGGTTTTAGAGCTAGAAATAGCAAGTTAAAATAAGGCT
AGTCCGTTATCAACTTGAAAAAGTGGCACCGAGTCGGTGC
SEQ ID NO: 39 (miR528b)
GTGGAAGGGGCATGCAGAGGAGCACGAACGAGGTGTGGTTGGCCCCTCGTTAG
CTCTCCTGTGCCTGCCTCTTCCATT
SEQ ID NO: 40 (miR528b target 1)
TCTCTAGTTCTGGGAGTTCCTGG
SEQ ID NO: 41 (miR528b protospacer 1)
TCTCTAGTTCTGGGAGTTCC
SEQ ID NO: 42 (mi528b complete sg RNA 1)
TCTCTAGTTCTGGGAGTTCCGTTTTAGAGCTAGAAATAGCAAGTTAAAATAAGGCT
AGTCCGTTATCAACTTGAAAAAGTGGCACCGAGTCGGTGC
SEQ ID NO: 43 (miR528b target 2)
AAAACTCAGAAGCGCAACCGAGG
SEQ ID NO: 44 (miR528b protospacer 2)
AAAACTCAGAAGCGCAACCG
SEQ ID NO: 45 (miR528b complete sgRNA 2)
AAAACTCAGAAGCGCAACCGGTTTTAGAGCTAGAAATAGCAAGTTAAAATAAGGCT
AGTCCGTTATCAACTTGAAAAAGTGGCACCGAGTCGGTGC
SEQ ID NO: 46 (promoter sequence (ZmUbi) guiding Cas9
GTGCCCCTCT CTAGAGATAA TGAGCATTGC ATGTCTAAGT TATAAAAAAT
TACCACATAT TTTTTTTGTC ACACTTGTTT GAAGTGCAGT TTATCTATCT
TTATACATAT ATTTAAACTT TACTCTACGA ATAATATAAT CTATAGTACT
ACAATAATAT CAGTGTTTTA GAGAATCATA TAAATGAACA GTTAGACATG
GTCTAAAGGA CAATTGAGTA TTTTGACAAC AGGACTCTAC AGTTTTATCT
TTTTAGTGTG CATGTGTTCT CCTTTTTTTT TGCAAATAGC TTCACCTATA
TAATACTTCA TCCATTTTAT TAGTACATCC ATTTAGGGTT TAGGGTTAAT
GGTTTTTATA GACTAATTTT TTTAGTACAT CTATTTTATT CTATTTTAGC
CTCTAAATTA AGAAAACTAA AACTCTATTT TAGTTTTTTT ATTTAATAAT
TTAGATATAA AATAGAATAA AATAAAGTGA CTAAAAATTA AACAAATACC
CTTTAAGAAA TTAAAAAAAC TAAGGAAACA TTTTTCTTGT TTCGAGTAGA
TAATGCCAGC CTGTTAAACG CCGTCGACGA GTCTAACGGA CACCAACCAG
CGAACCAGCA GCGTCGCGTC GGGCCAAGCG AAGCAGACGG CACGGCATCT
CTGTCGCTGC CTCTGGACCC CTCTCGAGAG TTCCGCTCCA CCGTTGGACT
TGCTCCGCTG TCGGCATCCA GAAATTGCGT GGCGGAGCGG CAGACGTGAG
CCGGCACGGC AGGCGGCCTC CTCCTCCTCT CACGGCACCG GCAGCTACGG
GGGATTCCTT TCCCACCGCT CCTTCGCTTT CCCTTCCTCG CCCGCCGTAA
TAAATAGACA CCCCCTCCAC ACCCTCTTTC CCCAACCTCG TGTTGTTCGG
AGCGCACACA CACACAACCA GATCTCCCCC AAATCCACCC GTCGGCACCT
CCGCTTCAAG GTACGCCGCT CGTCCTCCCC CCCCCCCCCT CTCTACCTTC
TCTAGATCGG CGTTCCGGTC CATGGTTAGG GCCCGGTAGT TCTACTTCTG
TTCATGTTTG TGTTAGATCC GTGTTTGTGT TAGATCCGTG CTGCTAGCGT
TCGTACACGG ATGCGACCTG TACGTCAGAC ACGTTCTGAT TGCTAACTTG
CCAGTGTTTC TCTTTGGGGA ATCCTGGGAT GGCTCTAGCC GTTCCGCAGA
CGGGATCGAT TTCATGATTT TTTTTGTTTC GTTGCATAGG GTTTGGTTTG
CCCTTTTCCT TTATTTCAAT ATATGCCGTG CACTTGTTTG TCGGGTCATC
TTTTCATGCT TTTTTTTGTC TTGGTTGTGA TGATGTGGTC TGGTTGGGCG
GTCGTTCTAG ATCGGAGTAG AATTAATTCT GTTTCAAACT ACCTGGTGGA
TTTATTAATT TTGGATCTGT ATGTGTGTGC CATACATATT CATAGTTACG
AATTGAAGAT GATGGATGGA AATATCGATC TAGGATAGGT ATACATGTTG
ATGCGGGTTT TACTGATGCA TATACAGAGA TGCTTTTTGT TCGCTTGGTT
GTGATGATGT GGTGTGGTTG GGCGGTCGTT CATTCGTTCT AGATCGGAGT
AGAATACTGT TTCAAACTAC CTGGTGTATT TATTAATTTT GGAACTGTAT
GTGTGTGTCA TACATCTTCA TAGTTACGAG TTTAAGATGG ATGGAAATAT
CGATCTAGGA TAGGTATACA TGTTGATGTG GGTTTTACTG ATGCATATAC
ATGATGGCAT ATGCAGCATC TATTCATATG CTCTAACCTT GAGTACCTAT
CTATTATAAT AAACAAGTAT GTTTTATAAT TATTTTGATC TTGATATACT
TGGATGATGG CATATGCAGC AGCTATATGT GGATTTTTTT AGCCCTGCCT
TCATACGCTA TTTATTTGCT TGGTACTGTT TCTTTTGTCG ATGCTCACCC
TGTTGTTTGG TGTTACTTCT GC
SEQ ID NO: 47 Cas9
ATGGACTATAAGGACCACGACGGAGACTACAAGGATCATGATATTGATTACAAAG
ACGATGACGATAAGATGGCCCCAAAGAAGAAGCGGAAGGTCGGTATCCACGGAG
TCCCAGCAGCCGACAAGAAGTACAGCATCGGCCTGGACATCGGCACCAACTCTG
TGGGCTGGGCCGTGATCACCGACGAGTACAAGGTGCCCAGCAAGAAATTCAAGG
TGCTGGGCAACACCGACCGGCACAGCATCAAGAAGAACCTGATCGGAGCCCTGC
TGTTCGACAGCGGCGAAACAGCCGAGGCCACCCGGCTGAAGAGAACCGCCAGAA
GAAGATACACCAGACGGAAGAACCGGATCTGCTATCTGCAAGAGATCTTCAGCAA
CGAGATGGCCAAGGTGGACGACAGCTTCTTCCACAGACTGGAAGAGTCCTTCCT
GGTGGAAGAGGATAAGAAGCACGAGCGGCACCCCATCTTCGGCAACATCGTGGA
CGAGGTGGCCTACCACGAGAAGTACCCCACCATCTACCACCTGAGAAAGAAACTG
GTGGACAGCACCGACAAGGCCGACCTGCGGCTGATCTATCTGGCCCTGGCCCAC
ATGATCAAGTTCCGGGGCCACTTCCTGATCGAGGGCGACCTGAACCCCGACAAC
AGCGACGTGGACAAGCTGTTCATCCAGCTGGTGCAGACCTACAACCAGCTGTTCG
AGGAAAACCCCATCAACGCCAGCGGCGTGGACGCCAAGGCCATCCTGTCTGCCA
GACTGAGCAAGAGCAGACGGCTGGAAAATCTGATCGCCCAGCTGCCCGGCGAGA
AGAAGAATGGCCTGTTCGGAAACCTGATTGCCCTGAGCCTGGGCCTGACCCCCA
ACTTCAAGAGCAACTTCGACCTGGCCGAGGATGCCAAACTGCAGCTGAGCAAGG
ACACCTACGACGACGACCTGGACAACCTGCTGGCCCAGATCGGCGACCAGTACG
CCGACCTGTTTCTGGCCGCCAAGAACCTGTCCGACGCCATCCTGCTGAGCGACA
TCCTGAGAGTGAACACCGAGATCACCAAGGCCCCCCTGAGCGCCTCTATGATCAA
GAGATACGACGAGCACCACCAGGACCTGACCCTGCTGAAAGCTCTCGTGCGGCA
GCAGCTGCCTGAGAAGTACAAAGAGATTTTCTTCGACCAGAGCAAGAACGGCTAC
GCCGGCTACATTGACGGCGGAGCCAGCCAGGAAGAGTTCTACAAGTTCATCAAG
CCCATCCTGGAAAAGATGGACGGCACCGAGGAACTGCTCGTGAAGCTGAACAGA
GAGGACCTGCTGCGGAAGCAGCGGACCTTCGACAACGGCAGCATCCCCCACCAG
ATCCACCTGGGAGAGCTGCACGCCATTCTGCGGCGGCAGGAAGATTTTTACCCAT
TCCTGAAGGACAACCGGGAAAAGATCGAGAAGATCCTGACCTTCCGCATCCCCTA
CTACGTGGGCCCTCTGGCCAGGGGAAACAGCAGATTCGCCTGGATGACCAGAAA
GAGCGAGGAAACCATCACCCCCTGGAACTTCGAGGAAGTGGTGGACAAGGGCGC
TTCCGCCCAGAGCTTCATCGAGCGGATGACCAACTTCGATAAGAACCTGCCCAAC
GAGAAGGTGCTGCCCAAGCACAGCCTGCTGTACGAGTACTTCACCGTGTATAACG
AGCTGACCAAAGTGAAATACGTGACCGAGGGAATGAGAAAGCCCGCCTTCCTGA
GCGGCGAGCAGAAAAAGGCCATCGTGGACCTGCTGTTCAAGACCAACCGGAAAG
TGACCGTGAAGCAGCTGAAAGAGGACTACTTCAAGAAAATCGAGTGCTTCGACTC
CGTGGAAATCTCCGGCGTGGAAGATCGGTTCAACGCCTCCCTGGGCACATACCA
CGATCTGCTGAAAATTATCAAGGACAAGGACTTCCTGGACAATGAGGAAAACGAG
GACATTCTGGAAGATATCGTGCTGACCCTGACACTGTTTGAGGACAGAGAGATGA
TCGAGGAACGGCTGAAAACCTATGCCCACCTGTTCGACGACAAAGTGATGAAGCA
GCTGAAGCGGCGGAGATACACCGGCTGGGGCAGGCTGAGCCGGAAGCTGATCA
ACGGCATCCGGGACAAGCAGTCCGGCAAGACAATCCTGGATTTCCTGAAGTCCG
ACGGCTTCGCCAACAGAAACTTCATGCAGCTGATCCACGACGACAGCCTGACCTT
TAAAGAGGACATCCAGAAAGCCCAGGTGTCCGGCCAGGGCGATAGCCTGCACGA
GCACATTGCCAATCTGGCCGGCAGCCCCGCCATTAAGAAGGGCATCCTGCAGAC
AGTGAAGGTGGTGGACGAGCTCGTGAAAGTGATGGGCCGGCACAAGCCCGAGAA
CATCGTGATCGAAATGGCCAGAGAGAACCAGACCACCCAGAAGGGACAGAAGAA
CAGCCGCGAGAGAATGAAGCGGATCGAAGAGGGCATCAAAGAGCTGGGCAGCC
AGATCCTGAAAGAACACCCCGTGGAAAACACCCAGCTGCAGAACGAGAAGCTGTA
CCTGTACTACCTGCAGAATGGGCGGGATATGTACGTGGACCAGGAACTGGACAT
CAACCGGCTGTCCGACTACGATGTGGACCATATCGTGCCTCAGAGCTTTCTGAAG
GACGACTCCATCGACAACAAGGTGCTGACCAGAAGCGACAAGAACCGGGGCAAG
AGCGACAACGTGCCCTCCGAAGAGGTCGTGAAGAAGATGAAGAACTACTGGCGG
CAGCTGCTGAACGCCAAGCTGATTACCCAGAGAAAGTTCGACAATCTGACCAAGG
CCGAGAGAGGCGGCCTGAGCGAACTGGATAAGGCCGGCTTCATCAAGAGACAGC
TGGTGGAAACCCGGCAGATCACAAAGCACGTGGCACAGATCCTGGACTCCCGGA
TGAACACTAAGTACGACGAGAATGACAAGCTGATCCGGGAAGTGAAAGTGATCAC
CCTGAAGTCCAAGCTGGTGTCCGATTTCCGGAAGGATTTCCAGTTTTACAAAGTG
CGCGAGATCAACAACTACCACCACGCCCACGACGCCTACCTGAACGCCGTCGTG
GGAACCGCCCTGATCAAAAAGTACCCTAAGCTGGAAAGCGAGTTCGTGTACGGC
GACTACAAGGTGTACGACGTGCGGAAGATGATCGCCAAGAGCGAGCAGGAAATC
GGCAAGGCTACCGCCAAGTACTTCTTCTACAGCAACATCATGAACTTTTTCAAGAC
CGAGATTACCCTGGCCAACGGCGAGATCCGGAAGCGGCCTCTGATCGAGACAAA
CGGCGAAACCGGGGAGATCGTGTGGGATAAGGGCCGGGATTTTGCCACCGTGC
GGAAAGTGCTGAGCATGCCCCAAGTGAATATCGTGAAAAAGACCGAGGTGCAGA
CAGGCGGCTTCAGCAAAGAGTCTATCCTGCCCAAGAGGAACAGCGATAAGCTGAT
CGCCAGAAAGAAGGACTGGGACCCTAAGAAGTACGGCGGCTTCGACAGCCCCAC
CGTGGCCTATTCTGTGCTGGTGGTGGCCAAAGTGGAAAAGGGCAAGTCCAAGAA
ACTGAAGAGTGTGAAAGAGCTGCTGGGGATCACCATCATGGAAAGAAGCAGCTTC
GAGAAGAATCCCATCGACTTTCTGGAAGCCAAGGGCTACAAAGAAGTGAAAAAGG
ACCTGATCATCAAGCTGCCTAAGTACTCCCTGTTCGAGCTGGAAAACGGCCGGAA
GAGAATGCTGGCCTCTGCCGGCGAACTGCAGAAGGGAAACGAACTGGCCCTGCC
CTCCAAATATGTGAACTTCCTGTACCTGGCCAGCCACTATGAGAAGCTGAAGGGC
TCCCCCGAGGATAATGAGCAGAAACAGCTGTTTGTGGAACAGCACAAGCACTACC
TGGACGAGATCATCGAGCAGATCAGCGAGTTCTCCAAGAGAGTGATCCTGGCCG
ACGCTAATCTGGACAAAGTGCTGTCCGCCTACAACAAGCACCGGGATAAGCCCAT
CAGAGAGCAGGCCGAGAATATCATCCACCTGTTTACCCTGACCAATCTGGGAGCC
CCTGCCGCCTTCAAGTACTTTGACACCACCATCGACCGGAAGAGGTACACCAGCA
CCAAAGAGGTGCTGGACGCCACCCTGATCCACCAGAGCATCACCGGCCTGTACG
AGACACGGATCGACCTGTCTCAGCTGGGAGGCGACAAAAGGCCGGCGGCCACG
AAAAAGGCCGGCCAGGCAAAAAAGAAAAAGTAA
SEQ ID NO: 48: gRNA sequence
GTTTTAGAGCTAGAAATAGCAAGTTAAAATAAGGCTAGTCCGTTATCAACTTGAAA
AAGTGGCACCGAGTCGGTGC
SEQ ID NO: 49 miR528a nucleic acid sequence
GGGATTAGGGATAGGAATCAGGGTCAGTTTGCTCTGCTGGCTAGTAGCAGCAGC
GGTGGAAGGGGCATGCAGAGGAGGAGCACGAGCGAGGTGTGGCTGGAAGAAGC
AGCCGGGGCCTGTGTGCTATATACCCCTCGCAAGCTCTCCTCACTCCTCTCCTGT
GCCTGCCTCTTCCATTCCTTCTGCTACGCCATTATATGTTTGCAAGCGTCAGAAGT
GCATGCTTCTGCGCAGTTGCCCTGTGATGAGGCAACCAACAGCCAAACATCGCT
SEQ ID NO: 50: miR528b nucleic acid sequence
TCTTCTCCTCTCTAGTTCTGGGAGTTCCTGGCTGTAGCAGCAGCGGTGGAAGGGG
CATGCAGAGGAGCACGAACGAGGTGTGGTTGGCCCCTCGTTAGCTCTCCTGTGC
CTGCCTCTTCCATTCCTTCTGCTACGCTATGTCTGCAAGTAAAAAACTCAGAAGCG
CAACCGAGG
SEQ ID NO: 51: Lac3 target 1
ATCATCGATTCATCGTACACAGG
SEQ ID NO: 52: Lac3 protospacer 1
ATCATCGATTCATCGTACAC
SEQ ID NO: 53: Lac3 complete sgRNA 1:
ATCATCGATTCATCGTACACGTTTTAGAGCTAGAAATAGCAAGTTAAAATAAGGCT
AGTCCGTTATCAACTTGAAAAAGTGGCACCGAGTCGGTGC
SEQ ID NO: 54: Lac3 target2
CCGACGATGGGCGGCGGCGGCGG
SEQ ID NO: 55: Lac3 protospacer 2
ACGATGGGCGGCGGCGGCGG
SEQ ID NO: 56: Lac3 complete sgRNA 2:
ACGATGGGCGGCGGCGGCGGGTTTTAGAGCTAGAAATAGCAAGTTAAAATAAGG
CTAGTCCGTTATCAACTTGAAAAAGTGGCACCGAGTCGGTGC
SEQ ID NO: 57: Lac5 target1
GCGCGTCGTGGTCTCCGGCGAGG
SEQ ID NO: 58: Lac5 protospacer 1
GCGCGTCGTGGTCTCCGGCG
SEQ ID NO: 59: Lac5 complete sgRNA 1
GCGCGTCGTGGTCTCCGGCGGTTTTAGAGCTAGAAATAGCAAGTTAAAATAAGGC
TAGTCCGTTATCAACTTGAAAAAGTGGCACCGAGTCGGTGC
SEQ ID NO: 60: Lac5 target2
CCGCCTCGCCTTGCCGGAGCTCG
SEQ ID NO: 61: Lac5 protospacer 2
CCTCGCCTTGCCGGAGCTCG
SEQ ID NO: 62: Lac5 complete sgRNA 2
CCTCGCCTTGCCGGAGCTCGGTTTTAGAGCTAGAAATAGCAAGTTAAAATAAGGC
TAGTCCGTTATCAACTTGAAAAAGTGGCACCGAGTCGGTGC
SEQ ID NO: 63 DNA sequence of LbCpf1:
atgtctaagttggaaaaattcaccaactgttactctttgtctaagactttgagattcaaggccatcccagttggtaagaccca
agaaaacatcgacaacaagagactattagttgaagatgaaaagagagctgaagactacaagggtgtcaagaaattgt
tggacagatactacttgtcttttatcaacgacgttttgcattccatcaagctaaagaacttgaataactacatctctttgttcaga
aagaagactagaactgaaaaggaaaataaggaattggaaaacttggaaatcaacttgagaaaggaaattgctaagg
ctttcaagggtaatgaaggttacaagtctttattcaagaaagacatcattgaaaccattttgccagaatttttggatgataagg
atgaaattgctttggttaactctttcaacggtttcaccactgctttcactggtttcttcgacaacagagaaaacatgttctccgag
gaagctaaatccacttctattgctttcagatgtatcaacgaaaacttgacccgttacatctctaacatggacatttttgaaaag
gtcgacgccatctttgacaagcacgaagtccaagaaatcaaggaaaagatcttaaactccgactacgatgtcgaagatt
tcttcgaaggtgaattcttcaactttgttttaacccaagaaggtatcgatgtctacaacgccattatcggtggttttgtcactgaa
tctggtgaaaagatcaagggtttgaacgaatacattaacttgtacaaccaaaagaccaaacaaaaattgccaaagttca
agccattgtacaagcaagttttgtctgacagagaatctttgtctttttacggtgaagggtacacctctgacgaagaagtcttgg
aagtcttcagaaacactttgaacaagaactctgaaatcttctcctccatcaagaagttagaaaagttgttcaagaacttcga
tgaatactcttctgctggtatcttcgttaagaacggtccagccatctctaccatttctaaggatatctttggtgaatggaacgtc
attagagacaaatggaacgctgaatacgatgacatccatttgaagaaaaaggctgttgtcaccgaaaagtacgaagac
gacagaagaaaatccttcaagaagatcggttccttctccttggaacaattacaagaatacgccgatgccgatttgtccgtt
gtcgaaaaattgaaggaaattattattcaaaaggttgatgaaatttacaaagtttacggttcctctgaaaagttattcgatgct
gatttcgtcttggaaaagtctttgaagaagaacgacgctgttgtcgctatcatgaaggacttgttggactctgtcaaatctttcg
aaaactatatcaaggccttcttcggtgaaggtaaggaaactaacagagatgaatccttctacggtgactttgtcttggcttac
gatattttgttgaaggttgaccacatctacgatgccatcagaaactacgttactcaaaagccatactctaaggacaaattca
agttgtacttccaaaacccacaattcatgggtggttgggataaggacaaggaaactgactacagagctaccattttgagat
acggttccaagtactacttggccatcatggacaagaagtacgccaagtgtttgcaaaagattgacaaggacgatgtcaa
cggtaactacgaaaagattaactacaagttgttgccaggtccaaacaagatgttgccaaaggttttcttctccaaaaagtg
gatggcttactacaacccatctgaagacatccaaaagatctacaagaacggtactttcaaaaagggtgacatgttcaact
taaacgactgtcacaagttgatcgacttcttcaaggactccatctctagatacccaaaatggtccaacgcttacgatttcaa
cttctctgaaactgaaaaatacaaggatattgctggtttctaccgtgaagtcgaggaacaaggttataaggtttctttcgaat
ccgcttctaagaaagaagttgacaaattagtcgaagaaggtaagttgtacatgttccaaatctacaacaaagatttctccg
acaagtctcacggtactccaaacttgcacaccatgtacttcaagttgctattcgatgaaaacaaccacggtcaaatcagat
tgtctggtggtgctgaattgttcatgagacgtgcttctctaaagaaggaagaattagtcgtccacccagctaactctccaatt
gccaacaagaacccagacaaccctaagaagaccaccactttgtcctacgacgtttacaaggacaagagattctccga
agaccaatacgaattgcacattccaattgctatcaacaagtgtccaaagaacatcttcaagatcaacactgaagtcaga
gttttgttaaagcacgatgacaacccttacgttattggtatcgaccgtggtgaaagaaatttgttgtacattgttgttgttgacgg
taagggtaacatcgttgaacaatactccttgaacgaaatcatcaacaacttcaacggtattagaatcaagactgattacca
ctctttgttggataagaaggaaaaggaacgttttgaagctcgtcaaaactggacctctattgaaaacatcaaagaattgaa
ggctggttacatcagtcaagttgtccacaagatctgtgaattggtcgagaagtacgatgccgttattgccttggaagatttga
actctggttttaagaactctcgtgtcaaggttgaaaagcaagtctaccaaaagttcgaaaagatgttaatcgacaaattga
actacatggttgacaagaaatccaacccatgtgctaccggtggtgctttgaaaggttaccaaatcaccaacaaattcgaa
tctttcaaatctatgtccactcaaaacgggttcatcttctacattccagcttggttgacctccaagatcgacccatctaccggttt
cgttaacttgttgaagaccaagtacacttccattgctgattccaagaagttcatctcttctttcgacagaatcatgtacgttcca
gaagaagacttgttcgaattcgccttggactataagaacttctccagaaccgatgctgactacattaagaaatggaaattgt
actcctacggtaacagaatcagaattttcagaaacccaaagaaaaacaacgttttcgattgggaagaagtttgtttgacttc
tgcctacaaggaattattcaacaaatacggtatcaactaccaacaaggtgatatcagagctttgttgtgtgaacaatctgac
aaggctttctactcttccttcatggctttgatgtccttgatgttgcaaatgagaaactccatcactggtagaactgatgtcgactt
cctcatttctccagttaagaattctgacggtattttctacgactctagaaattacgaagctcaagaaaacgctattttgccaaa
gaacgctgatgctaacggtgcttacaatattgctagaaaggttttgtgggctatcggtcaattcaagaaggctgaagacga
aaagctagacaaggtcaagattgctatttctaacaaggaatggttggaatacgctcaaacctccgtcaagcac
SEQ ID NO: 64: protein sequence of LbCpf1:
MSKLEKFTNCYSLSKTLRFKAIPVGKTQENIDNKRLLVEDEKRAEDYKGVKKLLDRYYL
SFINDVLHSIKLKNLNNYISLFRKKTRTEKENKELENLEINLRKEIAKAFKGNEGYKSLFK
KDIIETILPEFLDDKDEIALVNSFNGFTTAFTGFFDNRENMFSEEAKSTSIAFRCINENLT
RYISNMDIFEKVDAIFDKHEVQEIKEKILNSDYDVEDFFEGEFFNFVLTQEGIDVYNAIIG
GFVTESGEKIKGLNEYINLYNQKTKQKLPKFKPLYKQVLSDRESLSFYGEGYTSDEEV
LEVFRNTLNKNSEIFSSIKKLEKLFKNFDEYSSAGIFVKNGPAISTISKDIFGEWNVIRDK
WNAEYDDIHLKKKAVVTEKYEDDRRKSFKKIGSFSLEQLQEYADADLSVVEKLKEIIIQK
VDEIYKVYGSSEKLFDADFVLEKSLKKNDAVVAIMKDLLDSVKSFENYIKAFFGEGKET
NRDESFYGDFVLAYDILLKVDHIYDAIRNYVTQKPYSKDKFKLYFQNPQFMGGWDKDK
ETDYRATILRYGSKYYLAIMDKKYAKCLQKIDKDDVNGNYEKINYKLLPGPNKMLPKVF
FSKKVVMAYYNPSEDIQKIYKNGTFKKGDMFNLNDCHKLIDFFKDSISRYPKWSNAYDF
NFSETEKYKDIAGFYREVEEQGYKVSFESASKKEVDKLVEEGKLYMFQIYNKDFSDKS
HGTPNLHTMYFKLLFDENNHGQIRLSGGAELFMRRASLKKEELVVHPANSPIANKNPD
NPKKTTTLSYDVYKDKRFSEDQYELHIPIAINKCPKNIFKINTEVRVLLKHDDNPYVIGID
RGERNLLYIVVVDGKGNIVEQYSLNEIINNFNGIRIKTDYHSLLDKKEKERFEARQNWT
SIENIKELKAGYISQVVHKICELVEKYDAVIALEDLNSGFKNSRVKVEKQVYQKFEKMLI
DKLNYMVDKKSNPCATGGALKGYQITNKFESFKSMSTQNGFIFYIPAWLTSKIDPSTG
FVNLLKTKYTSIADSKKFISSFDRIMYVPEEDLFEFALDYKNFSRTDADYIKKWKLYSYG
NRIRIFRNPKKNNVFDWEEVCLTSAYKELFNKYGINYQQGDIRALLCEQSDKAFYSSF
MALMSLMLQMRNSITGRTDVDFLISPVKNSDGIFYDSRNYEAQENAILPKNADANGAY
NIARKVLWAIGQFKKAEDEKLDKVKIAISNKEWLEYAQTSVKH
SEQ ID NO: 65: Replacement sequence of LAC3 (bold font is artificial miR528
binding site):
CACAGGATCAGAGAGCTTAATTAGTTCTAGCTCTGGACGATGCCACTGCGCCAAC
GTCCGACG
SEQ ID NO: 66: Replacement sequence of LAC3 (bold font is artificial miR528
binding site):
GCGAGGCCAAGCCGCCGCCGCCGCCTTUTCCGCTTGTCCGTTTCTCGCCCTCGC
CGTCGTCCTCCT
SEQ ID NO: 72: RNA sequence of SEQ ID NO: 35 (miR528a complete sgRNA 1)
ucagggucag uuugcucugc guuuuagagc uagaaauagc aaguuaaaau aaggcuaguc
cguuaucaac uugaaaaagu ggcaccgagu cggugc
SEQ ID NO: 73: RNA sequence of SEQ ID NO: 38 (mi528a complete sgRNA 2)
ugcgcaguug cccugugaug guuuuagagc uagaaauagc aaguuaaaau aaggcuaguc
cguuaucaac uugaaaaagu ggcaccgagu cggugc
SEQ ID NO: 74: RNA sequence of SEQ ID NO: 42 (mi528b complete sgRNA 1)
ucucuaguuc ugggaguucc guuuuagagc uagaaauagc aaguuaaaau aaggcuaguc
cguuaucaac uugaaaaagu ggcaccgagu cggugc
SEQ ID NO: 75: RNA sequence of SEQ ID NO: 45 (miR528b complete sgRNA 2)
aaaacucaga agcgcaaccg guuuuagagc uagaaauagc aaguuaaaau aaggcuaguc
cguuaucaac uugaaaaagu ggcaccgagu cggugc
SEQ ID NO: 76: RNA sequence of SEQ ID NO: 53 (Lac3 complete sgRNA 1)
aucaucgauu caucguacac guuuuagagc uagaaauagc aaguuaaaau aaggcuaguc
cguuaucaac uugaaaaagu ggcaccgagu cggugc
SEQ ID NO: 77: RNA sequence of SEQ ID NO: 56 (Lac3 complete sgRNA 2)
acgaugggcg gcggcggcgg guuuuagagc uagaaauagc aaguuaaaau aaggcuaguc
cguuaucaac uugaaaaagu ggcaccgagu cggugc
SEQ ID NO: 78: RNA sequence of SEQ ID NO: 59 (Lac5 complete sgRNA 1)
GCGCGUCGUGGUCUCCGGCGGUUUUAGAGCUAGAAAUAGCAAGUUAAAAUAAG
GCUAGUCCGUUAUCAACUUGAAAAAGUGGCACCGAGUCGGUGC
SEQ ID NO: 79: RNA sequence of SEQ ID NO: 62 (Lac5 complete sgRNA 2)
ccucgccuug ccggagcucg guuuuagagc uagaaauagc aaguuaaaau aaggcuaguc

| | |
|---|---|
| cguuaucaac uugaaaaagu ggcaccgagu cggugc | 96 |

1. A method of altering resistance to lodging in a plant, the method comprising altering the expression or levels of at least one laccase gene and/or altering the expression or activity of miR528.

2. The method of claim 1, wherein the method increases resistance to lodging in a plant, and wherein the method comprises increasing the expression of at least one laccase gene and/or decreasing the expression or activity of miR528.

3. (canceled)

4. The method of claim 1, wherein the method comprises introducing and expressing a nucleic acid construct comprising at least one nucleic acid sequence wherein the nucleic acid sequence encodes a laccase 3 polypeptide as defined in SEQ ID NO: 1 or a functional variant or homologue thereof and/or a laccase 5 polypeptide as defined in SEQ ID NO: 4 or a functional variant or homologue thereof operably linked to a regulatory sequence.

5. (canceled)

6. (canceled)

7. (canceled)

8. The method of claim 2, wherein the method comprises decreasing the activity of miR528 wherein the activity of miR528 is decreased using at least one miR528 inhibitor wherein the miR528 inhibitor is an RNA molecule comprising an RNA sequence as defined in SEQ ID NO: 8 or a functional variant thereof or wherein the activity of miR528 is decreased by introducing and expressing a nucleic acid construct comprising a nucleic acid sequence as defined in SEQ ID NO: 7 or 16 or a functional variant thereof operably linked to a regulatory sequence.

9. (canceled)

10. The method of claim 1, wherein the method comprises introducing at least one mutation into at least one laccase gene, wherein the laccase gene comprises at least one mutation in the miR528 binding site and/or introducing at least one mutation into a miR528 a and/or b gene or the miR528 promoter, wherein the laccase gene is selected from laccase 3 and laccase 5 and wherein the laccase 3 gene encodes a polypeptide as defined SEQ ID NO: 1 and wherein the laccase 5 gene encodes a polypeptide as defined in SEQ ID NO: 4.

11. (canceled)

12. (canceled)

13. (canceled)

14. (canceled)

15. The method of claim 1, wherein the method decreases resistance to lodging in a plant, and wherein the method comprises decreasing the expression of at least one laccase gene and/or increasing the expression or activity of miR528.

16. (canceled)

17. The method of claim 15, wherein the method comprises introducing at least one mutation into at least one laccase gene and/or promoter, wherein the mutation decreases the expression of the laccase nucleic acid compared to a wild-type or control polypeptide.

18. (canceled)

19. (canceled)

20. (canceled)

21. The method of claim 15, wherein the method comprises introducing and expressing a nucleic acid construct comprising at least one nucleic acid wherein the nucleic acid encodes a miR528 as defined in SEQ ID NO: 10 or a functional variant thereof operably linked to a regulatory sequence or wherein the method comprises introducing an miR528 comprising SEQ ID NO: 9 or a functional variant thereof.

22. (canceled)

23. (canceled)

24. (canceled)

25. (canceled)

26. The method of claim 1, wherein the plant is maize.

27. (canceled)

28. (canceled)

29. A genetically altered plant, part thereof or plant cell, wherein said plant is characterised by altered expression or levels of at least one laccase gene and/or altered expression or activity of miR528.

30. (canceled)

31. (canceled)

32. The genetically altered plant of claim 29, wherein the plant expresses a nucleic acid construct comprising at least one nucleic acid wherein the nucleic acid encodes a laccase 3 polypeptide as defined in SEQ ID NO: 1 or a functional variant or homologue thereof and/or a nucleic acid encoding a laccase 5 polypeptide as defined in SEQ ID NO: 4 or a functional variant or homologue thereof operably linked to a regulatory sequence.

33. (canceled)

34. (canceled)

35. (canceled)

36. The genetically altered plant of claim 29, wherein the plant expresses at least one miR528 inhibitor wherein the miR528 inhibitor is an RNA molecule comprising an RNA sequence as defined in SEQ ID NO: 8 or a functional variant thereof or wherein the miR528 inhibitor is a nucleic acid construct comprising a nucleic acid sequence as defined in SEQ ID NO: 7 or 16 or a functional variant thereof operably linked to a regulatory sequence.

37. (canceled)

38. The genetically altered plant of claim 29, wherein the plant comprises at least one mutation in at least one nucleic acid encoding a laccase nucleic acid, preferably wherein the laccase nucleic acid is selected from laccase 3 and 5, and wherein the mutation is in a miR528 binding site and/or at least one mutation in a miR528 a and/or b gene or the miR528 promoter.

39. (canceled)

40. (canceled)

41. (canceled)

42. (canceled)

43. (canceled)

44. The genetically altered plant of claim 38, wherein the plant expresses a nucleic acid construct comprising at least one nucleic acid wherein the nucleic acid encodes a miR528 as defined in SEQ ID NO: 10 or a functional variant thereof operably linked to a regulatory sequence or wherein the plant expresses a miR528 comprising SEQ ID NO: 9 or a functional variant thereof.

45. The genetically altered plant of claim 38, wherein the plant comprises at least one mutation in at least one nucleic acid encoding a laccase nucleic acid, preferably wherein the laccase nucleic acid is selected from laccase 3 and 5, and wherein the mutation decreases the expression of the laccase nucleic acid compared to a wild-type or control polypeptide.

46. (canceled)

47. (canceled)

48. (canceled)

49. The genetically altered plant of claim 29, wherein the plant is maize.

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. A nucleic acid construct comprising a nucleic acid sequence encoding a miR528 inhibitor as defined in SEQ ID NO: 7 or 16 or a functional variant thereof.

74. (canceled)

75. A miR528 inhibitor comprising an RNA molecule with an RNA sequence as defined in SEQ ID NO: 8 or a functional variant thereof.

76. (canceled)

77. (canceled)

78. (canceled)

79. (canceled)

80. (canceled)

81. (canceled)

82. (canceled)

83. (canceled)

84. A nucleic acid construct comprising a nucleic acid sequence encoding at least one DNA-binding domain that can bind to at least one miR528 gene or at least one DNA-binding domain that can bind to at least one laccase 3 gene or at least one DNA-binding domain that can bind to at least one laccase 5 gene wherein the nucleic acid sequence encodes at least one protospacer element, and wherein the sequence of the protospacer element is selected from SEQ ID NO: 34, 37, 41, 44 or 52, 55, 58 or 61 or a sequence that is at least 90% identical to SEQ ID NO: 34, 37, 41, 44, 52, 55, 58 or 61.

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. A single guide (sg) RNA molecule wherein said sgRNA comprises a crRNA sequence and a tracrRNA sequence, wherein the crRNA sequence can bind to at least one sequence selected from SEQ ID NO: 33, 36, 40, 43 or at least one sequence selected from SEQ ID NO: 51, 54, 57 or 60 or a variant thereof.

98. (canceled)

99. (canceled)

100. (canceled)

101. (canceled)

102. (canceled)

103. (canceled)

104. (canceled)

105. (canceled)

106. (canceled)

107. (canceled)

108. A method for identifying and/or selecting a plant that will have increased lodging resistance, preferably compared to a wild-type or control plant, the method comprising detecting in the plant or plant germplasm at least one polymorphism or mutation in a laccase 3 gene and/or promoter, laccase 5 gene and/pr promoter or miR528a and/or b gene and/or promoter wherein said polymorphism or mutation results in an increased expression of laccase 3 and/or 5 and/or reduced expression/activity of miR528 compared to a plant without said mutation; and selecting said plant or progeny thereof.

109. The genetically altered plant part of claim 29, wherein the plant part is a seed.
